(19) United States
(12) Patent Application Publication
(10) Pub. No.: US 2008/0096830 A1
(43) Pub. Date: Apr. 24, 2008

(54) Anti-Inflammatory Macrolide Conjugates

(76) Inventors: Mladen Mercep, Zagreb (HR); Milan Mesic, Zagreb (HR); Stribor Markovic, Zagreb (HR); Dijana Pesic, Sibenik (HR); Ivana Ozimec Landek, Zagreb (HR); Marijana Komac, Zagreb (HR); Oresta Makaruha Stegic, Zagreb (HR); Selvira Selmani, Zagreb (HR); Linda Tomaskovic, Zagreb (HR); Mihailo Banjanac, Zagreb (HR)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/813,882

(22) PCT Filed: Jan. 13, 2006
(86) PCT No.: PCT/IB06/01079

§ 371 Date: Jul. 13, 2007

Related U.S. Application Data
(60) Provisional application No. 60/643,931, filed on Jan. 13, 2005.

(51) Int. Cl. A61K 31/7052; A61K 31/395; A61P 29/00; C07D 495/04; C07H 17/08; C07D 498/04; A61P 37/00; A61K 31/424

(52) U.S. Cl. 514/29; 514/375; 514/410; 514/431; 536/7.3; 540/467

(57) ABSTRACT

The present invention relates (a) to new compounds represented by Formula I: 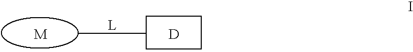 wherein M represents a macrolide subunit (macrolide moiety) derived from macrolide possessing the property of accumulation in inflammatory cells, D represents a dibenzo[e,h]azulene subunit with anti-inflammatory, analgesic and/or antipyretic activity and L represents a linking group covalently linking M and D; (b) to their pharmacologically acceptable salts, prodrugs and solvates, (c) to processes and intermediates for their preparation, and (d) to their use in the treatment of inflammatory diseases and conditions in humans and animals.

This application claims priority to U.S. Provisional Application No. 60/643,931 filed Jan. 13, 2005, herein incorporated by reference in its entirety.

### FIELD OF THE INVENTION

The present invention relates to new anti-inflammatory compounds represented by the general structure I, to their pharmaceutically acceptable salts and solvates, to processes and intermediates for their preparation and to the use of these compounds in the treatment of inflammatory diseases and conditions in humans and animals.

### TECHNICAL PROBLEM

The invention is directed to solving the technical problem of providing novel targeted anti-inflammatory agents. More specifically, the invention provides anti-inflammatory agents wherein the anti-inflammatory action of a dibenzoazulene moeity. The compounds of the invention are responsive to this problem by virtue of their anti-inflammatory activity and their ability to accumulate in various immune cells recruited to the locus of inflammation.

### BACKGROUND OF THE INVENTION

Anti-inflammatory medicaments having different mechanisms of action act on particular inflammation mediators, thus providing a therapeutic effect. Due to differences not only in mechanisms of action but also in the particular inflammation mediators inhibited, steroid and nonsteroid medicaments possess different profiles of anti-inflammation effects, hence certain medicaments may be more suitable than others for particular conditions. Moreover, most nonsteroid anti-inflammatory medicaments are not absolutely specific and their use is accompanied by unfavorable side-effects especially when used in greater dosages or over long periods of time. It is known that many nonsteroid anti-inflammatory medicaments act as inhibitors of endogenous COX-1 enzyme, which is very important in maintaining the integrity of the gastric mucosa. Thus, the use of these medicaments often causes injuries of the gastric mucosa and even bleeding. (Warner T. D. Proc. Natl. Acad. Sci. U.S.A. 1999, 96, 7563-7568.) Therefore, agents that selectively inhibit COX-2 but not COX-1 are in principle preferable for treatment of inflammatory diseases. Additionally, some anti-inflammatory compounds (such as theophylline) are known to have a very narrow therapeutic index (one in which small increases in dosage cause toxic effect and/or small decreases in dosage ablate therapeutic effect), which limits their usage.

Recently, the nonsteroidal antiinflammatory drug celecoxib that specifically blocks COX-2 has been approved by the FDA for use in the treatment of rheumatoid arthritis (Luong et al. Ann. Pharmacother. 2000, 34, 743-760). COX-2 is also expressed in many cancers and precancerous lesions, and there is accumulating evidence that selective COX-2 inhibitors may be useful for treating and preventing colorectal and other cancers (Taketo, M. M., J. Natl. Cancer Inst. 1998, 90, 1609-1620, Fournier et. al. J. Cell Biochem. Suppl. 2000, 34, 97-102).

It is known from the art (WO2003084962A1, WO2003084961A1, WO2003084964A1, WO2003099827A1, WO2003099822A2, WO2003097648A1, WO2003097649, WO2003099823, WO2004/078763A1) that dibenzoazulenes show anti-inflammatory activity, notably inhibiton of cytokines like TNF-α; some dibenzoazulenes are known to have potential use in different CNS disorders (WO2005/041856A1, WO2005/049011A1, WO2005/049010A1, WO2005/-049015A1, WO2005/049036A1, WO2005/049020A1, WO2005/049016A1). Each of these publications are incorporated herein by reference in their entirety.

Macrolides such as macrolide antibiotics accumulate preferentially within different cells of subjects administered such molecules, especially within phagocyte cells such as mononuclear peripheral blood cells, peritoneal and alveolar macrophages as well as in the liquid surrounding the bronchoalveolar epithelium (Glaude R. P. et al. Antimicrob. Agents Chemother., 1989, 33, 277-282; Olsen K. M. et al. Antimicrob. Agents Chemother. 1996, 40, 2582-2585). Moreover, relatively weak anti-inflammatory effects of some macrolides have been described. For example, the anti-inflammatory effect of erythromycin derivatives (Labro M. T. J. Antimicrob. Chemother., 1998, 41, 37-46; WO 00/42055) and azithromycin derivatives has recently been described (EP 0283055). Anti-inflammatory effects of some macrolides are also known from in vitro and in vivo studies in experimental animal models such as zimosane induced peritonitis in mice (Mikasa et al. J Antimicrob. Chemother. 1992, 30, 339-348) and endotoxin-induced neutrophil accumulation in rat trachea (J. Immunol. 1997, 159, 3395-4005). The modulating effect of macrolides upon cytokines such as interleukin 8 (IL-8) (Am. J. Respir. Crit. Care Med. 1997, 156, 266-271) or interleukin 5 (IL-5) (EP 0775489 and EP 0771564) is known as well.

In 1975, TNF-α was defined as an endotoxin-induced serum factor causing tumor necrosis in vitro and in vivo (Carswell E. A. et al. Proc. Natl. Acad. Sci. U.S.A. 1975, 72, 3666-3670). In addition to antitumor activity, TNF-α has several other biologic activities, which are important in homeostasis as well as in pathophysiological conditions. The main sources of TNF-α are monocytes, macrophages, T-lymphocytes and mast cells.

The finding that anti-TNF-α antibodies (cA2) are effective in the treatment of patients suffering from rheumatoid arthritis (RA) (Elliot M. et al. Lancet 1994, 344, 1105-1110) intensified the interest to find new TNF-α inhibitors as possible potent medicaments for RA. Rheumatoid arthritis is an autoimmune chronic inflammatory disease characterized by irreversible pathological changes of the joints. In addition to RA, TNF-α antagonists are also applicable to several other pathological conditions and diseases such as spondylitis, osteoarthritis, gout and other arthritic conditions, sepsis, septic shock, toxic shock syndrome, atopic dermatitis, contact dermatitis, psoriasis, glomerulonephritis, lupus erhythematosus, scleroderma, asthma, cachexia, chronic obstructive lung disease, congestive heart failure, insulin resistance, lung fibrosis, multiple sclerosis, Crohn s disease, ulcerative colitis, viral infections and AIDS.

Proof of biological importance of TNF-α was obtained in in vivo experiments in mice having inactivated genes for TNF-α or its receptor. Such animals were resistant to collagen-induced arthritis (Mori L. et al. J. Immunol. 1996, 157, 3178-3182) and to endotoxin-induced shock (Pfeffer K. et al. Cell 1993, 73, 457-467). In experiments with animals having an increased TNF-α level, a chronic inflammatory polyarthritis appeared (Georgopoulos S. et al. J. Inflamm. 1996, 46, 86-97; Keffer J. et al. EMBO J. 1991, 10, 4025-4031), which was palliated by inhibitors of TNF-α production. The treatment of such inflammatory and pathologic conditions usually includes the application of nonsteroid anti-inflammatory medicaments, in severe cases, however, gold salts, D-pencillinamine or methotrexate are administered. The mentioned medicaments act symptomatically and do not stop the pathological process. New approaches in therapy of rheumatoid arthritis have been established using medicaments such as tenidap, leflunomide, cyclosporin, FK-506 and biomolecules neutralizing the activity of TNF-α. At present, the soluble TNF receptor named etanercept (Enbrel, Immunex/Wyeth) and mouse and human chimeric monoclonal antibody named infliximab (Remicade, Centocor) are available on the market. In addition to RA-therapy, etanercept and infliximab are also approved for the treatment of Crohn s disease (Exp. Opin. Invest. Drugs 2000, 9, 103).

International Publication No. WO 02/055531 A1, herein incorporated by reference in its entirety, discloses conjugate compounds represented by the Formula Ia: 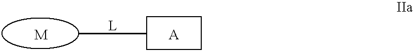
wherein M represents a macrolide subunit possessing the property of accumulation in inflammatory cells, A represents an anti-inflammatory subunit that can be steroid or nonsteroidal, and L represents a linker molecule linking M and A, (b) their pharmacologically acceptable salts, prodrugs and solvates, (c) processes and intermediates for their preparation, and (d) their use in the treatment of inflammatory diseases and conditions in humans and animals. In WO 02/05531, a number of the conjugate steroid-macrolide compounds are linked with the steroid subunit at the N/9a-position of macrolide ring.

U.S. Published Application 2004 0014685 and International Publication No. WO 04/005310 A2, herein incorporated by reference in their entirety, relate to compounds represented by Formula IIIa. 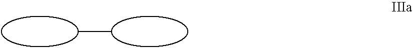
wherein M represents a macrolide subunit (macrolide moiety) derived from macrolide possessing the property of accumulation in inflammatory cells, S represents a steroid subunit derived from a steroid drug with anti-inflammatory activity and L represents a linker molecule linking M and S to their pharmaceutically acceptable salts and solvates processes and intermediates for their preparation and to their use in the treatment of inflammatory diseases and conditions in humans and animals.

US Published Application 20040077612 herein incorporated by reference in its entirety relates to new compounds represented by Formula IVa. 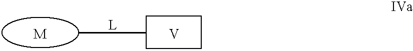
wherein M represents a macrolide subunit (macrolide moiety) derived from macrolide possessing the property of accumulation in inflammatory cells, V represents an anti-inflammatory steroid or non steroid subunit or an anti neoplastic or antiviral subunit and L represents a linking group covalently linking M and V to their pharmaceutically acceptable salts and solvates processes and intermediates for their preparation and to their use in the treatment of inflammatory diseases and conditions in humans and animals.

US Published Application 2004 0097434 and International Publication No. WO 04/005309, each of which are herein incorporated by reference in their entirety relates to new compounds represented by formula Va. 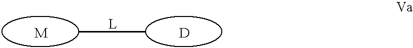
wherein M represents a macrolide subunit (macrolide moiety) derived from macrolide possessing the property of accumulation in inflammatory cells, D represents a nonsteroidal subunit (nonsteroidal moiety) derived from a nonsteroidal drug with anti-inflammatory, analgesic and/or antipyretic activity (NSAID) and L represents a linking group covalent linking M and D to their pharmaceutically acceptable salts and solvates processes and intermediates for their preparation and to their use in the treatment of inflammatory diseases and conditions in humans and animals.

US Published Application 20050080003, herein incorporated by reference in its entirety, describes yet further conjugate compounds having a steroid or non-steroidal anti-inflammatory subunit D linked via the chain L to position N/9a of an aglycone type macrolide subunit.

US Published Application 20040087517 and International Publication WO2003/070174 disclose a conjugate of (i) a “transportophore” and (ii) a “non-antibiotic therapeutic agent” covalently linked by a bond or a linker incorporating the transportophore. The transportophore and conjugate must have an immune selectivity ratio of at least 2. “Transportophore” is broadly defined as a compound, a portion of which resembles and is recognized as a substrate for transport protein(s).

### SUMMARY OF THE INVENTION

New compounds represented by the Formula I, representing the subject of the present invention, their pharmacologically acceptable salts, hydrates, prodrugs and pharmaceutical compositions comprising them have hitherto not been described. The invention is directed to solving the technical problem of providing novel targeted anti-inflammatory agents. The compounds of the invention are responsive to this problem by virtue of their anti-inflammatory activity and their ability to accumulate in various immune cells recruited to the locus of inflammation. Moreover, no compound representing the subject of the present invention has been described either as an anti-inflammatory substance or as an inhibitor of TNF-α or inhibitor of COX-1/COX-2 or inhibitor of 5-LOX or an inhibitor of IL-1β.

Compounds of the Formula I differ from hitherto known compounds in that they combine the anti-inflammatory properties of the dibenzo[e,h]azulene moiety with the accumulation properties afforded by the macrolide moiety, which, when conjoined, are recruited (along with the immune system cells in which macrolides preferentially accumulate) to the organs or tissues afflicted in inflammatory states, and result in substantially more localized and/or intensified abatement of the inflammation. Such action of the new compounds represented by the structure I arises from the macrolide portion M due to the specific pharmacokinetic properties of macrolides to accumulate within immune cells of inflammatory profile, such as phagocytes, including polymorphonuclear cells, eosinophils, peripheral and alveolar phagocytes, etc. Compounds of the Formula I possess improved pharmacokinetic and/or safety profiles, and present fewer and/or more benign side-effects.

The compounds represented by the Formula I, which are the subject of the present invention, isomeric forms of such compounds, their pharmacologically acceptable salts, prodrugs, solvates and pharmaceutical compositions comprising them are not believed to have been previously described. Moreover, none of the compounds of the present invention has been described either as an anti-inflammatory substance or as an inhibitor of eosinophilic accumulation in organs or tissues.

The present invention is directed to

(a) new “hybrid” or conjugate compounds represented by the Formula I 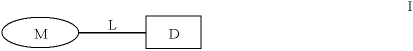
wherein M represents a macrolide subunit possessing the property of accumulation in inflammatory cells, D represents a dibenzo[e,h]azulene subunit with anti-inflammatory, analgesic and/or antipyretic activity, and L represents a linking group covalently linking M and D;

(b) compositions containing one or more of the foregoing compounds in an amount effective to combat inflammation and thereby treat disorders and conditions involving inflammation in mammals, including humans; and

(c) methods for using these compounds to treat such disorders and conditions.

The present compounds advantageously provide an improved therapeutic effect and/or an improved side effect profile.

### DETAILED DESCRIPTION OF THE INVENTION

Suitable macrolide subunits for the hybrid compounds of the present invention can be selected without limitation from multi-member lactonic ring molecules, wherein “member” refers to the carbon atoms or heteroatoms in the ring, and “multi” is a number greater than about 10, preferably from 10 to about 50, more preferably 12-, 14-, 15-, 16-, 17- and 18-member lactonic ring macrolides. 14- and 15-member ring macrolide subunits are particularly preferred, with azithromycin and its derivatives and erythromycin and its derivatives being more preferred, and with 9a-aza-9a-homoerythromycin and its derivatives being most preferred.

More specific nonlimiting examples of molecules from which the macrolide subunit can be selected are the following:

(i) Macrolide antibiotics, including azalides, for example erythromycin, dirithromycin, azithromycin, 9-dihydro-9-deoxo-9a-aza-9a-homoerythromycin, HMR 3004, HMR 3647, HMR 3787, josamycin, erythromycylamine, ABT 773 flurithromycin, clarithromycin, tylosin, tilmicosin, oleandomycin, desmycosin, CP-163505, roxithromycin, miocamycin and rokitamycin, and derivatives thereof, such as ketolides (e.g., 3-ketone), lactams (e.g., 8a- or 9a-lactams) and derivatives lacking one or more sugar moieties.

(ii) Macrolide immunosuppressants, such as FK 506, cyclosporin, amphotericin and rapamycin;

(iii) Macrolide antifungals with host cell inhibitory properties, such as bafilomycins, concanamycin, nystatin, natamycin, candicidin, filipin, etruscomycin, trichomycin.

Methodologies for the synthesis of the above macrolides not commercially available and synthetic manipulation of macrolides in general are known to those of ordinary skill in the art, or may be found in, for example: Denis A. et al. Bioorg. & Med. Chem. Lett 1999, 9, 3075-3080; Agouridas C. et al. J. Med. Chem. 1998, 41, 4080-4100; and EP-00680967 (1998); Sun Or Y. et al. J. Med. Chem. 2000, 43, 1045-1049; U.S. Pat. No. 0,574,7467 (1998); McFarland J. W. et al. J. Med. Chem. 1997, 40, 1041-1045; Denis A. at al. Bioorg. & Med. Chem. Lett. 1998, 8, 2427-2432; WO-09951616 (1999); Lartey et al. J Med. Chem. 1995, 38, 1793-1798; EP 0984019; WO 98/56801, each of which are herein incorporated by reference in their entirety.

Additional suitable macrolides are known, some being disclosed in Bryskier, A. J. et al. Macrolides, Chemistry, Pharmacology and Clinical Use; Arnette Blackwell: Paris, 1993, pp 485-491, 14(R)-hydroxyclarithromycin, erythromycin-11,12-carbonate, tri-O-acetyloleandomycin, spiramycin, leucomycin, midecamycin, rasaramycin incorporated by reference in its entirety; in Ma, Z. et al. Current Medicinal Chemisty-Anti-Infective Agents, 2002, 1, 15-34; also incorporated by reference in its entirety pikromycin, narbomycin, HMR-3562, CP-654743, CP-605006, TE-802, TE-935, TE-943, TE-806, 6,11-bridged ketolides, CP-544372, FMA-199, A-179461; and in Romo, D. et al. J. Am. Chem. Soc. 1998, 120; 12237-12254; also incorporated by reference in its entirety. See, in particular the structures and derivatives for 14- and 16-member ring macrolides at pp 487-491 of Bryskier, et al., and the various ketolide derivatives and syntheses in Ma et al., notably in all the structure tables and all the reaction schemes. All these macrolides after being conjugated to dibenzo[e,h]azulene subunits using a linker moiety are within the scope of the present invention. The foregoing specifically named or referenced macrolide compounds are commercially available or methods for their syntheses are known.

It is important that the macrolide subunit derive from a macrolide having the property of accumulating within immune system cells recruited to the site of inflammation, especially phagocytic cells. Most of the lactonic compounds defined above are known to have this property. For example, 14-membered macrolides such as erythromycin and its derivatives; 15-membered macrolides such as azithromycin and its derivatives, as well as 8a- and 9a-lactams and their derivatives; 16-membered macrolides such as tilmicosin, desmycosin; and spiramycin are known or expected to accumulate within immune system cells.

Additional examples of macrolides accumulating within specific classes of cells may be found in: Pascual A. et al. Clin. Microbiol. Infect. 2001, 7, 65-69. (Uptake and intracellular activity of ketolide HMR 3647 in human phagocytic and non-phagocytic cells); Hand W. L. et al. Int. J. Antimicrob. Agents, 2001, 18, 419-425. (Characteristics and mechanisms of azithromycin accumulation and efflux in human polymorphonuclear leukocytes); Amsden G. W. Int. J. Antimicrob. Agents, 2001, 18, 11-15. (Advanced-generation macrolides: tissue-directed antibiotics); Johnson J. D. et al. J. Lab. Clin. Med. 1980, 95, 429-439.(Antibiotic uptake by alveolar macrophages); Wildfeuer A. et al. Antimicrob. Agents Chemother. 1996, 40, 75-79. (Uptake of azithromycin by various cells and its intracellular activity under in vivo conditions); Scomeaux B. et al. Poult. Sci. 1998, 77, 1510-1521. (Intracellular accumulation, subcellular distribution, and efflux of tilmicosin in chicken phagocytes); Mtairag E. M. et al. J. Antimicrob. Chemother. 1994, 33, 523-536. (Investigation of dirithromycin and erythromycylamine uptake by human neutrophils in vitro); Anderson R. et al. J. Antimicrob. Chemother. 1988, 22, 923-933. (An in-vitro evaluation of the cellular uptake and intraphagocytic bioactivity of clarithromycin (A-56268, TE-031), a new macrolide antimicrobial agent); Tasaka Y. et al. Jpn. J. Antibiot. 1988, 41, 836-840. (Rokitamycin uptake by alveolar macrophages); Harf R. et al. J. Antimicrob. Chemother. 1988, 22, 135-140. (Spiramycin uptake by alveolar macrophages), herein incorporated by reference in their entirety.

Moreover, the presence of accumulating property within immune system cells recruited to the site of inflammation, especially phagocytic cells can be easily ascertained by a person of ordinary skill in the field of the invention, using one of the well-known assays for this purpose. For example, the procedure detailed by Olsen, K. M. et al. Anitmicrob. Agents & Chemother. 1996, 40, 2582-2585 can be used. Briefly, the cells to be tested, e.g., polymorphonuclear leukocytes can be obtained from venous blood of healthy volunteers by Ficoll-Hypaque centrifugation followed by 2% dextran sedimentation. Erythrocytes are removed by osmotic lysis, and PMN are evaluated by Trypan blue exclusion. Alternatively, other cell fractions can be separated and similarly tested. Tritiated macrolide compounds (e.g., 10 μM) are incubated with 2.5×10⁶ cells for 120 minutes (37° C., 5% CO₂, 90% relative humidity) and the cells are subsequently removed from compound-containing supernatant by centrifugation e.g., through a silicon oil-paraffin layer (86 vol %:14 vol %). The amount of compound is determined, e.g., by scintillation counting, and a score significantly elevated above background indicates accumulation of the macrolide in the cells being tested. See Bryskier et al. Macrolides, Chemistry, Pharmacology and Clinical Use; Arnette Blackwell: Paris, 1993 pp 375-386, at page 381, column 2, line 3. Alternatively, the compound is not radiolabeled but the amount of compound can be determined by HPLC.

Other assay methods that can be used are disclosed in Bryskier, A. J. et al. Macrolides, Chemistry, Pharmacology and Clinical Use; Arnette Blackwell: Paris, 1993 pp 375-386, incorporated by reference. See, in particular phagocytic uptake determination at pp 380-381 and the particular descriptions as to uptake and localization of macrolides at pp 381, 383 and 385 and the tables at 382 and 383.

In some preferred embodiments, this invention relates to compounds, their salts and solvates represented by the Formula I, wherein M specifically represents a 14- or 15-member lactonic ring macrolide subunit most preferably represented by the Formula II: 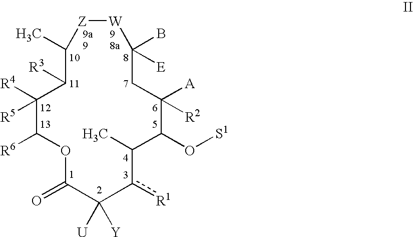
wherein
(i) Z and W independently are 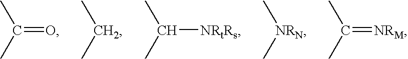
or a bond, wherein
Rₜ and Rₛ independently are H or alkyl (preferably methyl or H)
R_{M} is OH, OR^{p}, alkoxy or substituted alkoxy (in either Syn or Anti configurations or mixtures thereof)
R_{N} is H, R^{p}, alkyl, alkenyl, alkynyl, alkoxy, alkoxyalkyl or —C(═X)—NRₜRₛ;
X is O or S;

provided that Z and W cannot both simultaneously be 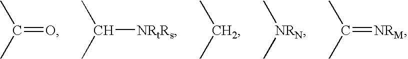
or a bond,
(ii) U and Y are independently H, halogen, alkyl, or hydroxyalkyl (preferably H, methyl or hydroxymethyl);
(iii) R¹ is hydroxy OR^{p}, —O—S², or ═O;
(iv) S¹ is H or a sugar moiety at position C/5 of the aglycone ring (e.g., a desozamine group) of the formula: 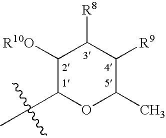
wherein
R⁸ and R⁹ are both hydrogen or together form a bond or R⁹ is hydrogen and R⁸ is —N(CH₃)R^{y}, wherein
R^{y} may be R^{p}, R^{z} or —C(O)R^{z}, wherein R^{z} is hydrogen or cycloalkyl (preferably cyclohexyl) or alkyl (preferably a C₁-C₇ alkyl) or alkenyl (preferably C₂-C₇-alkenyl) or alkynyl (preferably C₂-C₇-alkynyl) aryl or heteroaryl or can be alkyl substituted with C₁-C₇ alkyl or C₂-C₇ alkenyl or C₂-C₇ alknyl or aryl or heteroaryl. (R^{y} is preferably hydrogen, methyl, or ethyl);
R¹⁰ is hydrogen or R^{p};
   (v) S² is H or a sugar moiety at position C/3 of the aglycone ring (e.g., a cladinosyl group) of the formula 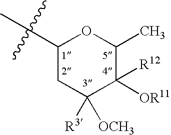
   wherein R^{3′} can be H or methyl and R¹¹ and R¹² are independently hydrogen, R¹¹ may be an R^{p} or R¹¹ and R¹² together form a bond;
   (vi) R² is H, hydroxy, OR^{p} group, alkoxy (preferably C₁-C₄ alkoxy, most preferably methoxy), substituted alkoxy;
   (vii) A is H or methyl;
   (viii) B is methyl or epoxy;
   (ix) E is H or halogen (preferably fluorine);
   (x) R³ is hydroxy, OR^{p} group or alkoxy (preferably C₁-C₄ alkoxy, most preferably methoxy), substituted alkoxy or R³ is a group that can combine with R⁵ to form a “bridge” (e.g., a cyclic carbonate or carbamate) or if W or Z is 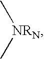
   R³ is a group that can combine with W or Z to form a “bridge” (e.g., a cyclic carbamate);
   (xi) R⁴ is C₁-C₄ alkyl (preferably methyl);
   (xii) R⁵ is H, hydroxy, OR^{p} group, C₁-C₄ alkoxy, substituted alkoxy or a group that may combine with R³ to form a bridge (e.g., a cyclic carbonate or carbamate);
   (xiii) R⁶ is H or C₁-C₄ alkyl (preferably methyl or ethyl); and
   R^{p} is a hydroxyl or amino protective group;
   wherein the subunit M has a linkage site through which it is linked to the subunit D via the linking group L. The linkage site is preferably at one or more of the following:
a. any reactive hydroxy, N, or epoxy group located on S¹, S², or an aglycone oxygen if S² is (or if both S² and S¹ are) cleaved off;
b. a reactive >N—R_{N}, —NRₜR^{s} or ═O group located on Z or W;
c. a reactive hydroxy group located at any one of R¹, R², R³, and R⁵;
d. any other group that can be first derivatized to a hydroxy or —NRₜRₛ, group and then linked to all or part of L (e.g., 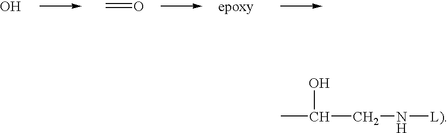
   One or more R^{p} groups may be independently present in the macrolide subunit of Formula II, wherein R^{p} represents a protective group which may be selected from alkyl (preferably methyl), alkanoyl (preferably acetyl), alkoxycarbonyl (preferably methoxycarbonyl or tert-butoxycarbonyl), arylmethoxycarbonyl (preferably benzyloxycarbonyl), aroyl (preferably benzoyl), arylalkyl (preferably benzyl), alkylsilyl (preferably trimethylsilyl) or alkylsilylalkoxyalkyl (preferably trimethylsilylethoxymethyl) group. The amino protecting groups may be removed by conventional techniques. Thus, for example acyl groups like alkanoyl, alkoxycarbonyl or aroyl may be removed by solvolysis, e.g. by hydrolysis under acidic or basic conditions. An arylmethoxycarbonyl group (benzyloxycarbonyl) may be cleaved by hydrogenolysis in the presence of a catalyst such as palladium-on-charcoal.
   L is a spacing or linking group that can be selected from a variety of linking groups providing the required spacing between M and D. For example and without limitation, L can be selected to be a linking group represented by the Formula IV:
   X¹—(CH₂)ₘ-Q-(CH₂)ₙ—X² IV

wherein

X¹ is selected from: —CH₂—, —OC(═O)—, —C(—O), NO—, —OC(═O)NH— or —C(═O)NH—;

X² is selected from: —NH—, —CH₂—, —NHC(—O)—, —OC(═O—O)—, —C(═O) or —O Q is —NH— or —CH₂— or absent;

wherein each —CH₂— or —NH— group may be optionally substituted by C₁-C₇-alkyl, C₂-C₇-alkenyl, C₂-C₇-alkynyl, C(O)R^{x}, C(O)OR^{x}, C(O)NHR^{x} wherein R^{x} may be C₁-C₇-alkyl, aryl or heteroaryl; the symbols m and n independently are a whole number from 0 to 4;

with the proviso that if Q=NH n cannot be zero.

Other linking groups can be used as long as they provide the necessary spacer, and can serve to link one subunit of the Formula I with the other, as is well-known in the art. Because the linking groups have only a linking role, their identity is not considered essential to the invention in its broadest embodiment.

In Formula I, D specifically represents a dibenzo[e,h]azulene subunit represented by the Formula III: 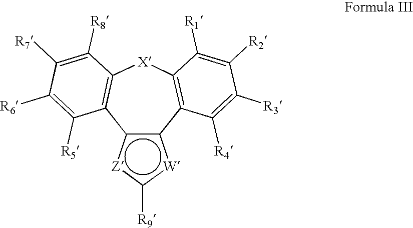
wherein,
X′ individually denotes —CH₂— or a heteroatom selected from the group consisting of —O—; —S—; or NR_{10′};
W′ and Z′ are independently —CH—, —S—, —O— or —NR_{11′}— with the proviso that W′ and Z′ can not simultaneously be —CH—, oxygen, or sulfur;
R₁′, R₂′, R₃′, R₄′, R₅′, R₆′, R₇′ and R₈′ independently from each other denote hydrogen or one or more identical or different substituents linked to one or more available carbon atoms, and may be halogen, C₁-C₄ alkyl, halo-C₁-C₄ alkyl, hydroxy, C₁-C₄ alkyoxy, C₁-C₄ alkanoyl, methansulfoanilide, amino, amino-C₁-C₄ alkyl, N—(C₁-C₄-alkyl)amino, N,N-di(C₁-C₄alkyl)amino, thiol, C₁-C₄ alkylthio, hydroxycarbonyl, formyl, cyano, C₁-C₄ alkyloxycarbonyl, C₁-C₇ alkylsulfonyl, C₁-C₇ alkylsulfinyl; hydroxy-C₁-C₇ alkylsulfonyl, hydroxy-C₁-C₇ alkylsulfinyl; amino-C₁-C₇ alkylsulfonyl, amino-C₁-C₇ alkylsulfinyl;

R₉′ is hydrogen, halo, an optionally substituted C₁-C₇ alkyl or C₂-C₇ alkenyl, C₂-C₇ alkynyl group, an optionally substituted aryl, heteroaryl or heterocyclic group, hydroxy, hydroxyalkyl, formyl, hydroxy-C₂-C₇ alkenyl, hydroxy-C₂-C₇ alkynyl, C₁-C₇ alkoxy, C₁-C₇ alkyloxoalkyl, thiol, thio-C₂-C₇ alkenyl, thio-C₂-C₇ alkynyl, C₂-C₇ alkylthiol, amino, N—(C₂-C₇-alkyl)amino, N,N-di(C₁-C₇-alkyl)amino, C₁-C₇ alkylamino, amino-C₂-C₇ alkenyl, amino-C₂-C₇ alkynyl, amino-C₁-C₇ alkoxy, C₁-C₇ alkanoyl, aroyl, oxo-C₁-C₇ alkyl, C₁-C₇ alkanoyloxy, carboxy, an optionally substituted C₁-C₇ alkyloxycarbonyl or aryloxycarbonyl, carbamoyl, N—(C₁-C₇-alkyl)carbamoyl, N,N-di(C₁-C₇-alkyl)carbamoyl, hydroxycarbonylalkyl, cyano, cyano-C₁-C₇ alkyl, sulfonyl, C₁-C₇ alkylsulfonyl, sulfinyl, C₁-C₇ alkylsulfinyl, hydroxy-C₁-C₇ alkylsulfonyl, hydroxy-C₁-C₇ alkylsulfinyl; amino-C₁-C₇ alkylsulfonyl, amino-C₁-C₇ alkylsulfinyl or nitro group or a group represented with the formula IIb:
Q₁-(CH₂)ₙ-Q₂-A′ IIb
wherein
Q₁ and Q₂ independently from each other have the meaning of oxygen, sulfur or of the following four groups: 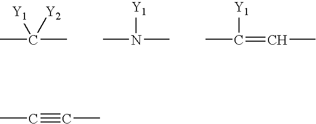
wherein the substituents
y₁ and y₂ independently from each other have the meaning of hydrogen, halogen, an optionally substituted C₁-C₄-alkyl or aryl hydroxy, C₁-C₄-alkoxy, C₁-C₄-alkanoyl, thiol, C₁-C₄-alkylthio, sulfonyl, C₁-C₄-alkylsulfonyl, sulfinyl, C₁-C₄-alkylsulfinyl, cyano, nitro, or together form a carbonyl or imino group, and A individually denotes an amino, N—(C₁-C₇-alkyl)amino, N,N-di(C₁-C₇-alkyl)amino, optionally substituted aryl, heterocyclic or heteroaryl selected from the group consisting of morpholine-4-yl, piperidine-1-yl, pyrrolidine-1-yl, imidazole-1-yl and piperazine-1-yl; and
A′ is an amino, N—(C₁-C₇-alkyl)amino, N,N-di(C₁-C₇-alkyl)amino, optionally substituted aryl, heterocyclic or heteroaryl selected from the group consisting of morpholine-4-yl, piperidine-1-yl, pyrrolidine-1-yl, imidazole-1-yl and piperazine-1-yl; or
A′ is represented by structure IIIb; 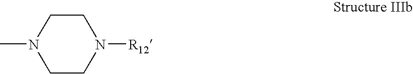
where R₁₂′ denotes hydrogen or an optionally substituted C₁-C₇ alkyl or C₂-C₇ alkenyl, C₂-C₇ alkynyl group, an optionally substituted aryl, heteroaryl or heterocyclic group, C₁-C₇ alkoxy, C₁-C₇ alkylthiol, C₁-C₇ alkanoyl, aroyl, oxo-C₁-C₇ alkyl, C₁-C₇ alkanoyloxy, carboxy, an optionally substituted C₁-C₇ alkyloxycarbonyl or aryloxycarbonyl, carbamoyl, N—(C₁-C₇-alkyl)carbamoyl, N,N-di(C₁-C₇-alkyl)carbamoyl, cyano-C₁-C₇ alkyl, C₁-C₇ alkylsulfonyl, C₁-C₇ alkylsulfinyl;
n denotes an integer from 0 to 5;

R₁₀′ denotes hydrogen or an optionally substituted C1-C7 alkyl or C2-C7 alkenyl, C₂-C₇ alkynyl group, an optionally substituted aryl, heteroaryl or heterocyclic group, C₁-C₇ alkoxy, C₁-C₇ alkylthiol, C₁-C₇ alkanoyl, aroyl, oxo-C₁-C₇ alkyl, C₁-C₇ alkanoyloxy, carboxy, an optionally substituted C₁-C₇ alkyloxycarbonyl or aryloxycarbonyl, arylalkyl, carbamoyl, N—(C₁-C₇-alkyl)carbamoyl, N,N-di(C₁-C₇-alkyl)carbamoyl, cyano-C₁-C₇ alkyl, C₁-C₇ alkylsulfonyl, C₁-C₇ alkylsulfinyl;

R₁₁′ denotes hydrogen or an optionally substituted C₁-C₇ alkyl or C₂-C₇ alkenyl, C₂-C₇ alkynyl group, an optionally substituted aryl, heteroaryl or heterocyclic group, C₁-C₇ alkoxy, C₁-C₇ alkylthiol, C₁-C₇ alkanoyl, aroyl, oxo-C₁-C₇ alkyl, C₁-C₇ alkanoyloxy, arylalkyl, carboxy, an optionally substituted C₁-C₇ alkyloxycarbonyl or aryloxycarbonyl, carbamoyl, N—(C₁-C₇-alkyl)carbamoyl, N,N-di(C₁-C₇-alkyl)carbamoyl, cyano-C₁-C₇ alkyl, C₁-C₇ alkylsulfonyl, C₁-C₇ alkylsulfinyl; as well as pharmacologically acceptable esters, salts and solvates thereof.

The linkage site with L is at any dibenzo[e,h]azulene position among R₁′-R₉′;preferably at position R₉′.

Bold-faced bonds in formulas contained herein denote bonds raised above the paper level; dash-drawn bonds denote bonds below the paper level, whereas broken lines represent a bond that may be either below or above the paper level. Parallel full and broken lines represent either a single or a double bond. Unless explicitly stated elsewhere herein, the following terms have the meanings ascribed to them below:

“Alkyl” means a linear or branched saturated monovalent hydrocarbon radical of one to ten carbon atoms, more preferably one to six carbon atoms. The preferred straight-chain or branched-chain alkyls include methyl, ethyl, propyl, iso-propyl, butyl, sec-butyl and tert-butyl. Methyl is most preferred. Alkyl groups may be substituted with one up to five substituents including halogen (preferably fluorine or chlorine), hydroxy, alkoxy (preferably methoxy or ethoxy), acyl, acylamino cyano, amino, N—(C1-C4)alkylamino (preferably N-methylamino or N-ethylamino), N,N-di(C1-C4-alkyl)amino (preferably dimethylamino or diethylamino), aryl (preferably phenyl) or heteroaryl, thiocarbonylamino, acyloxy, amino, amidino, alkyl amidino, thioamidino, aminoacyl, aminocarbonylamino, aminothiocarbonylamino, aminocarbonyloxy, aryl, heteroaryl, aryloxy, aryloxyaryl, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, cycloalkoxy, heteroaryloxy, heterocyclyloxy, and oxycarbonylamino. Such substituted alkyl groups are within the present definition of “alkyl.” The present definition of alkyl carries over to other groups having an alkyl moiety such as alkoxy.

“Alkenyl” means a linear or branched monovalent hydrocarbon radical of two to ten and preferably two to six carbon atoms which has at least one double carbon-carbon bond. Alkenyl groups may be substituted with the same groups as alkyl and such optionally substituted alkenyl groups are encompassed within the term “alkenyl.” Ethenyl, propenyl, butenyl and cyclohexenyl are preferred.

“Alkynyl” means a linear or branched monovalent hydrocarbon radical, having a straight-chain or a branched-chain of two to ten, and preferably two to six carbon atoms and containing at least one and preferably no more than three triple carbon-carbon bonds. Alkynyl groups can be substituted with the same groups as alkyl, and the substituted groups are within the present definition of alkynyl. Ethynyl, propynyl and butynyl groups are preferred.

“Alkoxy” means a linear or branched chain C₁₋₁₀ alkyl group, as previously defined, attached to the parent molecular moiety through an oxygen atom containing the specified number of carbon atoms. For example, C1-4 alkoxy means a straight or branched alkoxy containing at least 1, and at most 4, carbon atoms. Examples of “alkoxy” as used herein include, but are not limited to, methoxy, ethoxy, propoxy, prop-2-oxy, butoxy, but-2-oxy, 2-methylprop-1-oxy and 2-methylprop-2-oxy.

“Cycloalkyl” means a cyclic group having 3-8 carbon atoms having a single ring optionally fused to an aryl or heteroaryl group. The cycloalkyl groups can be substituted as specified for “aryl” below, and the substituted cycloalkyl groups are within the present definition of “cycloalkyl”. Preferred cycloalkyls are cyclopentyl and cyclohexyl.

“Aryl” means an unsaturated aromatic carbocyclic group having 6-14 carbon atoms having a single ring such as phenyl or multiple fused rings such as naphthyl. Aryl may optionally be further fused to an aliphatic or aryl group or can be substituted with one or more substituents such as halogen (fluorine, chlorine and/or bromine), hydroxy, C₁-C₇ alkyl, C₁-C₇ alkoxy or aryloxy, C₁-C₇ alkylthio or arylthio, alkylsulfonyl, cyano or primary or nonprimary amino.

“Heteroaryl” means a monocyclic or a bicyclic aromatic hydrocarbon ring having from 2 to 10 carbon atoms and from 1 to 4 heteroatoms, such as O, S or N. The heteroaryl ring may optionally be fused to another heteroaryl, aryl or aliphatic cyclic group. Examples of this type are furan, thiophene, pyrrole, imidazole, indole, pyridine, oxazole, thiazole, pyrrole, pyrazole, tetrazole, pyrimidine, pyrazine and triazine, with furan, pyrrole, pyridine and indole being preferred. The term includes groups that are substituted with the same substituents as specified for aryl above.

“Heterocyclic” means a saturated or unsaturated group having a single or multiple rings and from 1 to 10 carbon atoms and from 1-4 heteroatoms selected from nitrogen, sulphur or oxygen, wherein in a fused ring system the other ring or rings can be aryl or heteroaryl. Heterocyclic groups can be substituted as specified for alkyl groups and the thus substituted heterocyclic groups are within the present definition.

Aryl, heteroaryl or heterocycle may be optionally additionally substituted with one, two or more substituents. The substituents may be halo (chlorine or fluorine), C₁-C₄ alkyl (preferably methyl, ethyl or isopropyl), trifluoromethyl, cyano, nitro, hydroxy, C₁-C₄ alkoxy (preferably methoxy or ethoxy), C₁-C₄ alkyloxycarbonyl (preferably methyloxycarbonyl) thiol, C₁-C₄ alkylthio (preferably methylthio or ethylthio), amino, N—(C₁-C₄) alkylamino (preferably N-methylamino or N-ethylamino), N,N-di(C₁-C₄-alkyl)-amino (preferably N,N-dimethylamino or N,N-diethylamino), sulfonyl, C₁-C₄ alkylsulfonyl (preferably methylsulfonyl or ethylsulfonyl), sulfinyl, C₁-C₄ alkylsulfinyl (preferably methylsulfinyl).

The term “optionally substituted alkyl” relates to alkyl groups which may be optionally additionally substituted with one, two, three or more substituents. Such substituents may be halogen atom (preferably fluorine or chlorine), hydroxy, C₁-C₄ alkoxy (preferably methoxy or ethoxy), thiol, C₁-C₄ alkylthio (preferably methylthio or ethylthio), amino, N—(C₁-C₄)alkylamino (preferably N-methylamino or N-ethylamino), N,N-di(C₁-C₄-alkyl)-amino (preferably dimethylamino or diethylamino), sulfonyl, C₁-C₄ alkylsulfonyl (preferably methylsulfonyl or ethylsulfonyl), sulfinyl, C₁-C₄ alkylsulfinyl (preferably methylsulfinyl).

The present invention also encompasses pharmaceutically acceptable salts of the present compounds. Pharmaceutically suitable salts of the compounds of the present invention include salts with inorganic acids (e.g. hydrochloric, hydrobromic, phosphoric, metaphosphoric, nitric or sulfuric acid) or organic acids (e.g. tartaric, acetic, methane-sulfonic, trifluoroacetic, citric, maleic, lactic, fumaric, benzoic, succinic, methanesulfonic, oxalic and p-toluenesulfonic acids).

The present invention also encompasses prodrugs of the Formula I compounds, i.e., compounds which release an active parent hybrid drug according to Formula (I) in vivo when administered to a mammalian subject. Prodrugs of a compound of Formula I are prepared by modifying functional groups present in the compound of Formula I in such a way that the modifications may be cleaved in vivo to release the parent compound. Prodrugs include compounds of Formula I wherein a hydroxy, amino, or carboxy group of a Formula I compound is bonded to any group that may be cleaved in vivo to regenerate the free hydroxyl, amino or carboxy group, respectively. Examples of prodrugs include, but are not limited to esters (e.g., acetate, formate, and benzoate derivatives) of compounds of Formula I, or any other derivative which upon being brought to the physiological pH or through enzyme action is converted to the active parent drug.

The present invention also encompasses solvates (preferably hydrates) of the compounds of Formula I or their salts.

The compounds of the Formula I may have one or more chirality centers and, depending on the nature of individual substituents, they can also comprise geometrical isomers. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has a chiral center, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomer respectively). A chiral compound can exist as either an individual enantiomer or as a mixture of enantiomers. A mixture containing equal proportions of the enantiomers is called a “racemic mixture”. The present invention encompasses all individual isomers of compounds of Formula I. The description or naming of a particular compound in the specification and claims is intended to include both individual enantiomers and mixtures, racemic or otherwise, thereof. Methods for the determination of stereochemistry and the resolution of stereoisomers are well-known in the art.

The present invention also encompasses stereoisomers of the syn-anti type, and mixtures thereof encountered when an oxime or similar group is present. The group of highest Cahn Ingold Prelog priority attached to one of the terminal doubly bonded atoms of the oxime, is compared with hydroxyl group of the oxime. The stereoisomer is designated as Z (zusammen=together) or Syn if the oxime hydroxyl lies on the same side of a reference plane passing through the C═N double bond as the group of highest priority; the other stereoisomer is designated as E (entgegen=opposite) or Anti.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

“Treating” or “treatment” of a state, disorder or condition includes:

(1) preventing or delaying the appearance of at least one clinical symptom of the state, disorder or condition developing in a mammal that may be afflicted with or predisposed to the state, disorder or condition but does not yet experience or display clinical or subclinical symptoms of the state, disorder or condition,

(2) inhibiting the state, disorder or condition, i.e., arresting or reducing the development of the disease or at least one clinical or subclinical symptom thereof, or

(3) relieving or attenuating the disease, i.e., causing regression of the state, disorder or condition or at least one of its clinical or subclinical symptoms.

The benefit to a subject to be treated is either statically significant or at least perceptible to the patient or to the physician.

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The four classic symptoms of acute inflammation are redness, elevated temperature. Swelling, and pain in the affected area, and loss of function of the affected organ.

Symptoms and signs of inflammation associated with specific conditions include:
rheumatoid arthritis-pain, swelling, warmth and tenderness of the involved joints; generalized and morning stiffness;
insulin-dependent diabetes mellitus-insulitis; this condition can lead to a variety of complications with an inflammatory component, including:
retinopathy, neuropathy, nephropathy; coronary artery disease, peripheral vascular disease, and cerebrovascular disease;
autoimmune thyroiditis-weakness, constipation, shortness of breath, puffiness of the face, hands and feet, peripheral edema, bradycardia;
multiple sclerosis-spasticity, blurry vision, vertigo, limb weakness, paresthesias;
uveoretinitis-decreased night vision, loss of peripheral vision;
lupus erythematosus-joint pain, rash, photosensitivity, fever, muscle pain, puffiness of the hands and feet, abnormal urinalysis (hematuria, cylinduria, proteinuria), glomerulonephritis, cognitive dysfunction, vessel thrombosis, pericarditis;
scleroderma-Raynaud's disease; swelling of the hands, arms, legs and face; skin thickening; pain, swelling and stiffness of the fingers and knees, gastrointestinal dysfunction, restrictive lung disease; pericarditis; renal failure;
other arthritic conditions having an inflammatory component such as rheumatoid spondylitis, osteoarthritis, septic arthritis and polyarthritis-fever, pain, swelling, tenderness;
other inflammatory brain disorders, such as meningitis, Alzheimer's disease, AIDS dementia encephalitis-photophobia, cognitive dysfunction, memory loss;
other inflammatory eye inflammations, such as retinitis-decreased visual acuity;
inflammatory skin disorders, such as, eczema, other dermatites (e.g., atopic, contact), psoriasis, burns induced by UV radiation (sun rays and similar UV sources)—erythema, pain, scaling, swelling, tenderness;
inflammatory bowel disease, such as Crohn's disease, ulcerative colitis-pain, diarrhea, constipation, rectal bleeding, fever, arthritis;
asthma-shortness of breath, wheezing;
other allergy disorders, such as allergic rhinitis-sneezing, itching, runny nose
conditions associated with acute trauma such as cerebral injury following stroke-sensory loss, motor loss, cognitive loss;
heart tissue injury due to myocardial ischemia-pain, shortness of breath;
lung injury such as that which occurs in adult respiratory distress syndrome-shortness of breath, hyperventilation, decreased oxygenation, pulmonary infiltrates;
inflammation accompanying infection, such as sepsis, septic shock, osteomyclitis, toxic shock syndrome-fever, respiratory failure, tachycardia, hypotension, leukocytosis;
other inflammatory conditions associated with particular organs or tissues, such as nephritis (e.g., glomerulonephritis)-oliguria, abnormal urinalysis;
inflamed appendix-fever, pain, tenderness, leukocytosis;
gout-pain, tenderness, swelling and erythema of the involved joint, elevated serum and/or urinary uric acid;
inflamed gall bladder-abdominal pain and tenderness, fever, nausea, leukocytosis;
chronic obstructive pulmonary disease-shortness of breath, wheezing;
congestive heart failure-shortness of breath, rales, peripheral edema, chronic sinusitis, nasal polyps, cystic fibrosis, diffuse panbronchiolitis, bronchiectasis, bronchiolitis obliterans;
Type II diabetes-end organ complications including cardiovascular, ocular, renal, and peripheral vascular disease;
lung fibrosis-hyperventilation, shortness of breath, decreased oxygenation;
vascular disease, such as coronary artery disease, atherosclerosis and restenosis-pain, loss of sensation, diminished pulses, loss of function; and
alloimmunity leading to transplant rejection-pain, tenderness, fever.

Subclinical symptoms include without limitation diagnostic markers for inflammation the appearance of which may precede the manifestation of clinical symptoms. One class of subclinical symptoms is immunological symptoms, such as the invasion or accumulation in an organ or tissue of proinflammatory lymphoid cells or the presence locally or peripherally of activated pro-inflammatory lymphoid cells recognizing a pathogen or an antigen specific to the organ or tissue. Activation of lymphoid cells can be measured by techniques known in the art.

“Delivering” a therapeutically effective amount of an active ingredient to a particular location within a host means causing a therapeutically effective blood concentration of the active ingredient at the particular location. This can be accomplished, e.g., by local or by systemic administration of the active ingredient to the host.

Preferably, in the compounds represented by the Formula II, Z and W together are —N(R_{N})C(O)—, —C(O)N(R_{N}). —, >C—NRₛRₜ, —C(O)—, >C═N—R_{M}, —CH₂NR_{N}— or —NR_{N}CH₂—, most preferably, —NCH₃CH₂—, —NHCH₂—, —CH₂NH—, —C(O)NH, —NHCO—,

Rₛ, Rₜ is methyl or H;

R_{M} is OH or methoxy;

X is O;

R_{N} is H, methyl, or —C(═X)—NRₜRₛ;

A is H or methyl

U, Y are H, F, methyl or hydroxymethyl;

R¹ is hydroxy, —O—S², or ═O

R² is H, hydroxy or methoxy;

R³ is OH, methoxy or a group that forms a cyclic carbamate bridge with W or Z;

R⁴ is methyl;

R⁵ is H, OH, methoxy or a group that forms a cyclic carbonate or carbamate bridge with R³;

The linkage is through the nitrogen of Z at N/9a or N/8a positions of the macrolide or through the carbon of R¹² or through the oxygen of R¹¹ both at C/4″ position of S² sugar.

R⁶ is H, methyl or ethyl;

R⁹ is H, N(CH₃)₂, NH(CH₃) or N(CH₃)CH₂CH₃,

R⁹ is H

The linkage site is preferably at position C/3; or through the amino group at position C/3′ of S¹ sugar or at position C/11 or at W or Z, or through position C/4″ of S2 sugar.

Also preferred are compounds within Formula I wherein M is of Formula II and (i) Z is NCH₃, W is CH₂, R² is hydroxy; or (ii) Z is NH, W is ═CO, and R² is methoxy. (The compounds described in this paragraph may or may not satisfy the remaining foregoing preferences in the immediately preceding section, but preferably they do.)

A further aspect of the present invention relates to processes for the preparation of compounds represented by Formula I. Generally, the compounds of Formula I may be obtained in the following way: one end of the chain L is first linked to the macrolide subunit M, and then the other end of the chain is linked to the dibenzo[e,h]azulene subunit D; or, one end of the chain L is first linked to the dibenzo[e,h]azulene subunit D and then the other end of the chain to the macrolide subunit M, or finally, one portion of the chain is linked to the macrolide subunit M, whereas the other portion of the chain is linked to the dibenzo[e,h]azulene subunit D, with the ends of the chain parts being then chemically linked to form the chain L.

It will be appreciated by those skilled in the art that it may be desirable to use protected derivatives of intermediates used in the preparation of the compounds of Formula I. Protection and deprotection of functional groups may be performed by methods known in the art. Hydroxyl or amino groups may be protected with any hydroxyl or amino protecting group, for example, as described in Green T. W.; Wuts P. G. M. Protective Groups in Organic Synthesis: John Wiley and Sons, New York, 1999. The amino protecting groups may be removed by conventional techniques. For example, acyl groups, such as alkanoyl, alkoxycarbonyl and aroyl groups, may be removed by solvolysis, e.g., by hydrolysis under acidic or basic conditions. Arylmethoxycarbonyl groups (e.g., benzyloxycarbonyl) may be cleaved by hydrogenolysis in the presence of a catalyst such as palladium-on-charcoal.

More specifically, compounds within Formula I can be prepared by the following processes.
a) Compounds of Formula I, where X² is —NH—, can be formed by reacting a dibenzo[e,h]azulene subunit represented by Formula V: 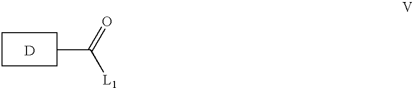
wherein L₁ represents a leaving group (such as hydroxy), and a free amino group of a macrolide subunit represented by Formula VIa:

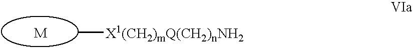

The reaction is generally performed with by prior conversion of the carboxylic acid of the nonsteroidal anti-inflammatory subunit into an activated derivative, such as a halogenide, a mixed anhydride, or a reaction of the carboxylic acid with a carbodiimide (such as -(3-dimethylaminopropyl)-3-ethyl-carbodiimide (EDC) and benzotriazoles) in situ. The reaction proceeds in the presence of a base, such as an organic base (e.g., triethylamine), at room temperature and under an inert atmosphere, such as nitrogen or argon. The reaction may require several hours to several days to reach full conversion.

For example, when L is —K—NH— (wherein K is the portion of the linking molecule L attached to the macrolide) the compound of Formula I can be formed by derivatizing an >NH group on the macrolide ring to an >N—K—NH₂ group and reacting the derivatized macrolide with a dibenzo[e,h]azulene subunit represented by Formula V; wherein L¹ is a leaving group according to Scheme I.

All dibenzoazulene subunits including ones represented by Formula V are synthesized according to patent applications WO 03/097648, WO 03/097649, WO 03/099823, WO 03/099827, WO 03/084964 and WO 01/87890, each incorporated by reference in its entirety.

Preparation of the Starting Macrolide Subunits of the Structure VIa has been described in PCT WO 02/055531 A1, incorporated by reference in its entirety. See also Bright, U.S. Pat. No. 4,474,768 and Bright, G. M. et al. J. Antibiot. 1988, 41, 1029-1047. each incorporated by reference in its entirety.

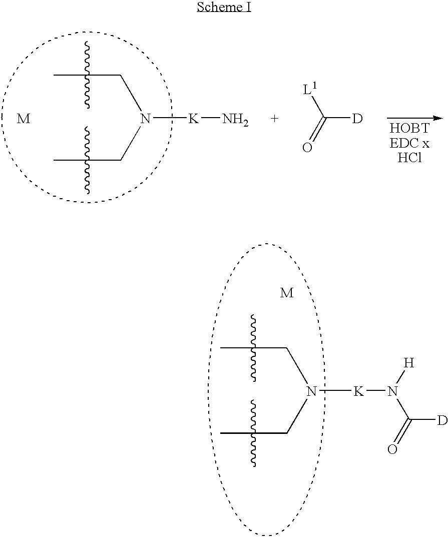

This process may also be performed when the NH group in the macrolide is attached at the 3′ position of a sugar ring S¹ (i.e., desozamine sugar when R^{Z} is hydrogen) of the macrolide according to Scheme II:

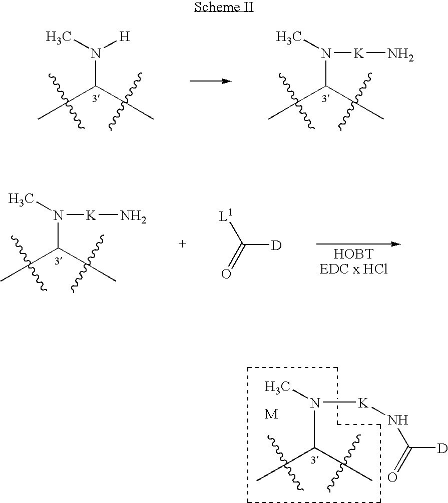

or the 4″ position of the sugar ring S² (i.e., a cladinose sugar when R¹¹ is hydrogen) according to Scheme III:

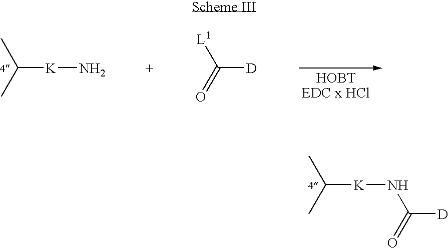

The reactant macrolide subunit can be formed by oxidizing the corresponding macrolide having a hydroxy substituent at the 4″ position on cladinose sugar to obtain a ═O substituent at the 4″ position, converting the 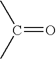
at the 4″ position to an epoxy group ( 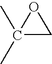
), and cleaving the epoxy group with an appropriate reactant(s) to yield the reactant macrolide subunit (M-CH₂—NH—K—NH₂).
b) Compounds represented by Formula I, where X² is —OC(O)—, can be formed by reacting a dibenzo[e,h]azulene subunit represented by Formula V and the free hydroxyl group of a macrolide subunit represented by Formula VIb:

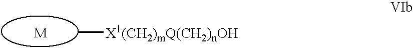

The reaction is generally performed by conversion of the carboxylic acid of the nonsteroidal anti-inflammatory subunit into an activated derivative, such as a halogenide, a mixed anhydride, or a reaction of the carboxylic acid with a carbodiimide (such as -(3-dimethylaminopropyl)-3-ethyl-carbodiimide (EDC) and benzotriazoles) in situ. The reaction is typically performed at room temperature under an inert atmosphere, such as nitrogen or argon. The reaction may require several hours to several days to come to completion.

The starting macrolide subunits of the structure VIb are known compounds or may be obtained according to the procedures described for analogous compounds, such as those described in Costa A. M. et al. Tetrahedron Letters 2000, 41, 3371-3375, which is hereby incorporated by reference.

For example, when linkage L is —K—O—, the compound of Formula I can be formed by (1) derivatizing an >NH group on a macrolide to an >N—K—OH group and (2) reacting the derivatized macrolide with the free carboxylic acid group on a dibenzo[e,h]azulene anti-inflammatory subunit D according to Scheme IV:

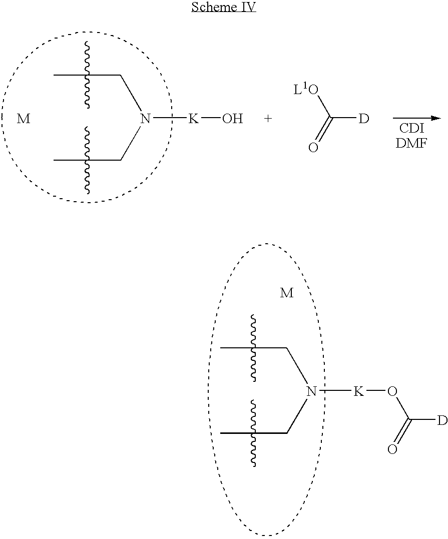

The linkage group —K—OH can be attached to the primary or secondary nitrogen atom of the macrolide subunit as follows. The macrolide subunit is reacted with an alkenoyl derived Michael acceptor, such as CH₂═CH(CH₂)ₘC(O)O-Alkyl (e.g., methylacrylate). The ester group (i.e., —C(O)O-Alkyl) is then reduced, such as with a metal hydride (e.g., LiAlH₄) in an anhydrous organic solvent, to yield the macrolide subunit having the linkage group —K—OH (i.e., M-K—OH). The reduction is typically performed at a low temperature and preferably at 0° C. or lower.

This process can also be performed when the NH group is attached at the 3′ position of a sugar ring in the macrolide (such as a sugar at the 5 position of the macrolide).
c) Compounds represented by Formula I, wherein X¹ is —OC(O)—, Q is —CH₂— or NH, and X² is —NH—, can be prepared by reacting a macrolide subunit represented by the formula 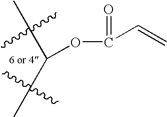
   where 4″ is the 4 position on a sugar S², such as a cladinose sugar, and a derivatized dibenzo[e,h]azulene subunit having a free amino group represented by the formula: 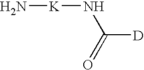
   in a solvent, such as acetonitrile, to yield a Michael product as shown below.

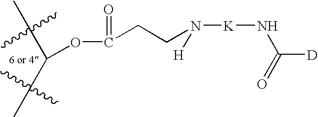

The derivatized dibenzo[e,h]azulene subunit (i.e., D-C(O)—NH—K—NH₂) may be formed by reacting an appropriate amine (having the linkage group —K—NH₂) with a carboxylic acid group of a dibenzo[e,h]azulene subunit.
d) Compounds represented by Formula I, where X¹ is —OC(O)NH— and X² is —NH—, can be prepared by reacting a macrolide subunit and a derivatized dibenzo[e,h]azulene subunit having a free amino group as shown below. 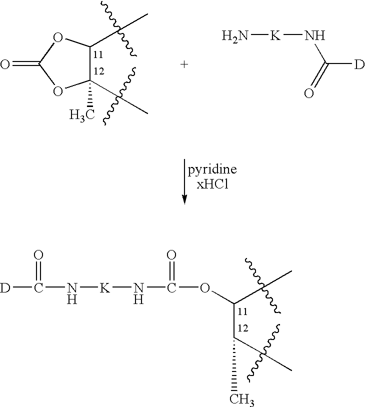
e) Compounds represented by Formula I, where X¹ is —OC(O)NH— and X² is —NH—, can be also prepared by reacting a macrolide subunit and a dibenzo[e,h]azulene subunit having a free carboxylic acid group as shown below. 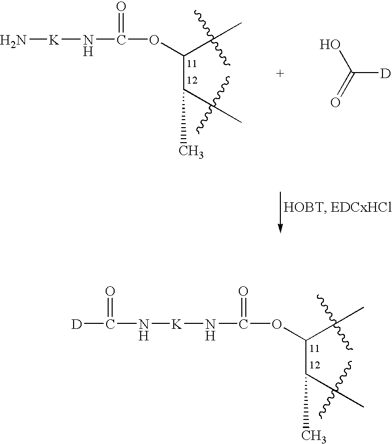
f) The compounds of the Formula I can be prepared by reacting a macrolide subunit having a leaving group L² (such as Br), and dibenzo[e,h]azulene subunits as shown below. 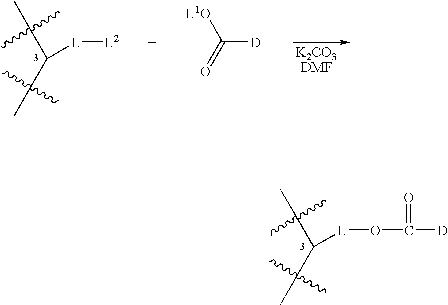
The starting macrolide subunit can be prepared by cleaving the sugar group attached at the 3-position of the macrolide ring and then reacting the macrolide with a reagent of the Formula L²-L-L¹, where L² is a leaving group.
g) The compounds of Formula I can be prepared by reacting a macrolide subunit having a leaving group L² (such as Br), and dibenzo[e,h]azulene subunit as shown below. 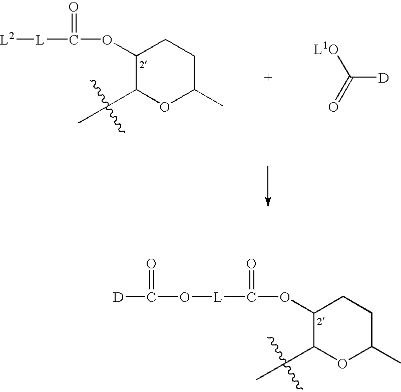
h) Compounds of the Formula I can be prepared by reacting a macrolide subunit having a leaving group L² (such as Br) and a dibenzo[e,h]azulene subunit as shown below.

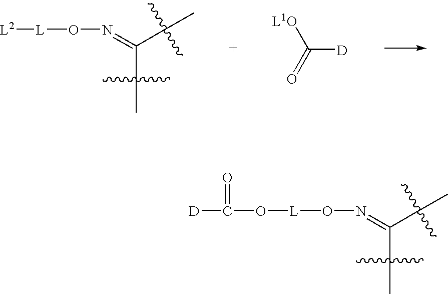

The 16-membered ring macrolides are traditionally divided into sub-families based upon the substitution patterns of their aglycones. The principal prototypes of this family can be represented by leucomycin, spiramycin and tylosin.

Tylosin is a representative of 16-membered macrolides, which possesses a highly substituted aglycone with two double bonds (tylonolide) and a third saccharide substituent (β-D-mycinose) beta-D-mycosine in addition to the disaccharide attached to the 5-hydroxyl group. Hydrolysis of mycarose from disaccharide yielded desmycarosyl-tylosin (desmycosin).
Potential sites of modification in desmycosin: 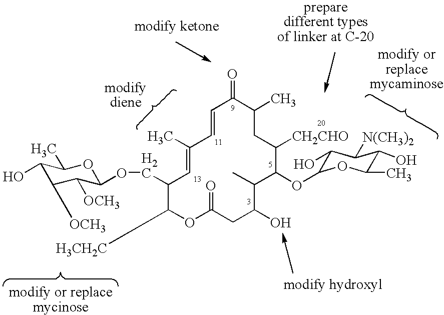
For example, a 16-membered ring macrolide hybrid could be prepared by reductive amination of the C-20 aldehyde group.

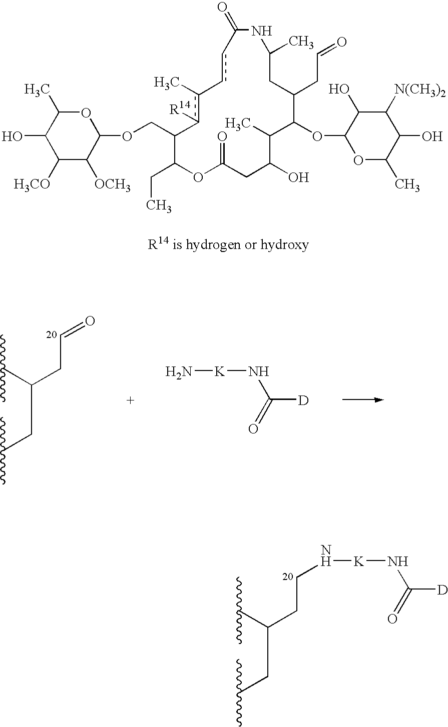

This reaction could be used also for 17-membered azalides like 8a-aza-homodesmycosins and its derivatives (such as di- and tetrahydro derivatives). Other possibilities in 16-membered ring macrolide derivatisation are transformations of double bonds by epoxidation, and cleaving the epoxy group with an appropriate reactant (such as a diamine) to yield the reactant macrolide subunit (M-CH₂—NH—K—NH₂).

Also the ketone in position 9 can be modified by hydroxylamine hydrochloride to yield the corresponding oxime and then reduced to amine.

A further aspect of the present invention relates to the use of compounds of Formula I in the abatement of inflammation and in the treatment of inflammatory diseases, disorders and conditions characterized by or associated with an undesirable inflammatory immune response, especially of all diseases and conditions induced by or associated with an excessive secretion of TNF-α and IL-1.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

Broad and preferred effective amounts of the compound, a pharmaceutically salt thereof, a solvate thereof, or a prodrug thereof are shown in the table below.

| | Amount of Compound, Pharmaceutically Salt Thereof, |
|---|---|
| | Solvate Thereof, or Prodrug Thereof |
| | μmol/kg body weight/day of the hybrid |
| Broad | from about 0.004 to about 4000 |
| Preferred | from about 0.04 to about 400 |
| More Preferred | from about 4 to about 400 |
| Most Preferred | from about 12 to about 120 |

Further, the present invention relates to pharmaceutical compositions containing an effective dose of compounds of the present invention as well as pharmaceutically acceptable excipients, such as carriers or diluents.

Efficacy of the present compounds can be assessed by any method for assessing inflammation or anti-inflammatory effect. There are many known methods for this purpose including without limitation, use of contrast ultrasound in conjunction with injection of microbubbles, measurement of inflammatory cytokines (such as TNF-α, IL-1, IFN-γ, IL-6, IL-8, IL2, and IL-5) measurement of activated immune system cells as well as observation (reduction of oedema, reduction of erythema, reduction of pruritus or burning sensation, reduction of body temperature, improvement in function of the afflicted organ) as well as any of the methods provided below.

Pharmaceutical Compositions

Further, the present invention relates to pharmaceutical compositions containing an effective dose of compounds of the present invention as well as pharmaceutically acceptable excipients, such as carriers or diluents.

While it is possible that, for use in the methods of the invention, a compound of formula I may be administered as the bulk substance, it is preferable to present the active ingredient in a pharmaceutical formulation, e.g., wherein the agent is in admixture with a pharmaceutically acceptable carrier selected with regard to the intended route of administration and standard pharmaceutical practice.

The corresponding preparations of the compounds of the present invention can be used in the prophylaxis (including without limitation the prevention, delay or inhibition of recurrence of one or more of the clinical or subclinical symptoms discussed and defined in connection with the definitions of “treatment” above, as well as in the therapeutic treatment of several diseases and pathological inflammatory conditions including: chronic obstructive pulmonary disorder (COPD), asthma, inflammatory nasal diseases such as allergic rhinitis, nasal polyps, intestinal diseases such as Crohn's disease, colitis, intestinal inflammation, ulcerative colitis, dermatological inflammations such as eczema, psoriasis, allergic dermatitis, neurodermatitis, pruritis, conjunctivitis and rheumatoid arthritis.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition. The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

It will be appreciated that pharmaceutical compositions for use in accordance with the present invention may be in the form of oral, parenternal, transdermal, inhalation, sublingual, topical, implant, nasal, or enterally administered (or other mucosally administered) suspensions, capsules or tablets, which may be formulated in conventional manner using one or more pharmaceutically acceptable carriers or excipients.

There may be different composition/formulation requirements depending on the different delivery systems. It is to be understood that not all of the compounds need to be administered by the same route. Likewise, if the composition comprises more than one active component, then those components may be administered by the same or different routes. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

The present invention further relates to pharmaceutical formulations containing a therapeutically effective quantity of a compound of formula I or one of its salts mixed with a pharmaceutically acceptable vehicle. The pharmaceutical formulations of the present invention can be liquids that are suitable for oral, mucosal and/or parenteral administration, for example, drops, syrups, solutions, injectable solutions that are ready for use or are prepared by the dilution of a freeze-dried product but are preferably solid or semisolid as tablets, capsules, granules, powders, pellets, pessaries, suppositories, creams, salves, gels, ointments; or solutions, suspensions, emulsions, or other forms suitable for administration by the transdermal route or by inhalation.

The compounds of the invention can be administered for immediate-, delayed-, modified-, sustained-, pulsed- or controlled-release applications.

The compound can also be incorporated into a formulation for treating inflammation localized in an organ or tissue, e.g., Crohn's disease, where it can be administered orally or rectally. Formulations for oral administration can incorporate excipients enabling bioavailability of the compound at the site of inflammation. This can be achieved by different combinations of enteric and delayed release formulations. The compound of Formula I can also be used in the treatment of Crohn's disease and intestinal inflammation disease if the compound is applied in the form of a clyster, for which a suitable formulation can be used, as is well known in the field.

In some embodiments, the oral compositions are slow, delayed or positioned release (e.g., enteric especially colonic release) tablets or capsules. This release profile can be achieved without limitation by use of a coating resistant to conditions within the stomach but releasing the contents in the colon or other portion of the GI tract wherein a lesion or inflammation site has been identified. Or a delayed release can be achieved by a coating that is simply slow to disintegrate. Or the two (delayed and positioned release) profiles can be combined in a single formulation by choice of one or more appropriate coatings and other excipients. Such formulations constitute a further feature of the present invention.

Formulations for oral administration can be so designed to enable bioavailability of the compound at the site of inflammation in the intestines. This can be achieved by different combinations of delayed release formulations. The compound of Formula I can also be used in the treatment of Crohn's disease and intestinal inflammation disease if the compound is applied in the form of an enema, for which a suitable formulation can be used.

Suitable compositions for delayed or positioned release and/or enteric coated oral formulations include tablet formulations film coated with materials that are water resistant, pH sensitive, digested or emulsified by intestinal juices or sloughed off at a slow but regular rate when moistened. Suitable coating materials include, but are not limited to, hydroxypropyl methylcellulose, ethyl cellulose, cellulose acetate phthalate, polyvinyl acetate phthalate, hydroxypropyl methylcellulose phthalate, polymers of metacrylic acid and its esters, and combinations thereof. Plasticizers such as, but not limited to polyethylene glycol, dibutylphthalate, triacetin and castor oil may be used. A pigment may also be used to color the film. Suppositories are be prepared by using carriers like cocoa butter, suppository bases such as Suppocire C, and Suppocire NA50 (supplied by Gattefosse Deutschland GmbH, D-Weil am Rhein, Germany) and other Suppocire type excipients obtained by interesterification of hydrogenated palm oil and palm kernel oil (C8-C18 triglycerides), esterification of glycerol and specific fatty acids, or polyglycosylated glycerides, and whitepsol (hydrogenated plant oils derivatives with additives). Enemas are formulated by using the appropriate active compound according to the present invention and solvents or excipients for suspensions. Suspensions are produced by using micronized compounds, and appropriate vehicle containing suspension stabilizing agents, thickeners and emulsifiers like carboxymethylcellulose and salts thereof, polyacrylic acid and salts thereof, carboxyvinyl polymers and salts thereof, alginic acid and salts thereof, propylene glycol alginate, chitosan, hydroxypropylcellulose, hydroxypropylmethylcellulose, hydroxyethylcellulose, ethylcellulose, methylcellulose, polyvinyl alcohol, polyvinyl pyrolidone, N-vinylacetamide polymer, polyvinyl methacrylate, polyethylene glycol, pluronic, gelatin, methyl vinyl ether-maleic anhydride copolymer, soluble starch, pullulan and a copolymer of methyl acrylate and 2-ethylhexyl acrylate lecithin, lecithin derivatives, propylene glycol fatty acid esters, glycerin fatty acid esters, sorbitan fatty acid esters, polyoxyethylene sorbitan fatty acid esters, polyethylene glycol fatty acid esters, polyoxyethylene hydrated caster oil, polyoxyethylene alkyl ethers, and pluronic and appropriate buffer system in pH range of 6.5 to 8. The use of preservatives, masking agents is suitable. The average diameter of micronized particles can be between 1 and 20 micrometers, or can be less than 1 micrometer. Compounds can also be incorporated in the formulation by using their water-soluble salt forms.

Alternatively, materials may be incorporated into the matrix of the tablet e.g. hydroxypropyl methylcellulose, ethyl cellulose or polymers of acrylic and metacrylic acid esters. These latter materials may also be applied to tablets by compression coating.

Pharmaceutical compositions can be prepared by mixing a therapeutically effective amount of the active substance with a pharmaceutically acceptable carrier that can have different forms, depending on the way of administration. Pharmaceutical compositions can be prepared by using conventional pharmaceutical excipients and methods of preparation. The forms for oral administration can be capsules, powders or tablets where usual solid vehicles including lactose, starch, glucose, methylcellulose, magnesium stearate, di-calcium phosphate, mannitol may be added, as well as usual liquid oral excipients including, but not limited to, ethanol, glycerol, and water. All excipients may be mixed with disintegrating agents, solvents, granulating agents, moisturizers and binders. When a solid carrier is used for preparation of oral compositions (e.g., starch, sugar, kaolin, binders disintegrating agents) preparation can be in the form of powder, capsules containing granules or coated particles, tablets, hard gelatin capsules, or granules without limitation, and the amount of the solid carrier can vary (between 1 mg to 1 g). Tablets and capsules are the preferred oral composition forms.

Pharmaceutical compositions containing compounds of the present invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyanisole.

The compounds of the invention may also, for example, be formulated as suppositories e.g., containing conventional suppository bases for use in human or veterinary medicine or as pessaries e.g., containing conventional pessary bases.

For percutaneous or mucosal external administration, the compound of Formula I can be prepared in a form of an ointment or cream, gel or lotion. Ointments, creams and gels can be formulated using a water or oil base with addition of an appropriate emulsifier or gelling agent Formulation of the present compounds is especially significant for respiratory inhalation, wherein the compound of Formula I is to be delivered in the form of an aerosol under pressure. It is preferred to micronize the compound of Formula I after it has been homogenised, e.g., in lactose, glucose, higher fatty acids, sodium salt of dioctylsulfosuccinic acid or, most preferably, in carboxymethyl cellulose, in order to achieve a microparticle size of 5 μm or less for the majority of particles. For the inhalation formulation, the aerosol can be mixed with a gas or a liquid propellant for dispensing the active substance. An inhaler or atomizer or nebulizer may be used. Such devices are known. See, e.g., Newman et al., Thorax, 1985, 40:61-676 Berenberg, M., J. Asthma USA, 1985, 22:87-92. A Bird nebulizer can also be used. See also U.S. Pat. Nos. 6,402,733; 6,273,086; and 6,228,346.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colorants, and odorants.

Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134AT″″) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebulizer.

The pharmaceutical compositions of the invention may contain from 0.01 to 99% weight per volume of the active material.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

The therapeutic effect of compounds of the present invention was determined in in vitro and in vivo experiments such as the following.

The cytokines assayed in the biological examples, when expressed at elevated amounts, are markers for inflammation and, in the case of cell proliferation, and lung eosinophilia, the behaviors of these immune cells are also markers for their activation and, therefore, inflammation. Consequently, reduction of pro-inflammatory cytokine expression (i.e., TNF-α, IL-1, IL-6, IL-8, IL-2, and IL-5) or secretion and reduction in cell proliferation, degranulation or neutrophil eosinophil accumulation is a measure of a compound's anti-inflammatory activity. Lung neutrophilia specifically serves as a model for COPD and lung eosinophilia as a model for asthma. Prostaglandins and leukotrienes (as well as 5-Lox) are also potent inflammation mediators, the former being produced in the cyclooxygenase 2 pathway and the latter in the lipooxygenase pathway.

5-Lox Inhibition Assay

RBL-2H3 cell line (ATCC 2256) is grown in DMEM medium (Invitrogen) supplemented with 10% FBS (Invitrogen) in an atmosphere of 5% CO₂, 90% humidity, and 37° C. Cells are trypsinized, washed with fresh DMEM medium, and adjusted to 1×10₅ cells per milliliter. 500 μL/well of cell suspension is transferred into 24 well plate (Falcon) and grown overnight in culturing condition described herein. 10 mM solutions of tested compounds are prepared in DMSO (Sigma), and dissolved in working concentrations in DMEM medium without phenol red (Invitrogen). Dilutions of tested compounds are introduced into wells containing cells, whereas for control samples only DMEM medium without phenol red is used. Cells and additive are incubated for 30 minutes. Calcimycin A23187 (Sigma) was added to a final concentration of 250 nM and incubated for 45 minutes. 10 μL of cellular supernatant was used to determine leukotriene B4 levels using ELISA (R&D systems). Total concentrations of leukotriene B4 (LTB4) a stimulant for 5-10× are calculated in samples, and total inhibition was calculated using the formula:
% inhibition=(1−LTB4 sample concentration/LTB4 positive control concentration)*100.

IC₅₀ value of leukotriene B4 inhibition at a 10 μM concentration or lower concentrations is a cut-off value which was used to determine the preferred in vitro inhibitors. Zileutone is used as a standard for comparison and compounds are preferred to have equal or lower IC₅₀ values than zileutone, i.e., the IC₅₀ concentration should be 10 μm or less. Compounds 1, 2, 4, 5, 28, 31, 32, 35, 38, 40, 43, 44 are among the most potent compounds with IC₅₀ values below 5 μm.

Determination of TNF-α and IL-1p Secretion in Mononuclear Cells of Human Peripheral Blood In Vitro

Peripheral blood mononuclear cells (PMBC) were obtained from heparinized whole blood after separation of PMBC on Ficoll-Hypaque (Amersham-Pharmacia). For a determination of TNF-α, 3.5−5×10⁴ cells were cultured in a total volume of 200 μL within a period of 18 to 24 hours on microtiter flat bottom plates (96 wells, Falcon) in RPMI 1640 medium supplemented with 10% of heat-inactivated human AB serum (Croatian Centre For Transfusion Medicine, Zagreb), 100 units/mL of penicillin, 100 mg/mL of streptomycin and 20 mM HEPES (Invitrogen Life Technologies). The cells were incubated at 37° C. in an atmosphere with 5% CO₂ and 90% moisture. The cells in a negative control were cultured only in the medium (NC), while the secretion of TNF-α in a positive control was stimulated by the addition of 1 μg/mL lipopolysaccharide (LPS, E. coli serotype 0111:B4, Sigma) (PC). The effect of the tested substances on TNF-α secretion was tested after their addition to cell cultures stimulated with LPS (TS). The TNF-α level in the cell supernatant was determined by ELISA according to the manufacturer's (R&D Systems) suggestions. The test sensitivity was <3 pg/mL TNF-α. The determination of IL-1β was performed as described for TNF-α determination, but 1×10⁵ cells/well and 0.1 ng/mL of LPS were used. IL-1β was determined by ELISA (R&D Systems). The percentage inhibition of TNF-α or IL-1β production was calculated by the following equation:
% inhibition=[1−(TS−NC)/(PC−NC)]×100.

IC₅₀ value was defined as the concentration of the substance at which 50% of TNF-α production was inhibited. The compounds demonstrating IC₅₀ in concentrations of 20 μM or lower were considered active. IC₅₀ was calculated using Graph Pad Prism Software.

In this assay, among the most active compounds are 3, 5, 7, 9, 10, 11, 12, 13, 15, 17, 20, 23, 24, 27, 29, 30, 34, 35, 38, 42, 43, 45, 48 with IC₅₀ values below 3 μm.

Determination of TNF-α Secretion by RAW 264.7 Cells

The cells were grown in 10% fetal bovine serum (FBS) in DMEM medium (Invitrogen Life Technologies) at 37° C. in an atmosphere with 5% CO₂ and 90% moisture. 20 000 cells/well were plated in 96 well plate (Falcon). The cells in a negative control were cultured only in medium (NC), while the secretion of TNF-α in a positive control was stimulated by the addition of 500 pg/mL lipopolysaccharide (LPS, E. coli serotype 0111:B4, Sigma) (PC). The effect of the tested substances on TNF-α secretion was assessed after their addition to cell cultures stimulated with LPS (TS). The TNF-α level in the cell supernatant was determined by ELISA according to manufacturer s (R&D Systems, Biosource) suggestions. The percentage inhibition of TNF-α production was calculated by the following equation:
% inhibition=[1−(TS−NC)/(PC−NC)]×100.
IC₅₀ value was defined as the concentration of the substance at which 50% of TNF-α production was inhibited. The compounds demonstrating IC₅₀ in concentrations of 10 μM or lower were considered active.
Human Prostaglandin-H Synthase-1 (hPGH-1) and Human Prostaglandin-H Synthase-2 (hPGH-2) Inhibition Assay

Genes coding hPGH-1 and hPGH-2 were amplified with PCR using Platinum pfx DNA polymerase (Invitrogen Life Technologies) from human placenta cDNA library (Stratagene). Primer sequences used for hPGH-1 are: 5′ ATATAAGCTTGCGCCATGAGCCGGAGTCTTC 3′ and 5′ ATATGGATCCTCAGAGCTCTGTGGATGGTCGC 3′; for hPGH-2 5′ ATATAAGCTTGCTGCGATGCTCGCCCGC 3′ and 5′ ATATGGATCCCTACAGTTCAGTTCAGTCGAACGTTC 3′. PCR products were cloned into HindIII and BamHI restriction sites of pcDNA3.1 Hygro(+) plasmid (Invitrogen Life Technologies), sequences were confirmed by sequencing.

COS-7 cells (ATCC) were transferred and grown in 10% fetal bovine serum (FBS) in DMEM medium (Invitrogen Life Tecnologies), 37° C. in an atmosphere with 5% CO₂ and 90% moisture, to full confluency in 24-well plates (Falcon). 1 μg plasmid DNA (pcDNA Hygro 3.1 (+) containing PGH-1 or PGH-2 gene, or pcDNA Hygro 3.1 (+) for negative control samples) was combined with 1.5 μl Lipofectamine 2000 (Invitrogen Life Technologies), following manufacturer's recommendations. 24-48 hours post transfection, tested compounds in DMEM were added to cells without medium removal, and after 40 minutes, arachidonic acid (Sigma) was added to final 20 μM concentration. After 30 minutes supernatants were removed and prostoglandin E2 (PGE-2) was measured with a PGE-2 assay kit (Cayman) following the manufacturer's instructions. No production of PGE-2 was detected in negative controls. % inhibition was calculated by the following equation:
% inhibition=(1-sample PGE-2 concentration/positive control PGE-2 concentration)*100.
Compound 15 inhibit COX-2 with IC₅₀ value below 10 μm.

A compound is considered to be “active” if it is better than a positive control in at least one inhibitory function (i.e., inhibition PGE-2).

In Vivo Model of LPS-Induced Exccessive Secretion of TNF-α in Mice

TNF-α secretion in mice was induced according to the previously described method (Badger A. M. Et al., J. of Pharmac. and Env. Therap. 279 1996 1453-1461). In the test, male BALB/c mice at an age of 8 to 12 weeks in groups of 6 to 10 animals were used. Animals were treated p.o. either only with the solvent and not stimulated with LPS (negative control) or with solvent and stimulated with LPS (positive control) or treated with solutions of the substance 30 minutes prior to the i.p. treatment with LPS (E. coli serotype 0111:B4, Sigma) in a dose of 25 μg/animal. Two hours later the animals were euthanized by means of i.p. injection of Roumpun (Bayer) and Ketanest (Park-Davis). A blood sample from each animal was collected in a “vacutaner” tube (Becton Dickinson) and the plasma was separated according to the manufacturer's instructions. The TNF-α level in the plasma was determined by ELISA (Biosource, R&D Systems) according to the process prescribed by the manufacturer. The test sensitivity was <3 pg/mL TNF-α. The percentage inhibition of TNF-α production was calculated by the following equation:
% inhibition=[1−(TS−NC)/(PC−NC)]*100.

The compounds demonstrating a 30% or higher inhibition of TNF-α production at a dose of 10 mg/kg were considered active.

Writhing Test for Analgesic Activity

In this test, pain is induced with an injection of an irritant, usually acetic acid, into the peritoneal cavity of mice. The animals respond by the characteristic writhings, which gave the name of the test (Collier H. O. J. et al. Phamac. Chemother. 1968, 32, 295-310; Fukawa K. et al. J. Pharmacol. Meth., 1980, 4, 251-259; Schweizer A. et al. Agents Actions, 1988, 23, 29-31). This test is suitable for the determination of analgetic activity of the compounds of the invention.

Male BALB-/c mice (Charles River, Italy) at an age of 8 to 12 weeks were used. Methyl cellulose was administered p.o. to a control group, 30 minutes prior to i.p. administration of acetic acid in a concentration of 0.6%, whereas to the test groups a standard (acetyl salicylic acid) or test substances in methylcellulose were administered p.o. 30 minutes prior to i.p. administration of 0.6% acetic acid (volume 0.1 mL/10 g). Mice were individually placed under glass funnels and the number of writhings of each animal was recorded during a period of 20 minutes. The percentage inhibition of writhings was calculated according to the equation:
% inhibition=(mean value of number of writhings in the control group−number of writhings in the test group)/number of writhings in the control group×100.

The compounds demonstrating the same or better analgesic activity than acetyl salicylic acid were considered active.

In Vivo Model of LPS-Induced Shock in Mice

Male BALB/c mice at an age of 8 to 12 weeks (Charles River, Italy) were used. LPS isolated from Serratie marcessans (Sigma, L-6136) was diluted in sterile saline. The first LPS injection was administered intradermally in a dose of 4 μg/mouse. 18 to 24 hours later LPS was administered i.v. in a dose of 200 μg/mouse. To a control group, two LPS injections were administered in the above described manner. The test groups were administered the substances p.o. half an hour prior to each LPS administration. The survival after 24 hours was observed.

The compounds resulting in a 40% or better survival at a dose of 30 mg/kg were considered active.

Model of Lung Eosinophilia in Mice

Male Balb/C mice with a body weight of 20-25 g were randomly divided into groups, and sensitized by an i.p. injection of ovalbumin (OVA, Sigma) on day zero and day fourteen. On the twentieth day, the mice were subjected to a challenge test by i.n. (intranasal) application of OVA (positive control or test group) or PBS (negative control). 48 hours after i.n. application of OVA, the animals were anaesthetized and the lungs were rinsed with 1 mL of PBS. The cells were separated on a Cytospin 3 cytocentrifuge (Shandon). The cells were stained in Diff-Quick (Dade) and the percentage of eosinophils was determined by differential counting of at least 100 cells.

The compounds were administered daily i.n. or i.p. in different doses 2 days before the provocative test and up to the completion of the test. Compounds were administered as suspension either in carboxymethyl cellulose or in lactose solution.

Fluticasone and beclomethasone were used as standard anti-inflammatory substances for comparison.

Compounds 21 and 35 exhibit statistically significant inhibition of relative eosinophil number in BAL fluid when compared to the vehicle treated control group.

A compound is considered to be “active” if it is better than a positive control (i.e., Fluticasone or beclomethasone) in at least one inhibitory function (i.e., inhibition of eosinophil number) after stimulation with al least one stimulant.

Phorbol 12-myristate 13-acetate Induced Ear Oedema in CD1 Mice

Male CD1 mice (Iffa Credo, France) weighing 30-40 g were randomly grouped (n=8 in vehicle treated test group, dexamethasone treated control group as well as in groups treated with compounds to be assayed). Test compounds, dexamethasone, as well as vehicle (Trans-phase Delivery System, containing benzyl alcohol 10%, acetone 40%, and isopropanol 50%) (all from Kemika, Croatia), were administered topically to the internal surface of the left ear thirty minutes prior to administration of phorbol 12-myristate 13-acetate (PMA) (Sigma, USA). Test compounds were administered at a single dose of 500, 250 or 100 μg/15 μL/ear and dexamethasone at a single dose of 50 μg/15 μL/ear. Thirty minutes later, 0.01% PMA emulsion in acetone was applied topically to the same area of each animal in a volume of 12 μL/ear. During the treatment and challenge (stimulation), animals were anaesthetized by using inhalation anaesthesia. Six hours after the challenge, animals were euthanized by asphyxiation in 100% CO₂ atmosphere. For assessing the auricular oedema, 8 mm discs were cut out of left and right auricular pinna and weighed. The degree of oedema was calculated by subtracting the weight of 8 mm disc of the untreated ear from that of the treated contralateral ear.

Compound 14 statistically significantly inhibit PMA induced ear edema in CD1 mice in dose of 100 μg/ear.

A compound is considered to be “active” if it is better than a positive control (i.e., dexamethasone) in at least one inhibitory function (i.e., ear edema) after stimulation with al least one stimulant (e.g., PMA).

The compounds of Examples 1-51 demonstrate activity in at least two investigated tests. These results, however, only illustrate the biological activity of the compounds and do not limit the present invention in any way.

Preparation Processes with Examples

Intermediates

TABLE 1

| | |
|---|---|
| | Formula VII |
| 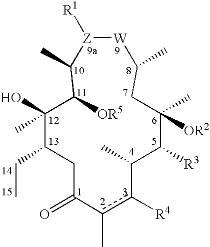 | |

| | | | | | | | | | | | C2-C3 |
|---|---|---|---|---|---|---|---|---|---|---|---|
| Com. | M | Z | W | R¹ | R² | R³ | R⁴ | R⁵ | R⁶ | R⁷ | bond |
| M_{L}1 | M1 | N | CH₂ | L1 | H | S¹ | S² | H | CH₃ | H | Single |
| M_{L}2 | M2 | N | CH₂ | L1 | H | S¹ | S² | H | H | H | Single |
| M_{L}3 | M3 | N | CH₂ | L1 | H | S¹ | S² | H | C(O) | H | Single |
| | | | | | | | | | CH₃ | | |
| M_{L}4 | M4 | N | CH₂ | L1 | H | S¹ | OH | H | CH₃ | / | Single |
| M_{L}5 | M5 | N | CH₂ | L1 | H | OH | OH | H | / | / | Single |
| M_{L}6 | M6 | N | CH₂ | CH₃ | H | S¹ | S² | H | L1 | H | Single |
| M_{L}7 | M7 | N | CH₂ | CH₃ | H | S¹ | S² | H | CH₃ | L2 | Single |
| M_{L}8 | M8 | N | CH₂ | CH₃ | H | S¹ | S² | L3 | CH₃ | H | Single |
| M_{L}9 | M9 | C | bond | ═O | CH₃ | S¹ | S² | H | L1 | H | Single |
| M_{L}10 | M10 | C | bond | L4 | H | S¹ | S² | H | CH₃ | H | Single |
| | M11 | N | CH₂ | L1 | H | OH | H | H | / | / | Double |

L1 = —(CH₂)₃—NH₂
L2 = —CH₂NH—(CH₂)₂—NH₂
L3 = —C(O)NH—(CH₂)₄—NH₂
L4 = ═N—O—(CH₂)₅—NH₂

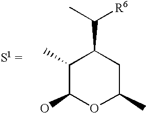

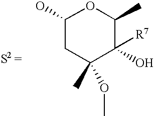

9a-Aza-9a-homoerythromycin amines M_{L}1 and M_{L}4 may be prepared according to procedures described in international patent application WO 02/055531 A1. Amine M_{L}5 may be prepared according to procedures described in international patent application WO 2004/09449 A1. Amines M_{L}2, M_{L}3 and M_{L}6-M_{L}10 may be prepared according to procedures described in international patent application WO 2004/005310 A2.

TABLE 2

| | |
|---|---|
| | Formula VIII |
| 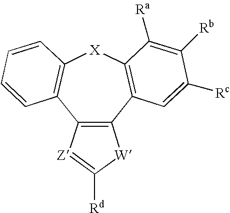 | |

| Inter. | X | Z′ | W′ | R^{a} | R^{b} | R^{c} | R^{d} |
|---|---|---|---|---|---|---|---|
| D1 | S | CH | S | H | H | H | CH₂OC(O)(CH₂)₂COOH |
| D2 | S | CH | S | H | H | H | CH═CH—COOH |
| D3 | S | CH | S | H | H | H | CHO |
| D4 | S | CH | S | H | H | H | COOH |
| D5 | S | CH | S | H | H | H | CH₂COOH |
| D6 | S | CH | S | H | H | H | CH₂CH₂COOH |
| D7 | S | CH | S | H | H | Cl | CH═CHCOOH |
| D8 | S | CH | S | H | Cl | H | CH═CHCOOH |
| D9 | S | CH | S | H | H | CH₃ | CH═CHCOOH |
| D10 | S | CH | S | H | H | F | CH═CHCOOH |
| D11 | S | CH | S | H | H | F | CH₂CH₂COOH |
| D12 | CH₂ | CH | S | H | H | H | CH═CHCOOH |
| D13 | O | CH | S | H | H | Cl | CHO |
| D14 | O | CH | S | H | H | Cl | COOH |
| D15 | NH | CH | S | H | H | H | COOH |
| D16 | N— | CH | S | H | H | H | COOH |
| | C(O)- | | | | | | |
| | Ph | | | | | | |
| D17 | NH | CH | S | H | H | H | CH═CHCOOH |
| D18 | O | N | O | H | H | Cl | CH₂CH₂COOH |
| D19 | O | N | O | H | H | Cl | CH₂Br |
| D20 | S | N | O | H | H | H | CH₂CH₂COOH |
| D21 | S | N | O | H | H | H | CH₂Br |
| D22 | S | CH | NH | H | H | H | CHO |
| D23 | S | CH | S | H | CH₃ | H | CHO |
| D24 | S | CH | S | H | CH₃ | H | CH═CHCOOH |

Intermediates D1-D17, D23 and D24 may be prepared according to procedures described in international patent application WO 01/87890 A1. Intermediates D18-D21 may be prepared according to procedures described in international patent application WO 03/084964 A1. Intermediate D22 may be prepared according to procedures described in international patent application WO 03/097648 A1.

### EXAMPLE 1

### Compound 1

### Formula I: M=M1, L=L1, D=D1

To a suspension of compound D1 (120 mg; approximately 0.18 mmol under 60% purity assumption) in dry CH₂Cl₂ (5 mL) under argon, triethylamine (0.228 mL; 1.64 mmol) was added resulting in a clear solution. Subsequently, 1-hydroxybenzotriazole (49 mg; 0.36 mmol), compound M_{L}1 (144 mg; 0.18 mmol) and 1-(3-dimethylaminopropyl)-3-ethyl-carbodiimide hydrochloride (139 mg; 0.73 mmol) were added. The reaction mixture was stirred for 4 hours at room temperature. The solvent was evaporated under reduced pressure and the residue purified on a silica gel column (eluant: CH₂Cl₂—MeOH—NH₄OH, 90:8:1). 107 mg of the compound 1 were obtained.

MS (m/z): 1170.79 [MH]⁺,

IR (KBr) cm⁻¹: 3435, 3058, 2971, 2936, 2876, 2831, 2786, 1736, 1656, 1546, 1459, 1421, 1377, 1327, 1252, 1165, 1109, 1053, 1012, 1000, 957, 896, 863, 836, 805, 760, 733, 701, 639.

### EXAMPLE 2

### Compound 2

### Formula I: M=M5, L=L1, D=D1

To a suspension of compound D1 (125 mg; approximately 0.22 mmol under 70% purity assumption) in dry CH₂Cl₂ (5 mL) under argon, triethylamine (0.277 mL; 1.99 mmol) was added resulting in a clear solution. Subsequently, 1-hydroxybenzotriazole (60 mg; 0.44 mmol), compound M_{L}5 (105 mg; 0.22 mmol) and 1-(3-dimethylaminopropyl)-3-ethyl-carbodiimide hydrochloride (169 mg; 0.88 mmol) were added. The reaction mixture was stirred for 4 hours at room temperature. The solvent was evaporated under reduced pressure and the residue purified on a silica gel column (eluant: CH₂Cl₂—MeOH—NH₄OH, 90:9:1.5). 90 mg of the compound 2 were obtained.

MS (m/z): 855.54 [MH]⁺,

IR (Br) cm⁻¹: 3415, 3060, 2971, 2934, 2875, 1720, 1655, 1546, 147, 1459, 1375, 1352, 1254, 1162, 1089, 1052, 1037, 1001, 973, 958, 899, 850, 809, 760, 733, 704, 670.

### EXAMPLE 3

### Compound 3

### Formula I: M=M6, L=L1, D=D2

To a suspension of compound D2 (80 mg; 0.24 mmol) in dry CH₂Cl₂ (5 mL) under argon, triethylamine (0.299 mL; 2.14 mmol) was added resulting in a clear solution. Subsequently, 1-hydroxybenzotriazole (64 mg; 0.48 mmol), compound M_{L}6 (189 mg; 0.24 mmol) and 1-(3-dimethylaminopropyl)-3-ethyl-carbodiimide hydrochloride (183 mg; 0.95 mmol) were added. The reaction mixture was stirred for 7 hours at room temperature. The solvent was evaporated under reduced pressure and the residue purified on a silica gel column (eluant: CHCl₃—MeOH—NH₄OH, 90:8:1). 136 mg of the compound 3 were obtained.

MS (m/z): 1110.26 [MH]⁺,

IR (KBr) cm⁻¹: 3433, 2969, 2934, 2876, 1729, 1664, 1618, 1560, 1544, 1528, 1459, 1379, 1327, 1256, 1177, 1105, 1082, 1055, 1012, 999, 960, 902, 865, 840, 795, 760, 732, 661.

### EXAMPLE 4

### Compound 4

### Formula I: M=M7, L=L2, D=D2

To a suspension of compound D2 (35 mg; 0.10 mmol) in dry CH₂Cl₂ (5 mL) under argon, triethylamine (0.140 mL; 1 mmol) was added resulting in a clear solution. Subsequently, 1-hydroxybenzotriazole (27 mg; 0.2 mmol), compound M_{L}7 (75 mg; 0.10 mmol) and 1-(3-dimethylaminopropyl)-3-ethyl-carbodiimide hydrochloride (76.40 mg; 0.40 mmol) were added. The reaction mixture was stirred at room temperature overnight. The solvent was evaporated under reduced pressure and crude product was purified on a silica gel column (eluant: CHCl₃—MeOH—NH₄OH, 90:9:1.5). 43 mg of the compound 4 was obtained.

MS (m/z): 1139.39 [MH]⁺,

IR (KBr) cm⁻¹: 3424, 3060, 2970, 2936, 2876, 2831, 1728, 1656, 1618, 1560, 1545, 1457, 1421, 1376, 1327, 1268, 1178, 1109, 1051, 995, 972, 896, 838, 795, 759, 732, 640.

### EXAMPLE 5

### Compound 5

### Formula I: M=M1, L=L1, D=D2

To a suspension of compound D2 (80 mg; 0.24 mmol) in dry CH₂Cl₂ (5 mL) under argon, triethylamine (0.299 mL; 2.14 mmol) was added resulting in a clear solution. Subsequently, 1-hydroxybenzotriazole (64 mg; 0.48 mmol), compound M_{L}1 (189 mg; 0.24 mmol) and 1-(3-dimethylaminopropyl)-3-ethyl-carbodiimide hydrochloride (183 mg; 0.95 mmol) were added. The reaction mixture was stirred for 5 hours at room temperature. The solvent was evaporated under reduced pressure and the residue purified on a silica gel column (eluant: CH₂Cl₂—MeOH—NH₄OH, 90:9:1.5). 173 mg of the compound 5 were obtained.

MS (m/z): 1110.79 [MH]⁺,

IR (KBr) cm⁻¹: 3440, 3059, 2971, 2936, 2876, 2831, 2786, 1728, 1659, 1615, 1548, 1531, 1455, 1377, 1327, 1267, 1181, 1166, 1109, 1053, 1012, 960, 896, 863, 837, 805, 759, 732, 700, 674, 640.

### EXAMPLE 6

### Compound 6

### Formula I: M=M5, L=L1, D=D2

To a suspension of compound D2 (80 mg; 0.24 mmol) in dry CH₂Cl₂ (5 mL) under argon, triethylamine (0.299 mL; 2.14 mmol) was added resulting in a clear solution. Subsequently, 1-hydroxybenzotriazole (64 mg; 0.48 mmol), compound M_{L}5 (113 mg; 0.24 mmol) and 1-(3-dimethylaminopropyl)-3-ethyl-carbodiimide hydrochloride (183 mg; 0.95 mmol) were added. The reaction mixture was stirred for 6 hours at room temperature. The solvent was evaporated under reduced pressure and the residue purified on a silica gel column (eluant: CH₂Cl₂—MeOH—NH₄OH, 90:8:1). 121 mg of the compound 6 were obtained.

MS (m/z): 795.56 [MH]⁺,

IR (KBr) cm⁻¹: 3376, 3061, 2972, 2934, 2875, 1712, 1651, 1614, 1549, 1455, 1415, 1374, 1350, 1330, 1267, 1253, 1181, 1137, 1089, 1052, 958, 896, 839, 810, 757, 665.

### EXAMPLE 7

### Compound 7

### Formula I: M=M10, L=L4, D=D2

To a suspension of compound D2 (60 mg; 0.18 mmol) in dry CH₂Cl₂ (5 mL) under argon, triethylamine (0.233 mL; 1.67 mmol) was added resulting in a clear solution. Subsequently, 1-hydroxybenzotriazole (48 mg; 0.48 mmol), compound M_{L}10 (149 mg; 0.18 mmol) and 1-(3-dimethylaminopropyl)-3-ethyl-carbodiimide hydrochloride (137 mg; 0.71 mmol) were added. The reaction mixture was stirred for 6 hours at room temperature. The solvent was evaporated under reduced pressure and the residue purified on a silica gel column (eluant: CH₂Cl₂—MeOH—NH₄OH, 90:8:1). 139 mg of the compound 7 were obtained.

MS (m/z): 1153.00 [MH]⁺,

IR (KBr) cm⁻¹: 3444, 3057, 2972, 2936, 2875, 2831, 2786, 1736, 1660, 1620, 1547, 1456, 1403, 1378, 1344, 1329, 1268, 1167, 1110, 1085, 1053, 1012, 957, 894, 864, 836, 804, 759, 732, 698, 638.

### EXAMPLE 8

### Compound 8

### Formula I: M=M9, L=L1, D=D2

To a suspension of compound D2 (80 mg; 0.24 mmol) in dry CH₂Cl₂ (5 mL) under argon, triethylamine (0.298 mL; 2.14 mmol) was added resulting in a clear solution. Subsequently, 1-hydroxybenzotriazole (64 mg; 0.48 mmol), compound M_{L}9 (188 mg; 0.24 mmol) and 1-(3-dimethylaminopropyl)-3-ethyl-carbodiimide hydrochloride (182 mg; 0.95 mmol) were added. The reaction mixture was stirred for 6 hours at room temperature. The solvent was evaporated under reduced pressure and the residue purified on a silica gel column (eluant: CH₂Cl₂—MeOH—NH₄OH, 90:8:1). 82 mg of the compound 8 were obtained.

MS (m/z): 1109.65 [MH]⁺,

IR (KBr) cm⁻¹: 3444, 3055, 2972, 2937, 2879, 2832, 1732, 1688, 1660, 1616, 1526, 1456, 1404, 1378, 1346, 1330, 1285, 1267, 1246, 1169, 1109, 1054, 1010, 969, 961, 935, 902, 891, 864, 839, 803, 757, 697, 665, 639.

### EXAMPLE 9

### Compound 9

### Formula I: M=M1, L=L1, D=D3

Compound D3 (100 mg; 0.34 mmol) was dissolved in MeOH (15 mL). Compound M_{L}1 (269 mg; 0.34 mmol), NaBH₃CN (21.3 mg; 0.34 mmol) and drop of acetic acid were added. The reaction mixture was stirred at room temperature overnight. The reaction mixture volume was reduced by evaporation under reduced pressure and the residue was extracted between EtOAc and 50% solution of NaHCO₃. The organic phase was washed with H₂O twice and once with brine, dried over Na₂SO₄ and evaporated. The residue was purified on a silica gel column (eluant: CHCl₃—MeOH—NH₄OH, 6:1:0.1). 13 mg of the compound 9 were obtained.

MS (m/z): 1070.34 □MH□⁺.

### EXAMPLES 10 AND 11

### Compound 10

### Formula I: M=M1, L=L1, D=D4

### Compound 11

### Formula I: M=M1, L=L1, D=D5

To a suspension of mixture of compounds D4 and D5 (36 mg; with approximate ratio 31/50 in the mixture according to HPLC-MS) in dry CH₂Cl₂ (3 mL) under argon, triethylamine (0.138 mL; 0.99 mmol) was added resulting in a clear solution. Subsequently, 1-hydroxybeizotriazole (30 mg; 0.22 mmol), compound M_{L}1 (87 mg; 0.11 mmol) and 1-(3-dimethylaminopropyl)-3-ethyl-carbodiimide hydrochloride (84 mg; 0.44 mmol) were added. The reaction mixture was stirred at room temperature overnight. The solvent was evaporated under reduced pressure and the residue purified three times on a silica gel columns (eluants: CHCl₃—MeOH—NH₄OH, 90:9:1.5; CH₂Cl₂—MeOH—NH₄OH, 90:8:1 and EtOAc-Et₃N, 96:4). 22 mg of the compound 10 and 31 mg of the compound 11 were obtained.

Compound 10: MS (m/z): 1084.26 [MH]⁺,

IR (KBr) cm⁻¹: 3448, 3059, 2971, 2935, 1729, 1655, 1638, 1551, 1524, 1458, 1376, 1284, 1167, 1109, 1053, 1012, 958, 896, 834, 804, 759, 732, 695, 670.

Compound 11: MS (m/z): 1098.21 [MH]⁺,

IR (KBr) cm⁻¹: 3424, 3058, 2971, 2935, 1730, 1655, 1560, 1545, 1476, 1459, 1376, 1251, 1167, 1109, 1053, 1012, 957, 897, 835, 805, 759, 732, 641.

### EXAMPLE 12

### Compound 12

### Formula I: M=M1, L=L1, D=D6

### Compound 5

### Formula I: M=M1, L=L1, D=D2

To a suspension of mixture of compounds D2 and D6 (171 mg; with approximate ratio 51/46 in the mixture according to HPLC-MS) in dry CH₂Cl₂ (10 mL) under argon, triethylamine (0.633 mL; 4.54 mmol) was added resulting in a clear solution. Subsequently, 1-hydroxybenzotriazole (136 mg; 1.01 mmol), compound M_{L}1 (399 mg; 0.50 mmol) and 1-(3-dimethylaminopropyl)-3-ethyl-carbodiimide hydrochloride (387 mg; 2.02 mmol) were added. The reaction mixture was stirred at room temperature overnight. The solvent was evaporated under reduced pressure and the residue purified twice on a silica gel columns (eluants: CH₂Cl₂—MeOH—NH₄OH, 90:9:1.5; then EtOAc-Et₃N, 96:4). 18 mg of the pure compound 12 and 43 mg of the compound 5 were obtained.

Compound 12: MS (m/z): 1112.29 [MH]⁺.

### EXAMPLE 13

### Compound 13

### Formula I: M=M1, L=L1, D=D7

To a suspension of compound D7 (100 mg; 0.27 mmol) in dry CH₂Cl₂ (5 mL) under argon, triethylamine (0.338 mL; 2.42 mmol) was added resulting in a clear solution. Subsequently, 1-hydroxybenzotriazole (73 mg; 0.54 mmol), compound M_{L}1 (213 mg; 0.27 mmol) and 1-(3-dimethylaminopropyl)-3-ethyl-carbodiimide hydrochloride (207 mg; 1.08 mmol) were added. The reaction mixture was stirred for 6 hours at room temperature. The solvent was evaporated under reduced pressure and the residue purified on a silica gel column (eluant: CH₂Cl₂—MeOH—NH₄OH, 90:9:1.5). 186 mg of the compound 13 were obtained.

MS (m/z): 1144.62 [MH]⁺,

IR (KBr) cm⁻: 3427, 3060, 2971, 2936, 2875, 2831, 2786, 1727, 1659, 1619, 1549, 1531, 1455, 1377, 1327, 1267, 1248, 1166, 1095, 1053, 1012, 960, 896, 866, 837, 817, 796, 757, 665, 639.

### EXAMPLE 14

### Compound 14

### Formula I: M=M9, L=L1, D=D7

To a suspension of compound D7 (100 mg; 0.27 mmol) in dry CH₂Cl₂ (5 mL) under argon, triethylamine (0.338 mL; 2.42 mmol) was added resulting in a clear solution. Subsequently, 1-hydroxybenzotriazole (73 mg; 0.48 mmol), compound M_{L}9 (213 mg; 0.27 mmol) and 1-(3-dimethylaminopropyl)-3-ethyl-carbodiimide hydrochloride (207 mg; 1.08 mmol) were added. The reaction mixture was stirred for 6 hours at room temperature. The solvent was evaporated under reduced pressure and the residue purified on a silica gel column (eluant: CHC3—MeOH—NH₄OH, 90:8:1). 36 mg of the pure compound 14 were obtained.

MS (m/z): 1143.60 [MH]⁺,

IR (KBr) cm⁻¹: 3449, 3057, 2971, 2936, 2880, 2832, 1733, 1688, 1659, 1618, 1575, 1546, 1527, 1461, 1402, 1377, 1346, 1330, 1285, 1266, 1247, 1169, 1108, 1053, 1009, 963, 937, 902, 890, 865, 838, 818, 796, 758, 724, 696, 663, 635.

### EXAMPLE 15

### Compound 15

### Formula I: M=M6, L=L1, D=D7

To a suspension of compound D7 (80 mg; 0.21 mmol) in dry CH₂Cl₂ (5 mL) under argon, triethylamine (0.271 mL; 1.94 mmol) was added resulting in a clear solution. Subsequently, 1-hydroxybenzotriazole (58 mg; 0.43 mmol), compound M_{L}6 (171 mg; 0.21 mmol) and 1-(3-dimethylaminopropyl)-3-ethyl-carbodiimide hydrochloride (165 mg; 0.86 mmol) were added. The reaction mixture was stirred for 6 hours at room temperature. The solvent was evaporated under reduced pressure and the residue purified on a silica gel column (eluant: CH₂Cl₂—MeOH—NH₄OH, 90:8:1). 103 mg of the compound 13 were obtained.

MS (m/z): 1144.38 [MH]⁺,

IR (KBr) cm⁻¹: 3432, 2970, 2935, 2875, 1726, 1655, 1618, 1577, 1547, 1524, 1459, 1377, 1324, 1267, 1179, 1167, 1107, 1054, 1012, 999, 961, 899, 866, 841, 818, 795, 759, 665.

### EXAMPLE 16

### Compound 16

### Formula I: M=M7, L=L2, D=D7

To a suspension of compound D7 (37 mg; 0.10 mmol) in dry CH₂Cl₂ (5 mL) under argon, triethylamine (0.140 mL; 1 mmol) was added resulting in a clear solution. Subsequently, 1-hydroxybenzotriazole (27 mg; 0.2 mmol), compound M_{L}7 (75 mg; 0.10 mmol) and 1-(3-dimethylaminopropyl)-3-ethyl-carbodiimide hydrochloride (76.40 mg; 0.40 mmol) were added. The reaction mixture was stirred at room temperature overnight. The solvent was evaporated under reduced pressure and crude product was purified on a silica gel column (eluant: CH₂Cl₂—MeOH—NH₄OH, 90:9:1.5). 38 mg of the compound 16 were obtained.

MS (m/z): 1173.29 [MH]⁺,

IR (KBr) cm⁻¹: 3430, 3057, 2970, 2935, 2870, 1729, 1665, 1619, 1560, 1550, 1458, 1380, 1329, 1259, 1177, 1108, 1075, 1051, 1012, 993, 970, 895, 867, 838, 816, 795, 763, 742, 663, 641.

### EXAMPLE 17

### Compound 17

### Formula I: M=M1, L=L1, D=D8

To a suspension of compound D8 (200 mg; 0.54 mmol) in dry CH₂Cl₂ (5 mL) under argon, triethylamine (0.588 mL; 4.22 mmol) was added resulting in a clear solution. Subsequently, 1-hydroxybenzotriazole (146.6 mg; 1.08 mmol), compound M_{L}1 (427.6 mg; 0.54 mmol) and 1-(3-dimethylaminopropyl)-3-ethyl-carbodiimide hydrochloride (373.2 mg; 1.95 mmol) were added. The reaction mixture was stirred at room temperature overnight. The solvent was evaporated under reduced pressure and crude product was purified on a silica gel column (eluant: CH₂Cl₂—MeOH—NH₄OH, 90:8:1). 270 mg of the compound 17 were obtained.

MS (m/z): 1145.13 [MH]⁺.

### EXAMPLE 18

### Compound 18

### Formula I: M=M4, L=L1, D=D8

To a solution of water (10.0 mL) and conc. HCl (1.0 mL), compound 17 (100 mg; 0,09 mmol) was added. The reaction mixture was stirred at room temperature for 2 hours. The reaction was saturated with sodium chloride and was adjusted to pH 8 with aqueous ammonium hydroxide. The solution was extracted with EtOAc (3×10 mL), and the extracts were dried over anhydrous K₂CO₃ and evaporated. 54 mg of the compound 18 were obtained.

MS (m/z): 986.51 [MH]⁺,

IR (KBr) cm⁻¹: 3444, 3062, 2972, 2936, 2876, 1709, 1655, 1618, 1560, 1545, 1476, 1457, 1375, 1327, 1265, 1174, 1100, 1074, 1050, 958, 899, 863, 838, 820, 802, 762, 728, 633.

### EXAMPLE 19

### Compound 19

### Formula I: M=M5, L=L1, D=D8

To a suspension of compound D8 (100 mg; 0.27 mmol) in dry CH₂Cl₂ (5 mL) under argon, triethylamine (0.294 mL; 2.11 mmol) was added resulting in a clear solution. Subsequently, 1-hydroxybenzotriazole (73.2 mg; 0.54 mmol), compound M_{L}5 (128.8 mg; 0.27 mmol) and 1-(3-dimethylaminopropyl)-3-ethyl-carbodiimide hydrochloride (186.5 mg; 0.97 mmol) were added. The reaction mixture was stirred at room temperature overnight. The solvent was evaporated under reduced pressure and crude product was purified on a silica gel column (eluant: CH₂Cl₂—MeOH—NH₄OH, 90:8:1). 57 mg of the compound 19 were obtained.

MS (m/z): 829.3 [MH]⁺,

IR (KBr) cm⁻¹: 3432, 2970, 2932, 2875, 1710, 1656, 1616, 1562, 1545, 1476, 1458, 1424, 1374, 1266, 1248, 1181, 1099, 1052, 960, 839, 819, 802, 762.

### EXAMPLE 20

### Compound 20

### Formula I: M=M1, L=L1, D=D9

To a suspension of compound D9 (200 mg; 0.57 mmol) in dry CH₂Cl₂ (10 mL) under argon, triethylamine (0.622 mL; 4.46 mmol) was added resulting in a clear solution. Subsequently, 1-hydroxybenzotriazole (155 mg; 1.15 mmol), compound M_{L}1 (452 mg; 0.57 mmol) and 1-(3-dimethylaminopropyl)-3-ethyl-carbodiimide hydrochloride (395 mg; 2.06 mmol) were added. The reaction mixture was stirred at room temperature overnight. The solvent was evaporated under reduced pressure and crude product was purified on a silica gel column two times (eluant: CH₂Cl₂—MeOH—NH₄OH, 90:8:1). 170 mg of the compound 20 were obtained.

MS (m/z): 1125.2 [MH]⁺,

IR (KBr) cm⁻¹: 3434, 2971, 2936, 2881, 1722, 1657, 1619, 1526, 1459, 1377, 1327, 1267, 1167, 1110, 1053, 1012, 960, 898, 837, 815, 762, 730.

### EXAMPLE 21

### Compound 21

### Formula I: M=M4, L=L1, D=D9

To a solution of water (8.0 mL) and conc. HCl (0.8 mL), compound 20 (80 mg; 0,07 mmol) was added. The reaction mixture was stirred at room temperature for 2 hours. The reaction was saturated with sodium chloride and was adjusted to pH 8 with aqueous ammonium hydroxide. The solution was extracted with EtOAc (3×10 mL), and the extracts were dried over anhydrous K₂CO₃ and evaporated. 61 mg of the compound 21 were obtained.

MS (m/z): 966.53 [MH]⁺,

IR (KBr) cm⁻¹: 3424, 2970, 2936, 2875, 2731, 2619, 1697, 1655, 1630, 1561, 1535, 1509, 1450, 1400, 1374, 1265, 1175, 1111, 1074, 1050, 1008, 978, 846, 833, 762, 702, 669.

### EXAMPLE 22

### Compound 22

### Formula I: M=M5, L=L1, D=D9

To a suspension of compound D9 (100 mg; 0.29 mmol) in dry CH₂Cl₂ (5 mL) under argon, triethylamine (0.310 mL; 2.23 mmol) was added resulting in a clear solution. Subsequently, 1-hydroxybenzotriazole (77.4 mg; 0.57 mmol), compound M_{L}5 (136.1 mg; 0.29 mmol) and 1-(3-dimethylaminopropyl)-3-ethyl-carbodiimide hydrochloride (197.2 mg; 1.04 mmol) were added. The reaction mixture was stirred at room temperature overnight. The solvent was evaporated under reduced pressure and crude product was purified twice on a silica gel column (eluant: CH₂Cl₂—MeOH—NH₄OH, 90:8:1). 89 mg of the compound 22 were obtained.

MS (m/z): 809.35 [MH]⁺,

IR (KBr) cm⁻¹: 3424, 2972, 2934, 2875, 1710, 1656, 1614, 1545, 1458, 1374, 1351, 1267, 1180, 1140, 1090, 1052, 960, 839, 816, 760, 665.

### EXAMPLE 23

### Compound 23

### Formula I: M=M1, L=L1, D=D10

To a suspension of compound D10 (100 mg; 0.29 mmol) in dry CH₂Cl₂ (5 mL) under argon, triethylamine (0.307 mL; 2.23 mmol) was added resulting in a clear solution. Subsequently, 1-hydroxybenzotriazole (76.5 mg; 0.56 mmol), compound M_{L}1 (223.4 mg; 0.29 mmol) and 1-(3-dimethylaminopropyl)-3-ethyl-carbodiimide hydrochloride (195 mg; 1.02 mmol) were added. The reaction mixture was stirred at room temperature overnight. The solvent was evaporated under reduced pressure and crude product was purified twice on a silica gel column (eluant: CH₂Cl₂—MeOH—NH₄OH, 90:9:1.5). 150 mg of the compound 23 were obtained.

MS (m/z): 1128.43 [MH]⁺,

IR (KBr) cm⁻¹: 3442, 3063, 3972, 2937, 1875, 2831, 2787, 1726, 1656, 1618, 1597, 1572, 1535, 1459, 1377, 1327, 1268, 1250, 1171, 1109, 1053, 1003, 960, 897, 866, 837, 816, 760, 666, 640.

### EXAMPLE 24

### Compound 24

### Formula I: M=M6, L=L1, D=D10

To a suspension of compound D10 (100 mg; 0.29 mmol) in dry CH₂Cl₂ (5 mL) under argon, triethylamine (0.307 mL; 2.23 mmol) was added resulting in a clear solution. Subsequently, 1-hydroxybenzotriazole (76.5 mg; 0.56 mmol), compound M_{L}1 (223.4 mg; 0.29 mmol) and 1-(3-dimethylaminopropyl)-3-ethyl-carbodiimide hydrochloride (195 mg; 1.02 mmol) were added. The reaction mixture was stirred at room temperature overnight. The solvent was evaporated under reduced pressure and crude product was purified on a silica gel column (eluant: CH₂Cl₂—MeOH—NH₄OH, 90:9:1.5). 180 mg of the compound 24 were obtained.

MS (m/z): 1128.3 [MH]⁺,

IR (KBr) cm⁻¹: 3438, 3062, 2970, 2936, 2875, 1726, 1657, 1618, 1597, 1572, 1528, 1460, 1377, 1326, 1269, 1252, 1171, 1107, 1055, 1002, 960, 900, 866, 840, 814, 763, 726, 640.

### EXAMPLE 25

### Compound 25

### Formula I: M=M1, L=L1, D=D11

### Compound 23

### Formula I: M=M1, L=L1, D=D10

To a suspension of mixture of compounds D10 and D11 (30 mg; (with approximate ratio 50/50 in the mixture according to HPLC-MS) in dry CH₂Cl₂ (5 mL) under argon, triethylamine (0.092 mL; 0.66 mmol) was added resulting in a clear solution. Subsequently, 1-hydroxybenzotriazole (22.8 mg; 0.17 mmol), compound M_{L}1 (66.6 mg; 0.08 mmol) and 1-(3-dimethylaminopropyl)-3-ethyl-carbodiimide hydrochloride (58.1 mg; 0.30 mmol) were added. The reaction mixture was stirred at room temperature overnight. The solvent was evaporated under reduced pressure and crude product was purified on a silica gel column (eluant: CH₂Cl₂—MeOH—NH₄OH, 90:9:1.5). 30 mg of the mixture of the compounds 25 and 23 (with approximate ratio 50/50 in the mixture according to HPLC-MS) were obtained.

Compound 25: MS (m/z): 1130.2 [MH]⁺.

### EXAMPLE 26

### Compound 26

### Formula I: M=M1, L=L1, D=D12

To a suspension of compound D12 (80 mg; 0.25 mmol) in dry CH₂Cl₂ (5 mL) under argon, triethylamine (0.315 mL; 2.26 mmol) was added resulting in a clear solution. Subsequently, 1-hydroxybenzotriazole (68 mg; 0.50 mmol), compound M_{L}1 (199 mg; 0.25 mmol) and 1-(3-dimethylaminopropyl)-3-ethyl-carbodiimide hydrochloride (193 mg; 1.00 mmol) were added. The reaction mixture was stirred for 6 hours at room temperature. The solvent was evaporated under reduced pressure and the residue purified on a silica gel column (eluant: CHCl₃—MeOH—NH₄OH, 90:8:1). 207 mg of the compound 26 were obtained.

MS (m/z): 1092.49 [MH]⁺,

IR (KBr) cm⁻¹: 3433, 2972, 2936, 2877, 2831, 2787, 2831, 2787, 1727, 1655, 1617, 1544, 1493, 1458, 1377, 1328, 1278, 1259, 1167, 1110, 1092, 1053, 1012, 960, 897, 863, 838, 806, 757, 690, 665, 618.

### EXAMPLE 27

### Compound 27

### Formula I: M=M6, L=L1, D=D12

To a suspension of compound D12 (80 mg; 0.25 mmol) in dry CH₂Cl₂ (5 mL) under argon, triethylamine (0.315 mL; 2.26 mmol) was added resulting in a clear solution. Subsequently, 1-hydroxybenzotriazole (68 mg; 0.50 mmol), compound M_{L}6 (199 mg; 0.25 mmol) and 1-(3-dimethylaminopropyl)-3-ethyl-carbodiimide hydrochloride (193 mg; 1.00 mmol) were added. The reaction mixture was stirred for 16 hours at room temperature. The solvent was evaporated under reduced pressure and the residue purified on a silica gel column (eluant: CHCl₃—MeOH—NH₄OH, 90:8:1). 139 mg of the compound 27 were obtained.

MS (m/z): 1092.32 [MH]⁺,

IR (KBr) cm−1: 3423, 3062, 2970, 2936, 2877, 1726, 1655, 1617, 1542, 1523, 1493, 1460, 1377, 1326, 1258, 1177, 1109, 1054, 1012, 999, 960, 900, 861, 840, 795, 757, 691, 665, 644, 618.

### EXAMPLE 28

### Compound 28

### Formula I: M=M7, L=L2, D=D12

To a suspension of compound D12 (60 mg; 0.19 mmol) in dry CH₂Cl₂ (5 mL) under argon, triethylamine (0.236 mL; 1.69 mmol) was added resulting in a clear solution. Subsequently, 1-hydroxybenzotriazole (51 mg; 0.38 mmol), compound M_{L}7 (155 mg; 0.19 mmol) and 1-(3-dimethylaminopropyl)-3-ethyl-carbodiimide hydrochloride (144 mg; 0.75 mmol) were added. The reaction mixture was stirred for 16 hours at room temperature. The solvent was evaporated under reduced pressure and the residue purified twice on a silica gel columns (eluants: CH₂Cl₂—MeOH—NH₄OH, 90:9:1.5; then CHCl₃—MeOH—NH₄OH, 90:9:1.5). 34 mg of the pure compound 28 were obtained.

MS (m/z): 1121.23 [MH]⁺,

IR (KBr) cm⁻¹: 3428, 3062, 2937, 2831, 1728, 1660, 1616, 1519, 1494, 1456, 1379, 1328, 1276, 1258, 1176, 1110, 1049, 995, 896, 838, 795, 757, 666, 618.

### EXAMPLE 29

### Compound 29

### Formula I: M=M1, L=L1, D=D13

Compound D13 (200 mg; 0.64 mmol) was dissolved in MeOH (10 mL). Compound M_{L}1 (507 mg; 0.64 mmol), NaBH₃CN (40 mg; 0.64 mmol) and drop of acetic acid were added. The reaction mixture was stirred at room temperature overnight. The reaction mixture volume was reduced by evaporation under reduced pressure and the residue was extracted between EtOAc (10 mL) and 50% solution of NaHCO₃ (10 mL). The organic phase was washed with H₂O (2×10 mL) and with brine (10 mL), dried over Na₂SO₄ and evaporated. The residue was purified on a silica gel column (eluant: CH₂Cl₂—MeOH—NH₄OH, 90:8:1). 38 mg of the compound 29 were obtained.

MS (m/z): 1088.6 [MH]⁺,

IR (KBr) cm⁻¹: 3448, 2972, 2936, 2876, 2824, 2169, 1719, 1655, 1638, 1561, 1542, 1459, 1380, 1256, 1167, 1118, 1053, 1013, 957, 896, 833, 805, 772, 753, 727.

### EXAMPLE 30

### Compound 30

### Formula I: M=M1, L=L1, D=D14

To a suspension of compound D14 (100 mg; 0.31 mmol) in dry CH₂Cl₂ (5 mL) under argon, triethylamine (0.332 mL; 2.38 mmol) was added resulting in a clear solution. Subsequently, 1-hydroxybenzotriazole (82.7 mg; 0.61 mmol), compound M_{L}1 (241.3 mg; 0.31 mmol) and 1-(3-dimethylaminopropyl)-3-ethyl-carbodiimide hydrochloride (210.3 mg; 1.10 mmol) were added. The reaction mixture was stirred at room temperature overnight. The solvent was evaporated under reduced pressure and crude product was purified on a silica gel column (eluant: CH₂Cl₂—MeOH—NH₄OH, 90:8:1). 35 mg of the compound 30 were obtained.

MS (m/z): 1102.70 [MH]⁺,

IR (KBr) cm⁻¹: 3435, 2972, 2937, 2877, 1721, 1639, 1552, 1526, 1483, 1460, 1440, 1380, 1256, 1209, 1167, 1111, 1092, 1053, 1013, 958, 897, 873, 833, 807, 751.

### EXAMPLE 31

### Compound 31

### Formula I: M=M4, L=L1, D=D14

To a solution of water (10.0 mL) and conc. HCl (1.0 mL), compound 30 (100 mg; 0,09 mmol) was added. The reaction mixture was stirred at room temperature for 2 hours. The reaction was saturated with sodium chloride and was adjusted to pH 8 with aqueous ammonium hydroxide. The solution was extracted with EtOAc (3×10 mL), and the extracts were dried over anhydrous K₂CO₃ and evaporated. 81 mg of the compound 21 were obtained.

MS (m/z): 944.56 [MH]⁺,

IR (KBr) cm⁻¹: 3438, 2973, 2937, 2877, 1709, 1638, 1560, 1554, 1529, 1483, 1459, 1439, 1383, 1303, 1257, 1210, 1171, 1112, 1074, 1051, 993, 978, 957, 873, 833, 809, 772, 747, 653, 627.

### EXAMPLE 32

### Compound 32

### Formula I: M=M8, L=L3, D=D14

To a suspension of compound D14 (27 mg; 0.08 mmol) in dry CH₂Cl₂ (10 mL) under argon, triethylamine (0.101 mL; 0.72 mmol) was added resulting in a clear solution. Subsequently, 1-hydroxybenzotriazole (22 mg; 0.16 mmol), compound M_{L}8 (69 mg; 0.08 mmol) and 1-(3-dimethylaminopropyl)-3-ethyl-carbodiimide hydrochloride (62 mg; 0.32 mmol) were added. The reaction mixture was stirred at room temperature overnight. The solvent was evaporated under reduced pressure and crude product was purified on a silica gel column (eluant: CH₂Cl₂—MeOH—NH₄OH, 90:9:1.5). 16 mg of the compound 32 were obtained.

MS (m/z): 1174.19 [MH]⁺,

IR (KBr) cm⁻¹: 3449, 3061, 2972, 2937, 2877, 2831, 2789, 1730, 1649, 1552, 1528, 1483, 1460, 1379, 1257, 1167, 1110, 1053, 1013, 958, 898, 871, 833, 806, 771, 733, 700.

### EXAMPLE 33

### Compound 33

### Formula I: M=M5, L=L1, D=D14

To a suspension of compound D14 (200 mg; 0.61 mmol) in dry CH₂Cl₂ (10 mL) under argon, triethylamine (0.664 mL; 4.76 mmol) was added resulting in a clear solution. Subsequently, 1-hydroxybenzotriazole (165.4 mg; 1.22 mmol), compound M_{L}5 (290.6 mg; 0.61 mmol) and 1-(3-dimethylaminopropyl)-3-ethyl-carbodiimide hydrochloride (421 mg; 2.20 mmol) were added. The reaction mixture was stirred at room temperature overnight. The solvent was evaporated under reduced pressure and crude product was purified on a silica gel column (eluant: CHCl₃—MeOH—NH₄OH, 90:8:1). 150 mg of the compound 33 were obtained.

MS (m/z): 787.66 [MH]⁺,

IR (KBr) cm⁻¹: 3426, 2972, 2935, 2876, 1712, 1634, 1556, 1530, 1483, 1463, 1439, 1383, 1300, 1271, 1256, 1210, 1180, 1136, 1091, 1052, 992, 957, 895, 873, 832, 807, 771, 747, 724, 653, 626.

### EXAMPLE 34

### Compound 34

### Formula I: M=M6, L=L1, D=D14

To a suspension of compound D14 (83 mg; 0.25 mmol) in dry CH₂Cl₂ (5 mL) under argon, triethylamine (0.275 mL; 1.97 mmol) was added resulting in a clear solution. Subsequently, 1-hydroxybenzotriazole (68.6 mg; 0.51 mmol), compound M_{L}6 (200 mg; 0.25 mmol) and 1-(3-dimethylaminopropyl)-3-ethyl-carbodiimide hydrochloride (174.5 mg; 0.91 mmol) were added. The reaction mixture was stirred at room temperature overnight. The solvent was evaporated under reduced pressure and crude product was purified on a silica gel column (eluant: CH₂Cl₂—MeOH—NH₄OH, 90:9:1.5). 186 mg of the compound 34 were obtained.

MS (m/z): 1102.40 [MH]⁺,

IR (KBr) cm⁻¹: 3433, 3082, 2971, 2935, 2875, 1720, 1638, 1578, 1560, 1554, 1528, 1483, 1460, 1439, 1382, 1272, 1256, 1209, 1167, 1109, 1055, 997, 958, 901, 873, 833, 807, 770, 750, 702, 654, 627.

### EXAMPLE 35

### Compound 35

### Formula I: M=M2, L=L1, D=D14

Compound 30 (300 mg; 0.27 mmol) was dissolved in MeOH (20 mL) and treated with NaOAcx3H₂O (185 mg; 48.92 mmol) and 12 (73.3 mg; 2.89 mmol). The solution was irradiated with a 500 W halogen lamp and stirred at ambient temperature. After 2 hours TLC indicated complete conversion of the starting compound to a new, more polar material. The excess of 12 was quenched by the dropwise addition of 1 M Na₂S₂O₃. The solvent was evaporated under reduced pressure and crude product was purified twice on a silica gel column (eluant: CH₂Cl₂—MeOH—NH₄OH, 90:8:1). 50 mg of the compound 35 were obtained.

MS (m/z): 1088.3 [MH]⁺.

### EXAMPLES 36 AND 39

### Compound 36

### Formula I: M=M1, L=L1, D=D15

Compound 39: (Formula I: M=M1, L=L1, D=D16

To a suspension of mixture of compounds D15 and D16 (100 mg; with approximate ratio 53/22 in the mixture according to HPLC-MS) in dry CH₂Cl₂ (10 mL) under argon, triethylamine (0.380 mL; 2.73 mmol) was added resulting in a clear solution. Subsequently, 1-hydroxybenzotriazole (80 mg; 0.6 mmol), compound M_{L}1 (230 mg; 0.3 mmol) and 1-(3-dimethylaminopropyl)-3-ethyl-carbodiimide hydrochloride (230 mg; 1.20 mmol) were added. The reaction mixture was stirred at room temperature overnight. The solvent was evaporated under reduced pressure and crude product was purified on a silica gel column (eluant: CH₂Cl₂—MeOH—NH₄OH, 90:8:1). 29 mg of the compound 36 and 13 mg of the compound 39 were obtained.

Compound 36: MS (m/z): 1167.35 [MH]⁺,

IR (KBr) cm⁻¹: 3448, 3059, 2971, 2935, 2875, 1735, 1719, 1638, 1578, 1560, 1524, 1499, 1468, 1422, 1376, 1300, 1248, 1166, 1110, 1053, 1013, 958, 897, 835, 760, 670.

Compound 39: MS (m/z): 1171.37 [MH]⁺,

IR (KBr) cm⁻¹: 3448, 3061, 2971, 2935, 2875, 2787, 1735, 1719, 1702, 1655, 1578, 1560, 1546, 1524, 1492, 1459, 1425, 1376, 1341, 1293, 1167, 1110, 1053, 1012, 959, 897, 764, 697, 669, 625.

### EXAMPLE 37

### Compound 37

### Formula I: M=M5, L=L1, D=D15

To a suspension of compound D15 (70 mg; 0.30 mmol) in dry CH₂Cl₂ (10 mL) under argon, triethylamine (0.380 mL; 2.73 mmol) was added resulting in a clear solution. Subsequently, 1-hydroxybenzotriazole (80 mg; 0.60 mmol), compound M_{L}5 (140 mg; 0.30 mmol) and 1-(3-dimethylaminopropyl)-3-ethyl-carbodiimide hydrochloride (230 mg; 1.20 mmol) were added. The reaction mixture was stirred at room temperature overnight. The solvent was evaporated under reduced pressure and crude product was purified on a silica gel column (eluant: CH₂Cl₂—MeOH—NH₄OH, 90:9:1.5). 44 mg of the compound 37 were obtained.

MS (m/z): 752.19 [MH]⁺,

IR (KBr) cm⁻¹: 3385, 3061, 2972, 2934, 2875, 2127, 1710, 1688, 1637, 1562, 1533, 1470, 1423, 1376, 1353, 1302, 1250, 1163, 1089, 1052, 1000, 958, 897, 760.

### EXAMPLE 38

### Compound 38

### Formula I: M=M7, L=L2, D=D15

To a suspension of compound D15 (30 mg; 0.10 mmol) in dry CH₂Cl₂ (10 mL) under argon, triethylamine (0.140 mL; 1.00 mmol) was added resulting in a clear solution. Subsequently, 1-hydroxybenzotriazole (27 mg; 0.20 mmol), compound M_{L}7 (98 mg; 0.12 mmol) and 1-(3-dimethylaminopropyl)-3-ethyl-carbodiimide hydrochloride (76,4 mg; 0.40 mmol) were added. The reaction mixture was stirred at room temperature overnight. The solvent was evaporated under reduced pressure and crude product was purified on a silica gel column (eluant: CH₂Cl₂—MeOH—NH₄OH, 90:8:1). 12 mg of the compound 38 were obtained.

MS (m/z): 1096.21 [MH]⁺,

IR (KBr) cm⁻¹: 3425, 3057, 2970, 2934, 2870, 1721, 1688, 1639, 1580, 1561, 1525, 1469, 1423, 1377, 1302, 1175, 1110, 1050, 996, 941, 895, 760, 742.

### EXAMPLE 40

### Compound 40

### Formula I: M=M1, L=L1, D=D17

To a suspension of compound D17 (100 mg; approximately 0.16 mmol under 50% purity assumption) in dry CH₂Cl₂ (10 mL) under argon, triethylamine (0.265 mL; 1.9 mmol) was added resulting in a clear solution. Subsequently, 1-hydroxybenzotriazole (51 mg; 0.38 mmol), compound M_{L}1 (150 mg; 0.19 mmol) and 1-(3-dimethylaminopropyl)-3-ethyl-carbodiimide hydrochloride (145.7 mg; 0.76 mmol) were added. The reaction mixture was stirred at room temperature overnight. The solvent was evaporated under reduced pressure and crude product was purified on a silica gel column (eluant: CH₂Cl₂—MeOH—NH₄OH, 90:8:1). 22 mg of the compound 40 were obtained.

MS (m/z): 1093.33 [MH]⁺,

IR (KBr) cm⁻¹: 3432, 3059, 2970, 2935, 2875, 1710, 1656, 1616, 1562, 1544, 1526, 1510, 1469, 1423, 1377, 1326, 1246, 1166, 1107, 1076, 1053, 1012, 960, 836, 761.

### EXAMPLE 41

### Compound 41

### Formula I: M=M3, L=L1, D=D14

To a suspension of compound D14 (28 mg; 0.09 mmol) in dry CH₂Cl₂ (10 mL) under argon, triethylamine (0.118 mL; 0.85 mmol) was added resulting in a clear solution. Subsequently, 1-hydroxybenzotriazole (23 mg; 0.17 mmol), compound M_{L}3 (70 mg; 0.09 mmol) and 1-(3-dimethylaminopropyl)-3-ethyl-carbodiimide hydrochloride (65.5 mg; 0.34 mmol) were added. The reaction mixture was stirred at room temperature overnight. The solvent was evaporated under reduced pressure and crude product was purified on a silica gel column (eluant: CH₂Cl₂—MeOH—NH₄OH, 90:8:1). 25 mg of the compound 41 were obtained.

MS (m/z): 1130.23 [MH]⁺.

### EXAMPLE 42

### Compound 42

### Formula I: M=M1, L=L1, D=D18

To a suspension of compound D18 (50 mg; 0.15 mmol) in dry CH₂Cl₂ (3 mL) under argon, triethylamine (0.183 mL; 1.31 mmol) was added resulting in a clear solution. Subsequently, 1-hydroxybenzotriazole (39 mg; 0.29 mmol), compound M_{L}1 (116 mg; 0.15 mmol) and 1-(3-dimethylaminopropyl)-3-ethyl-carbodiimide hydrochloride (112 mg; 0.58 mmol) were added. The reaction mixture was stirred for 16 hours at room temperature. The solvent was evaporated under reduced pressure and the residue purified on a silica gel column (eluant: CH₂Cl₂—MeOH—NH₄OH, 90:9:1.5). 84 mg of the compound 42 were obtained.

MS (m/z): 1115.29 [MH]⁺,

IR (KBr) cm⁻¹: 3448, 3062, 2972, 2937, 2881, 2834, 2788, 1719, 1655, 1578, 1561, 1543, 1494, 1458, 1447, 1405, 1377, 1250, 1221, 1167, 1094, 1054, 1013, 958, 900, 836, 815, 770, 713, 669.

### EXAMPLE 43

### Compound 43

### Formula I: M=M7, L=L2, D=D18

To a suspension of compound D18 (50 mg; 0.15 mmol) in dry CH₂Cl₂ (3 mL) under argon, triethylamine (0.183 mL; 1.31 mmol) was added resulting in a clear solution. Subsequently, 1-hydroxybenzotriazole (39 mg; 0.29 mmol), compound M_{L}7 (120 mg; 0.15 mmol) and 1-(3-dimethylaminopropyl)-3-ethyl-carbodiimide hydrochloride (112 mg; 0.58 mmol) were added. The reaction mixture was stirred for 16 hours at room temperature. The solvent was evaporated under reduced pressure and the residue purified twice on a silica gel columns (eluants: CH₂Cl₂—MeOH—NH₄OH, 90:9:1.5; then CH₂Cl₂—MeOH—NH₄OH, 90:8:1). 56 mg of the compound 43 were obtained.

MS (m/z): 1115.29 [MH]⁺,

IR (KBr) cm⁻¹: 3447, 2971, 2936, 2875, 2831, 1729, 1655, 1578, 1561, 1542, 1494, 1447, 1405, 1379, 1271, 1221, 1176, 1109, 1095, 1053, 995, 959, 878, 836, 813, 771, 739, 713, 670, 640.

### EXAMPLE 44

### Compound 44

### Formula I: M=M11, L=L1, D=D19

Compound M_{L}5 (89.7 mg; 0.19 mmol) was dissolved in CH₃CN (5 mL) and then compound D19 (68 mg; 0.19 mmol) and K₂CO₃ (39 mg; 0.28 mmol) were added. The reaction mixture was stirred at 80° C. overnight. The solvent was evaporated under reduced pressure and crude product was purified on a silica gel column (eluant: CH₂Cl₂—MeOH—NH₄OH, 90:9:1.5). 34 mg of the compound 44 were obtained.

MS (m/z): 740.28 [MH]⁺,

IR (KBr) cm⁻¹: 3424, 2968, 2931, 2874, 1774, 1717, 1655, 1638, 1630, 1604, 1578, 1561, 1493, 1447, 1406, 1375, 1337, 1271, 1243, 1220, 1132, 1100, 1055, 994, 972, 943, 885, 836, 817, 765, 715, 685, 669.

### EXAMPLE 45

### Compound 45

### Formula I: M=M1, L=L1, D=D20

To a suspension of compound D20 (44.4 mg; 0.14 mmol) in dry CH₂Cl₂ (5 mL) under argon, triethylamine (0.150 mL; 1.08 mmol) was added resulting in a clear solution. Subsequently, 1-hydroxybenzotriazole (37.2 mg; 0.28 mmol), compound M_{L}1 (108.7 mg; 0.14 mmol) and 1-(3-dimethylaminopropyl)-3-ethyl-carbodiimide hydrochloride (95 mg; 0.50 mmol) were added. The reaction mixture was stirred at room temperature overnight. The solvent was evaporated under reduced pressure and crude product was purified on a silica gel column (eluant: CH₂Cl₂—MeOH—NH₄OH, 90:9:1.5). 84 mg of the compound 45 were obtained.

MS (m/z): 1097.3 [MH]⁺,

IR (KBr) cm⁻¹: 3438, 2971, 2935, 2875, 2789, 1719, 1655, 1578, 1560, 1544, 1459, 1377, 1255, 1167, 1109, 1093, 1053, 1012, 1001, 959, 902, 836, 760, 641.

### EXAMPLE 46

### Compound 46

### Formula I: M=M11, L=L1, D=D21

Compound M_{L}5 (70 mg; 0.15 mmol) was dissolved in CH₃CN (5 mL) and then compound D21 (50 mg; 0.15 mmol) and K₂CO₃ (30 mg; 0.22 mmol) were added. The reaction mixture was stirred at 80° C. overnight. The solvent was evaporated under reduced pressure and crude product was purified on a silica gel column (eluant: CH₂Cl₂—MeOH—NH₄OH, 90:9:1.5). 32 mg of the compound 46 were obtained.

MS (m/z): 722.25 [MH]⁺,

IR (KBr) cm⁻¹: 3426, 2925, 2854, 1623, 1501, 1484, 1445, 1409, 1365, 1265, 1228, 1146, 1118, 1098, 1039, 1005, 968, 939, 874, 834, 812, 768, 732, 710, 670, 606.

### EXAMPLE 47

### Compound 47

### Formula I: M=M5, L=L1, D=D21

Compound M_{L}5 (70 mg; 0.15 mmol) was dissolved in CH₃CN (5 mL) and then compound D21 (50 mg; 0.15 mmol) was added. The reaction mixture was stirred at 80° C. overnight. The solvent was evaporated under reduced pressure and crude product was purified on a silica gel column (eluant: CH₂Cl₂—MeOH—NH₄OH, 90:9:1.5). 97 mg of the compound 47 were obtained.

MS (m/z): 740.26 [MH]⁺,

IR (KBr) cm⁻¹: 3433, 3056, 2974, 2933, 2875, 1719, 1638, 1578, 1560, 1488, 1459, 1427, 1372, 1255, 1231, 1161, 1092, 1045, 1032, 962, 803, 759, 737, 703, 670, 652.

### EXAMPLE 48

### Compound 48

### Formula I: M=M1, L=L1, D=D21

Compound D21 (45 mg, 0.16 mmol) was dissolved in MeOH (10 mL). Compound M_{L}1 (128 mg, 0.16 mmol), NaBH₃CN (10.2 mg) and drop of acetic acid were added. The reaction mixture was stirred at room temperature overnight. The solvent was evaporated under reduced pressure and crude product was purified on a silica gel column (eluant: CH₂Cl₂—MeOH—NH₄OH, 90:9:1.5). 23 mg of the compound 48 were obtained.

MS (m/z): 1053.38 [MH]⁺,

IR (KBr) cm⁻¹: 3388, 3051, 2970, 2930, 2853, 2249, 1719, 1655, 1638, 1585, 1561, 1542, 1498, 1459, 1377, 1278, 1166, 1107, 1082, 1053, 1012, 957, 902, 835, 809, 757, 736, 642.

### EXAMPLE 49

### Compound 49

### Formula I: M=M5, L=L1, D=D22

Compound D22 (35 mg, 0.13 mmol) was dissolved in MeOH (10 mL). Compound M_{L}5 (60 mg, 0.13 mmol), NaBH₃CN (8.2 mg) and drop of acetic acid were added. The reaction mixture was stirred at room temperature overnight. The solvent was evaporated under reduced pressure and crude product was purified on a silica gel column (eluant: CH₂Cl₂—MeOH—NH₄OH, 90:9:1.5). 8 mg of the compound 49 were obtained.

MS (m/z): 738.22 [MH]⁺,

IR (KBr) cm⁻¹: 3423, 2954, 2925, 2852, 1774, 1710, 1686, 1655, 1638, 1629, 1578, 1561, 1546, 1499, 1459, 1421, 1376, 1256, 1169, 1081, 1054, 1035, 958, 896, 807, 758, 670.

### EXAMPLE 50

### Compound 50

### Formula I: M=M5, L=L1, D=D23

To the solution of compound D23 (50 mg; 0.16 mmol) and compound M_{L}5 (77 mg; 0.16 mmol) in absolute EtOH (30 mL) was added palladium, 10% on carbon (50 mg) as a catalyst. The mixture was hydrogenated for 20 hours at 5 bar. 150 mg of the raw product was obtained following filtration and evaporation, it was purified on a silica gel column (eluent: CH₂Cl₂—MeOH—NH₄OH, 90:8:1). 40 mg of the compound 50 were obtained.

MS (m/z): 769.2 [MH]⁺.

### EXAMPLE 51

### Compound 51

### Formula I: M=M5, L=L1, D=D24

To a suspension of compound D24 (100 mg; 0.28 mmol) in dry CH₂Cl₂ (5 mL) under argon, triethylamine (0.390 mL; 2. mmol) was added resulting in a clear solution. Subsequently, 1-hydroxybenzotriazole (77 mg; 0.57 mmol), compound M_{L}5 (136 mg; 0.28 mmol) and 1-(3-dimethylaminopropyl)-3-ethyl-carbodiimide hydrochloride (219 mg; 1.14 mmol) were added. The reaction mixture was stirred at room temperature for 4 hours. The solvent was evaporated under reduced pressure and crude product was purified on a silica gel column (eluant: CH₂Cl₂—MeOH—NH₄OH, 90:8:1). 180 mg of the compound 51 were obtained.

MS (m/z): 809.1 [MH]⁺.

1. A compound of the Formula I: 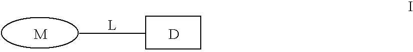
wherein
M represents a macrolide subunit;
D represents a dibenzo[e,h]azulene subunit;
L is a linker molecule to which each of M and D are covalently linked; or
pharmaceutically acceptable salts or solvates thereof or individual diastereoisomers thereof.

2. A compound as claimed in claim 1 wherein M represents a group of the Formula II: 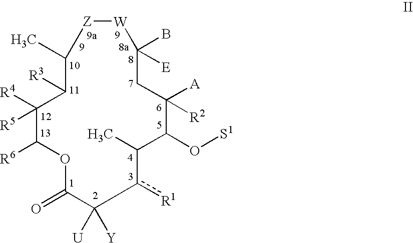
wherein:
(i) Z and W independently are: >C═O, >CH₂, >CH—NRₜRₛ, >N—R_{N} or >C═N—R_{M} or a bond wherein:
Rₜ and Rₛ independently are hydrogen or alkyl;
R_{M} is hydroxy, alkoxy, substituted alkoxy or OR^{p};
R_{N} is hydrogen, R^{p}, alkyl, alkenyl, alkynyl, alkoxy, alkoxyalkyl, or —C(X)—NRₜRₛ; wherein X is ═O or ═S;
provided that Z and W cannot both simultaneously be, >C═O, >CH₂, >CH—NRₜRₛ, >N—R_{N} or >C═N—R_{M} or a bond,
(ii) U and Y independently are hydrogen, halogen, alkyl, or hydroxyalkyl;
(iii) R¹ is hydroxy, OR^{p}, —O—S² group or an ═O;
(iv) S¹ is H or a sugar moiety of formula: 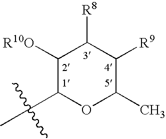
wherein
R⁸ and R⁹ are both hydrogen or together form a bond, or R⁹ is hydrogen and R⁸ is —N(CH₃)R^{y}, wherein
R^{y} is R^{p}, R^{z} or —C(O)R^{z} wherein R^{z} is hydrogen, alkyl, alkenyl, alkynyl, cycloalkyl, aryl, heteroaryl, or alkyl substituted with C₂-C₇-alkyl, C₂-C₇-alkenyl,
C₂-C₇-alkynyl, aryl or heteroaryl
R¹⁰ is hydrogen or R^{p};
(v) S² is H or a sugar moiety of the formula: 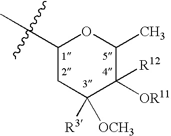
wherein:
R^{3′} is hydrogen or methyl;
R¹¹ is hydrogen, R^{p} or O—R¹¹ is a group that with R¹² and with C/4″ carbon atom forms a >C═O or epoxy group;
R¹² is hydrogen or a group that with O—R¹¹ group and with C/4″ carbon atom forms a >C═O or epoxy group;
(vi) R² is hydrogen, hydroxy, OR^{p} or alkoxy,
(vii) A is hydrogen or methyl;
(viii) B is methyl or epoxy;
(ix) E is hydrogen or halogen;
(x) R³ is hydroxy, OR^{p}, alkoxy or R³ is a group that with R⁵ and with C/11 and C/12 carbon atoms forms a cyclic carbonate or carbamate; or if W or Z is >N—R_{N}, R³ is a group that with W or Z forms a cyclic carbamate;
(xi) R⁴ is C₁-C₄ alkyl;
(xii) R⁵ is hydrogen, hydroxy, OR^{p}, C₁-C₄-alkoxy, or a group that with R³ and with C/11 and C/12 carbon atoms forms a cyclic carbonate or carbamate;
(xiii) R⁶ is hydrogen or C₁-C₄-alkyl; and
R^{p} is a hydroxyl or amino protective group;
wherein M has a linkage site through which it is linked to D via linking group L.

3. A compound according to claim 2 wherein the linkage site is at one or more of the following:
a) any reactive hydroxy, nitrogen, or epoxy group located on S¹, S², or an aglycone oxygen if S¹ or/and S² is cleaved off,
b) a reactive >N—R_{N} or —NRₜRₛ or ═O group located on Z or W;
c) a reactive hydroxy group located at any one of R¹, R², R³, and R⁵; and
d) any other group that can be first derivatized to a hydroxy or —NRₜRₛ, group.

4. A compound as claimed in claim 1 wherein L represents a group of Formula IV:
X¹—(CH₂)ₘ-Q-(CH₂)ₙ—X² IV
wherein
X¹ is selected from: —CH₂—, —C(O)—, OC(O)—, N—O—, —OC(O)NH— or —C(O)NH—;
X² is —NH— or —NHC(O)—, —OC(O)—, —C(O)—, —O or —CH₂—;
Q is —NH— or —CH₂—, or absent;
wherein each —CH₂— or —NH— group may be optionally substituted by C₁-C₇-alkyl,
C₂-C₇-alkenyl, C₂-C₇-alkynyl, C(O)R^{x}, C(O)OR^{x}, or C(O)NHR^{x},
wherein R^{x} is C₁-C₇-alkyl, aryl or heteroaryl; and
m and n independently are a whole number from 0 to 4, with the proviso that if Q is NH, n cannot be 0.

5. A compound as claimed in claim 1 wherein D is derived from the dibenzo[e,h]azulene subunits represented by the Formula III: 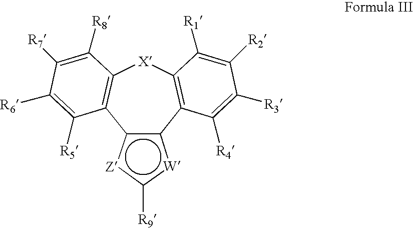
wherein,
X¹ individually denotes a hetero atom —O—; —S—; —CH₂— or NR_{10′};
W′ and Z′ are independently —CH—, S, O or NR_{11′}, with proviso that W and Z can not simultaneously be —CH—, oxygen, or sulfur;
R₁′, R₂′, R₃′, R₄′, R₅′, R₆′, R₇′ and R₈′ independently from each other denote hydrogen or one or more identical or different substituents linked to any available carbon atom, and may be halogen, C₁-C₄ alkyl, halo-C₁-C₄ alkyl, hydroxy, C₁-C₄ alkyoxy, C₁-C₄ alkanoyl, methansulfoanilid, amino, amino-C₁-C₄ alkyl, N—(C₁-C₄-alkyl)amino, N,N-di(C₁-C₄alkyl)amino, thiol, C₁-C₄ alkylthio, hydroxycarbonyl, formyl, cyano, C₁-C₄ alkyloxycarbonyl, C₁-C₇ alkylsulfonyl, C₁-C₇ alkylsulfinyl; hydroxy-C₁-C₇ alkylsulfonyl, hydroxy-C₁-C₇ alkylsulfinyl; amino-C₁-C₇ alkylsulfonyl, amino-C₁-C₇ alkylsulfinyl;
R₉′ is hydrogen, halo, an optionally substituted C₁-C₇ alkyl or C₂-C₇ alkenyl, C₂-C₇ alkynyl group, an optionally substituted aryl, heteroaryl or heterocyclic group, hydroxy, hydroxyalkyl, formyl, hydroxy-C₂-C₇ alkenyl, hydroxy-C₂-C₇ alkynyl, C₁-C₇ alkoxy, C₁-C₇ alkyloxoalkyl, thiol, thio-C₂-C₇ alkenyl, thio-C₂-C₇ alkynyl, C₁-C₇ alkylthiol, methansulfoanilide, amino, N—(C₁-C₇-alkyl)amino, N,N-di(C₁-C₇-alkyl)amino, C₁-C₇ alkylamino, amino-C₂-C₇ alkenyl, amino-C₂-C₇ alkynyl, amino-C₁-C₇ alkoxy, C₁-C₇ alkanoyl, aroyl, oxo-C₁-C₇ alkyl, C₁-C₇ alkanoyloxy, carboxy, an optionally substituted C₁-C₇ alkyloxycarbonyl or aryloxycarbonyl, carbamoyl, N—(C₁-C₇-alkyl)carbamoyl, N,N-di(C₁-C₇-alkyl)carbamoyl, hydroxycarbonylalkyl, cyano, cyano-C₁-C₇ alkyl, sulfonyl, C₁-C₇ alkylsulfonyl, sulfinyl, C₁-C₇ alkylsulfinyl, hydroxy-C₁-C₇ alkylsulfonyl, hydroxy-C₁-C₇ alkylsulfinyl; amino-C₁-C₇ alkylsulfonyl, amino-C₁-C₇ alkylsulfinyl and nitro group or a substituent represented with the formula IIb:
Q₁-(CH₂)ₙ—Q₂-A′ IIb
wherein
Q₁ and Q₂ are independently oxygen, sulfur, or a group: 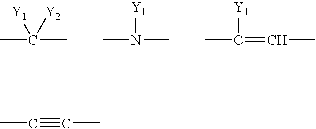
wherein the substituent
y₁ and y₂ independently from each other have the meaning of hydrogen, halogen, an optionally substituted C₁-C₄-alkyl or aryl wherein an optionally substituted alkyl or aryl have the meaning as defined above, hydroxy, C₁-C₄-alkoxy, C₁-C₄-alkanoyl, thiol, C₁-C₄-alkylthio, sulfonyl, C₁-C₄-alkylsulfonyl, sulfinyl, C₁-C₄-alkylsulfinyl, cyano, nitro, or together form a carbonyl or imino group; and
A′ is an amino, N—(C₁-C₇-alkyl)amino, N,N-di(C₁-C₇-alkyl)amino, optionally substituted aryl, heterocyclic or heteroaryl selected from the group consisting of morpholine-4-yl, piperidine-1-yl, pyrrolidine-1-yl, imidazole-1-yl and piperazine-1-yl; or
A′ is represented by structure IIIb; 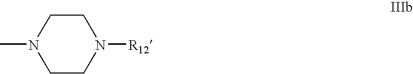
where R₁₂′ denotes hydrogen or an optionally substituted C₁-C₇ alkyl or C₂-C₇ alkenyl, C₂-C₇ alkynyl group, an optionally substituted aryl, heteroaryl or heterocyclic group, C₁-C₇ alkoxy, C₁-C₇ alkylthiol, C₁-C₇ alkanoyl, aroyl, oxo-C₁-C₇ alkyl, C₁-C₇ alkanoyloxy, carboxy, an optionally substituted C₁-C₇ alkyloxycarbonyl or aryloxycarbonyl, carbamoyl, N—(C₁-C₇-alkyl)carbamoyl, N,N-di(C₁-C₇-alkyl)carbamoyl, cyano-C₁-C₇ alkyl, C₁-C₇ alkylsulfonyl, C₁-C₇ alkylsulfinyl;
n denotes an integer from 0 to 5;
R₁₀ denotes hydrogen or an optionally substituted C₁-C₇ alkyl or C₂-C₇ alkenyl, C₂-C₇ alkynyl group, an optionally substituted aryl, heteroaryl or heterocyclic group, C₁-C₇ alkoxy, C₁-C₇ alkylthiol, C₁-C₇ alkanoyl, aroyl, oxo-C₁-C₇ alkyl, C₁-C₇ alkanoyloxy, carboxy, an optionally substituted C₁-C₇ alkyloxycarbonyl or aryloxycarbonyl, arylalkyl, carbamoyl, N—(C₁-C₇-alkyl)carbamoyl, N,N-di(C₁-C₇-alkyl)carbamoyl, cyano-C₁-C₇ alkyl, C₁-C₇ alkylsulfonyl, C₁-C₇ alkylsulfinyl;
R₁₁′ denotes hydrogen or an optionally substituted C₁-C₇ alkyl or C₂-C₇ alkenyl, C₂-C₇ alkynyl group, an optionally substituted aryl, heteroaryl or heterocyclic group, C₁-C₇ alkoxy, C₁-C₇ alkylthiol, C₁-C₇ alkanoyl, aroyl, oxo-C₁-C₇ alkyl, C₁-C₇ alkanoyloxy, arylalkyl, carboxy, an optionally substituted C₁-C₇ alkyloxycarbonyl or aryloxycarbonyl, carbamoyl, N—(C₁-C₇-alkyl)carbamoyl, N,N-di(C₁-C₇-alkyl)carbamoyl, cyano-C₁-C₇ alkyl, C₁-C₇ alkylsulfonyl, C₁-C₇ alkylsulfinyl;
as well as pharmacologically acceptable esters, salts or solvates thereof.

6. A compound as claimed in claim 2 wherein Z and W in the group of Formula II together are:
—N(CH₃)—CH₂—, —NH—CH₂—, —CH₂—NH—, —C(O)—NH— or —NH—C(O)—;
A and B are methyl;
E is hydrogen;
R² is hydroxy or methoxy;
S¹ represents desosamine sugar wherein R⁸ is selected from: hydrogen, methyl,
amino, C₁-C₆ alkylamino or C₁-C₆ dialkylamino;
R⁹ and R¹⁰ are hydrogen;
R¹ is hydroxy or the O—S² group wherein the S² represents a cladinose sugar wherein:
R¹¹ is hydrogen, or O—R¹¹ is a group that with R¹² and with C/4″ carbon atom forms a >C═O or epoxy group; R¹² is hydrogen or a group that with O—R¹¹ and with C/4″ carbon atom forms a >C═O or epoxy group;
R¹³ is methyl;
U is hydrogen
Y is methyl;
R⁶ is hydroxy, methyl or ethyl;
R⁵ is hydrogen, hydroxy, methoxy or a group that with R³ and with C/11 and C/12 carbon atoms forms a cyclic carbonate or carbamate bridge;
R³ is hydroxy or a group that forms a cyclic carbamate bridge with W or Z, or R³ is a group that with R⁵ and with C/11 and C/12 carbon atoms forms a cyclic carbonate or carbamate bridge;
R⁴ is methyl; and
provided that the linkage is through the nitrogen of Z at N/9a position or through the carbon of R¹² or through the oxygen of R¹¹ both at C/4″ position of the S² sugar.

7. A compound as claimed in claim 4 wherein
X¹ is —CH₂— or —OC(O)—;
X² is —NHC(O)—; and
Q is —NH— or absent.

8. The compound of claim 1, having the formula:

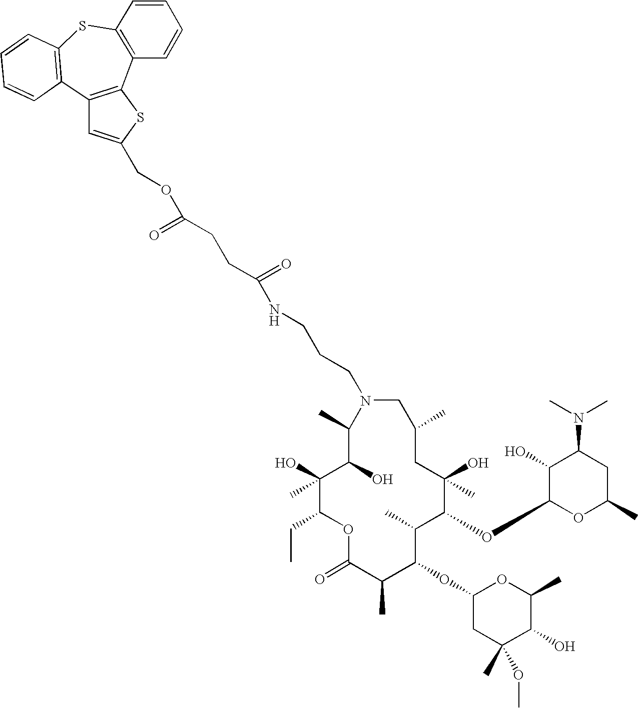

9. The compound of claim 1, having the formula:

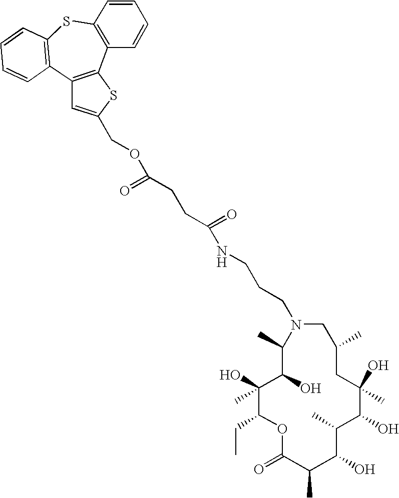

10. The compound of claim 1, having the formula:

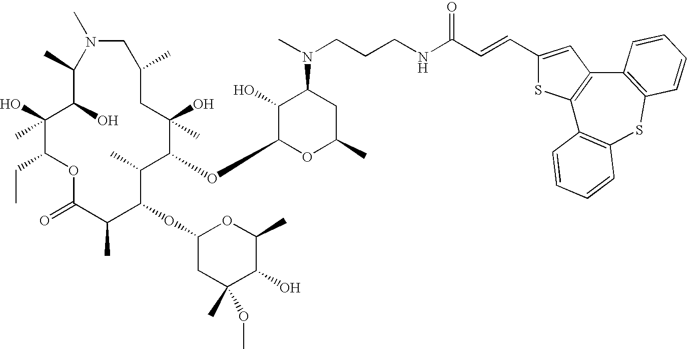

11. The compound of claim 1, having the formula:

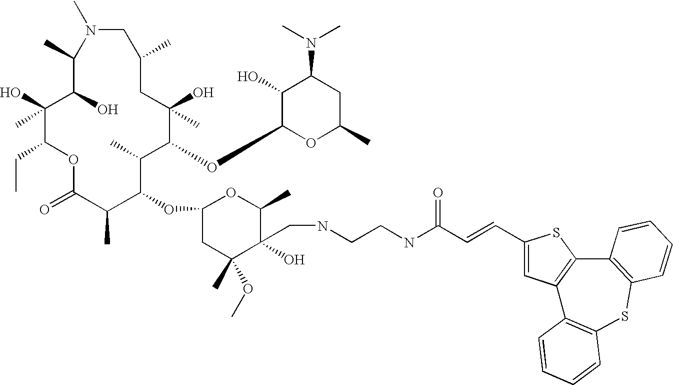

12. The compound of claim 1, having the formula:

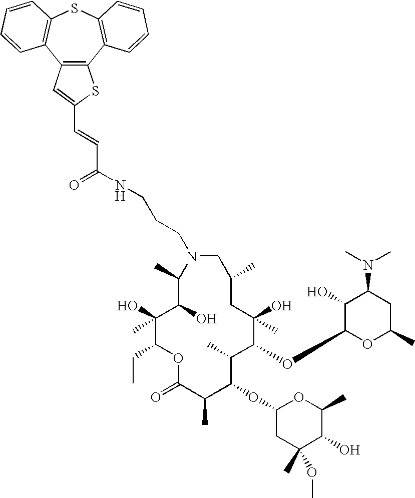

13. The compound of claim 1, having the formula:

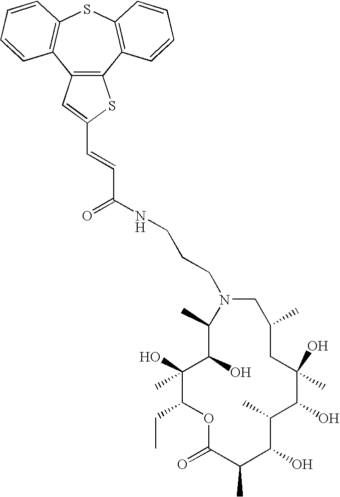

14. The compound of claim 1, having the formula:

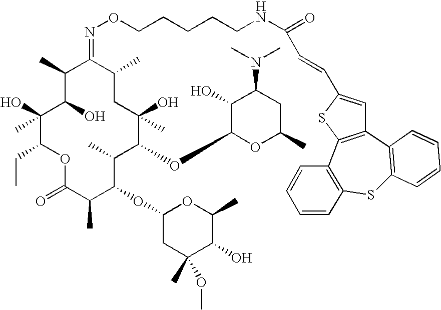

15. The compound of claim 1, having the formula:

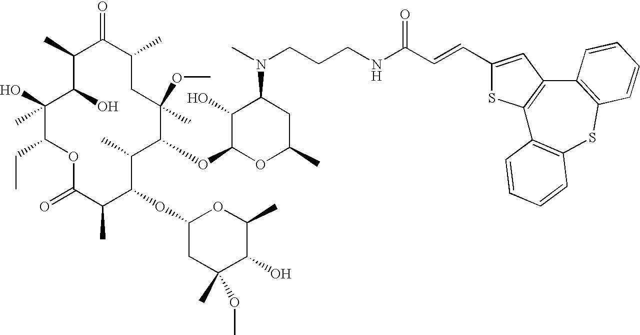

16. The compound of claim 1, having the formula:

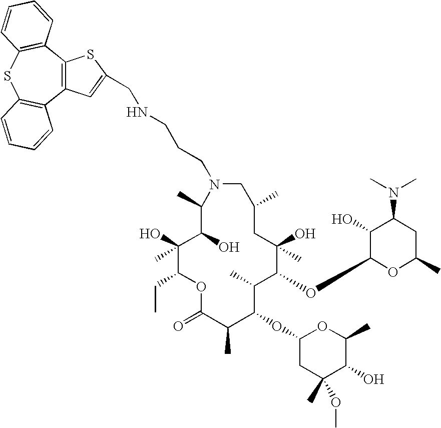

17. The compound of claim 1, having the formula:

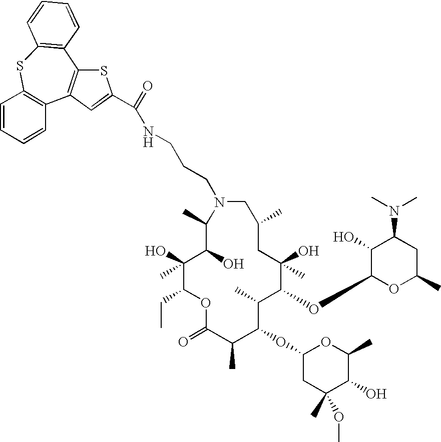

18. The compound of claim 1, having the formula:

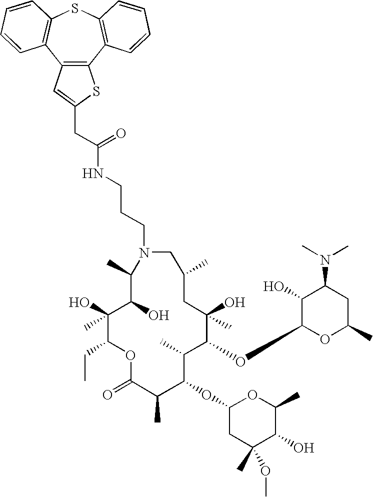

19. The compound of claim 1, having the formula:

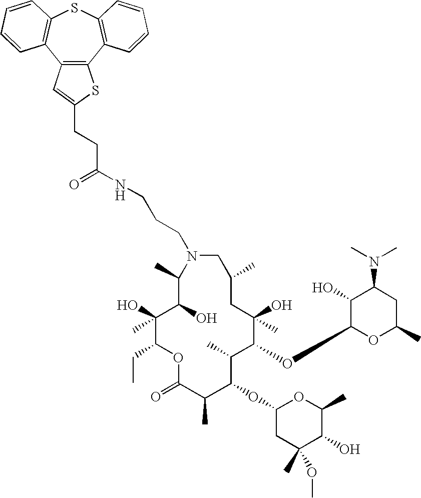

20. The compound of claim 1, having the formula:

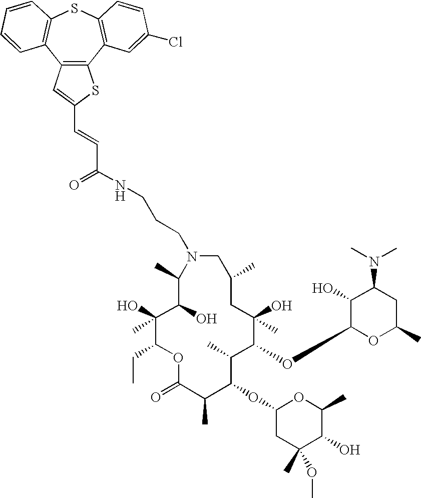

21. The compound of claim 1, having the formula:

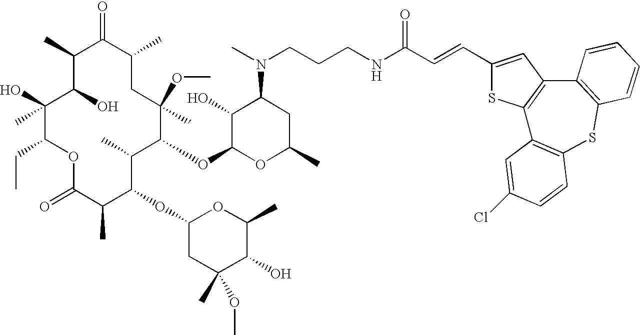

22. The compound of claim 1, having the formula:

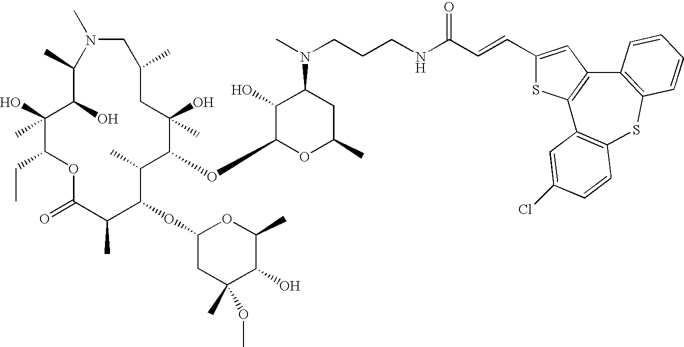

23. The compound of claim 1, having the formula:

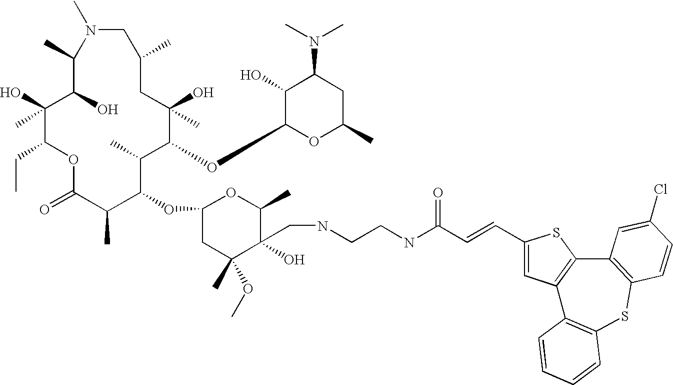

24. The compound of claim 1, having the formula:

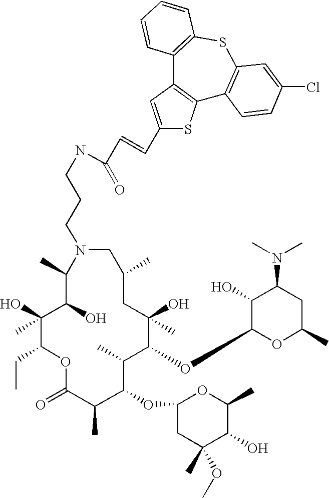

25. The compound of claim 1, having the formula:

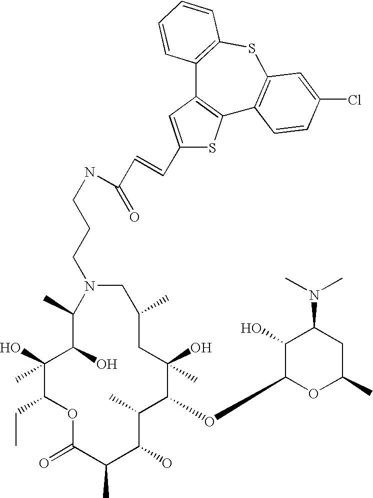

26. The compound of claim 1, having the formula:

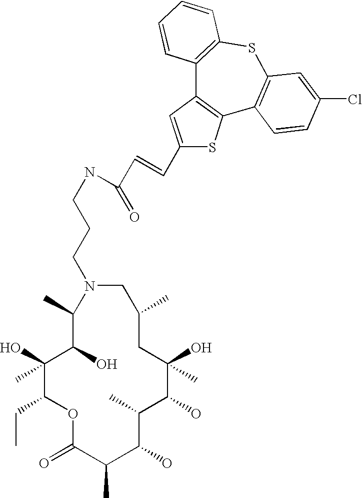

27. The compound of claim 1, having the formula:

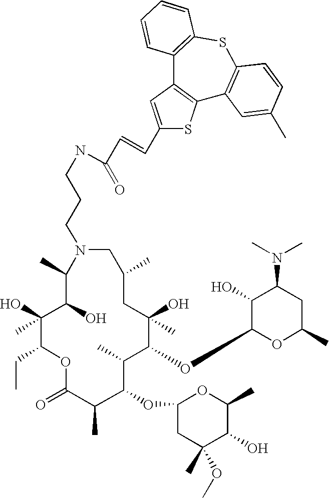

28. The compound of claim 1, having the formula:

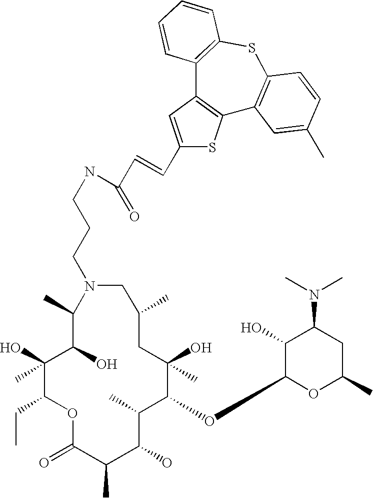

29. The compound of claim 1, having the formula:

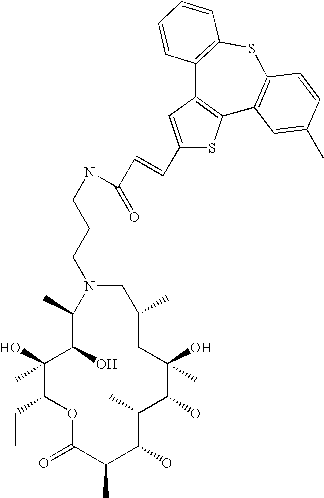

30. The compound of claim 1, having the formula:

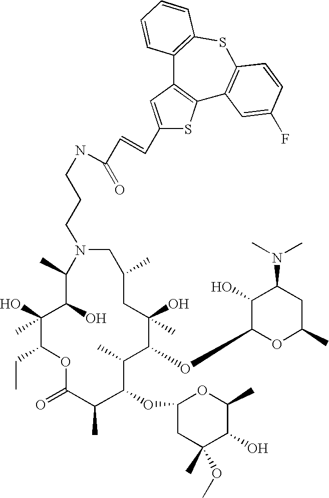

31. The compound of claim 1, having the formula:

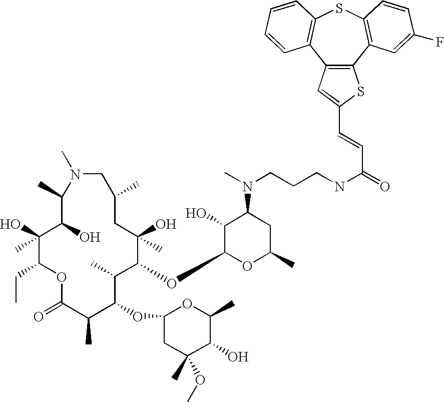

32. The compound of claim 1, having the formula:

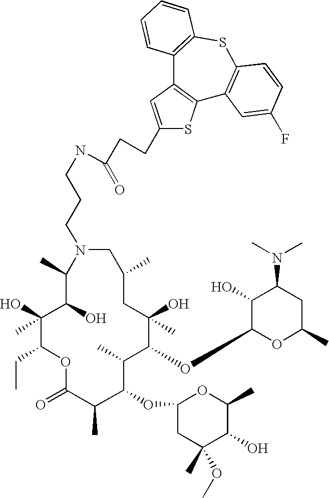

33. The compound of claim 1, having the formula:

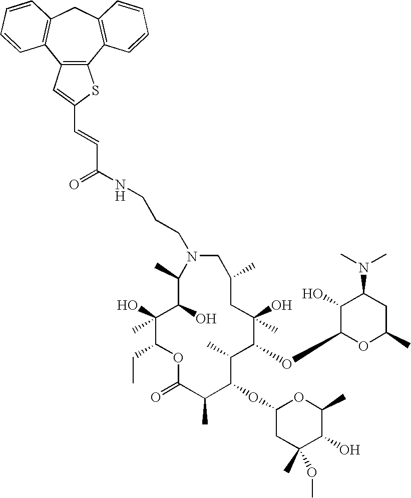

34. The compound of claim 1, having the formula:

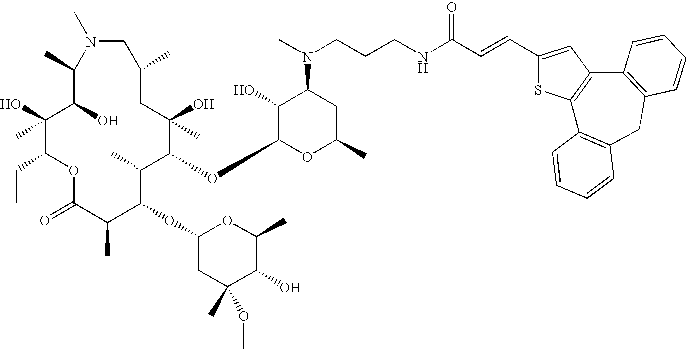

35. The compound of claim 1, having the formula:

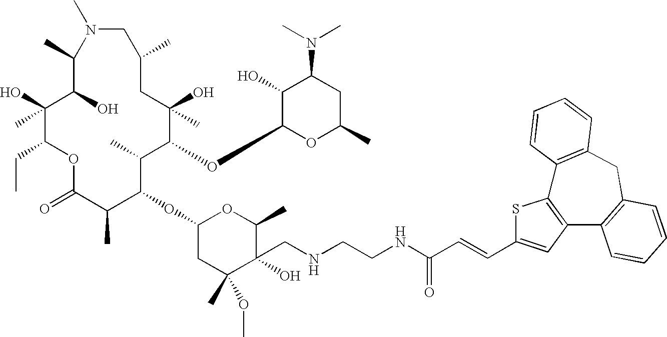

36. The compound of claim 1, having the formula:

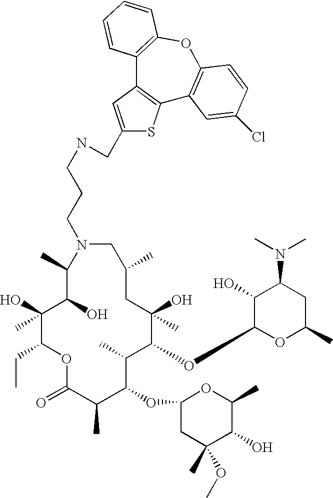

37. The compound of claim 1, having the formula:

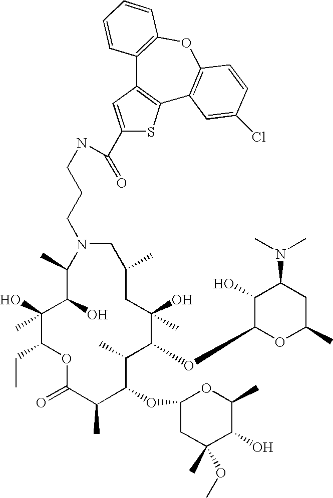

38. The compound of claim 1, having the formula:

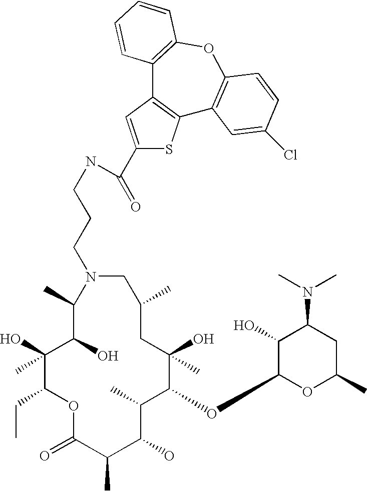

39. The compound of claim 1, having the formula:

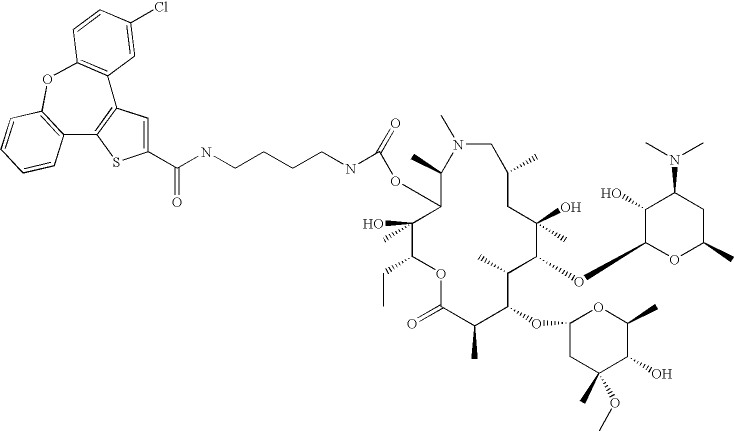

40. The compound of claim 1, having the formula:

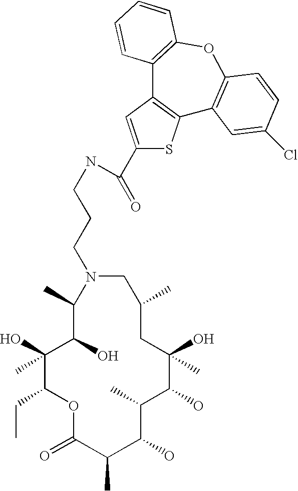

41. The compound of claim 1, having the formula:

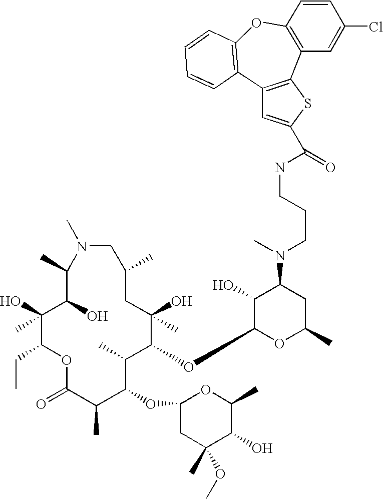

42. The compound of claim 1, having the formula:

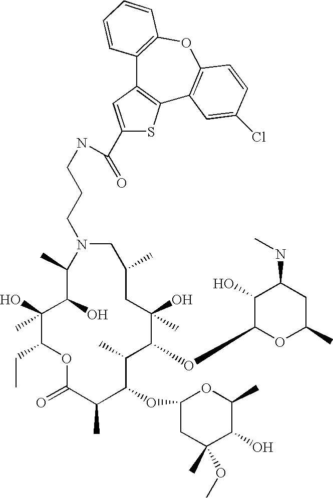

43. The compound of claim 1, having the formula:

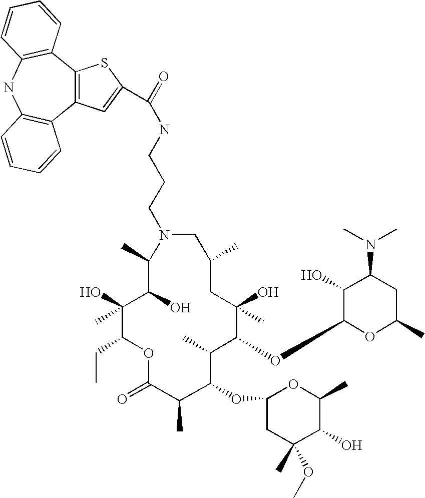

44. The compound of claim 1, having the formula:

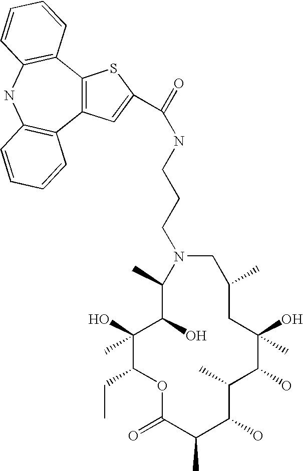

45. The compound of claim 1, having the formula:

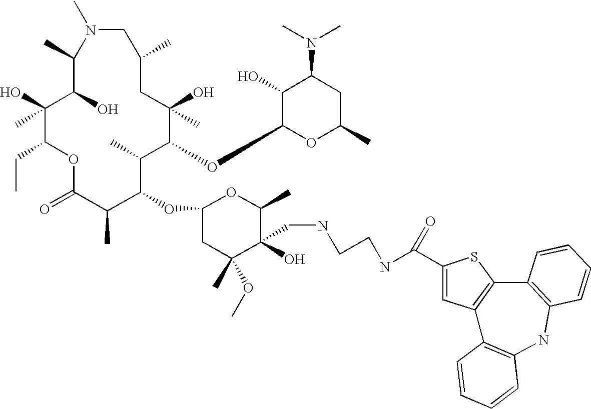

46. The compound of claim 1, having the formula:

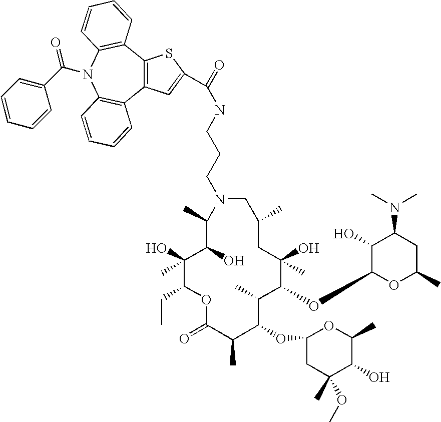

47. The compound of claim 1, having the formula:

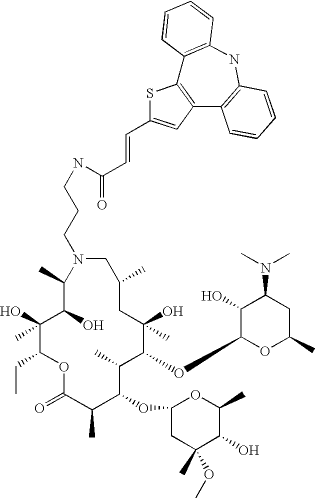

48. The compound of claim 1, having the formula:

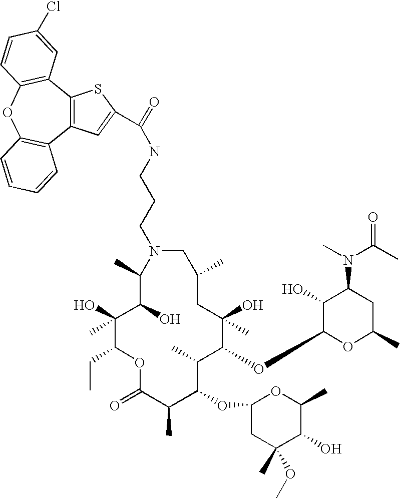

49. The compound of claim 1, having the formula:

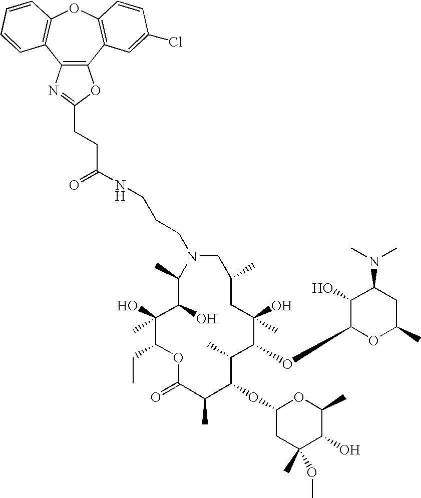

50. The compound of claim 1, having the formula:

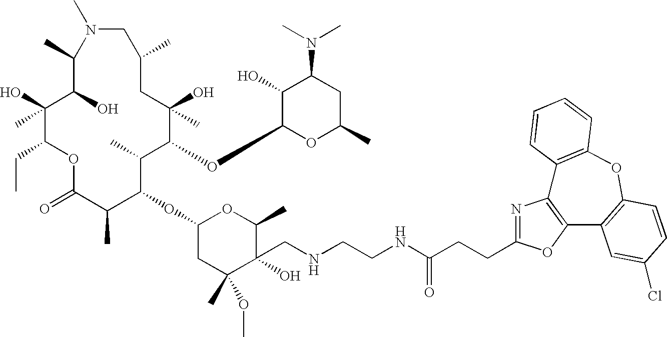

51. The compound of claim 1, having the formula:

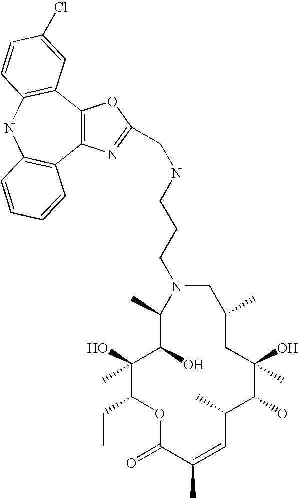

52. The compound of claim 1, having the formula:

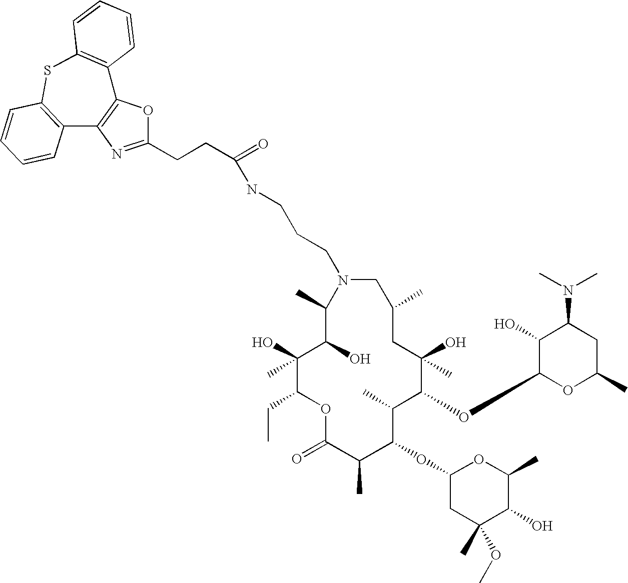

53. The compound of claim 1, having the formula:

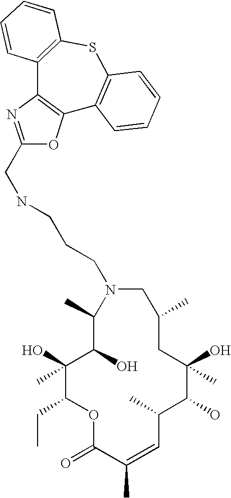

54. The compound of claim 1, having the formula:

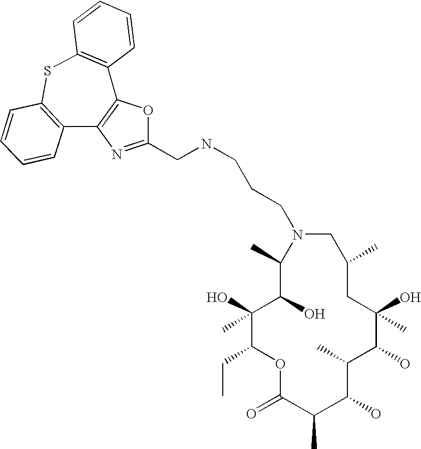

55. The compound of claim 1, having the formula:

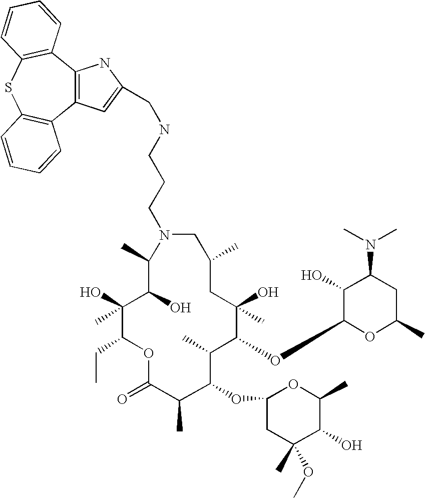

56. The compound of claim 1, having the formula:

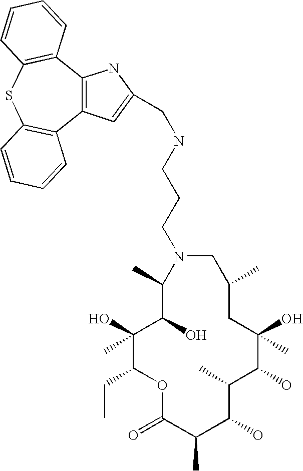

57. The compound of claim 1, having the formula:

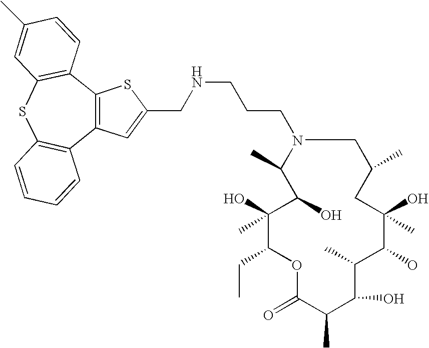

58. The compound of claim 1, having the formula:

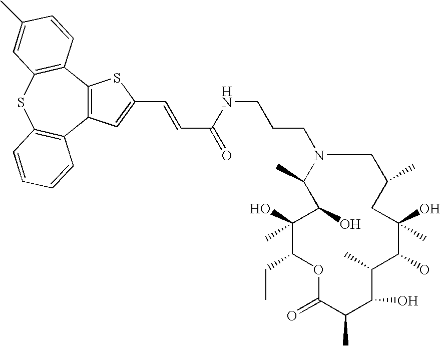

59. The compound of claim 5, wherein L and R⁹′ together comprise:
—(CH₂)ₙ—NH—C(O)—CH═CH—;
—(CH₂)ₙ—NH—C(O)—(CH₂)ₙ—;
—(CH₂)ₙ—NH—C(O)—(CH₂)ₙ—C(O)—O—(CH₂)ₙ—; or —(CH₂)ₙ—NH—(CH₂)ₙ—;
wherein n is 2-3.

60. The compound of claim 5, wherein R₁′, R₂′, R₃′, R₄′, R₅′, R₆′, R₇′ and R₈′ independently from each other denote hydrogen or one or more identical or different substituents linked to any available carbon atom, and may be halogen, or C₁-C₄ alkyl.

61. The compound of claim 5, wherein one of W′ and Z′ is S and the other of W′ and Z′ is —CH—.

62. The compound of claim 5, wherein:
X′ individually denotes a hetero atom —O—; —S—; —CH₂—, NH, or NC(O)-aryl;
W′ and Z′ are independently —CH—, S, O, or NH, with proviso that W and Z can not simultaneously be —CH—, S or O; and
R₁′, R₂′, R₃′, R₁, R₅′, R₆′, R₇′ and R₈′ independently from each other denote hydrogen or one or more identical or different substituents linked to any available carbon atom, and may be halogen or C₁-C₄ alkyl.

63. A process for the preparation a compound of Formula I: 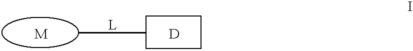
wherein L represents a group of Formula IV:
X¹—(CH₂)ₘ-Q-(CH₂)ₙ—X² IV
wherein
X¹ is selected from: —CH₂—, —C(O)—, OC(O)—, N—O—, —OC(O)NH— or —C(O)NH—;
X² is —NH— or —NHC(O)—, —OC(O)—, —C(O)—, —O or —CH₂—; and
Q is —NH— or —CH₂—, or absent;
wherein each —CH₂— or —NH— group may be optionally substituted by C₁-C₇-alkyl, C₂-C₇-alkenyl, C₂-C₇-alkynyl, C(O)R^{x}, C(O)OR^{x}, or C(O)NHR^{x},
wherein R^{x} is C₁-C₇-alkyl, aryl or heteroaryl; and
m and n independently are a whole number from 0 to 4, with the proviso that if Q is NH, n cannot be 0;
which comprises one of steps (a)-(f):
a) for a compound of Formula I, where X² is —NHC(O)—, by reacting a compound of Formula V: 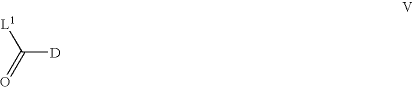
wherein L¹ represents a leaving group, and a free amino group of a macrolide represented by Formula VIa: 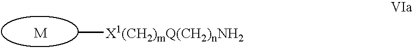
b) for a compound of Formula I, where X² is —OC(O)—, by reacting a compound of Formula V and the free hydroxyl group of a macrolide represented by Formula VIb: 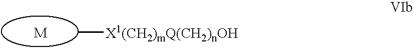
c) for a compound of Formula I, wherein X¹ is —OC(O)—, Q is —NH— and X² is —NHC(O)—, by reacting a macrolide represented by formula: 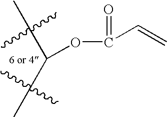
and a free amino group of the compound represented by formula: 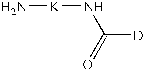
d) for a compound of Formula I, where X¹ is —OC(O)NH— and X² is —NHC(O)—, by reacting a macrolide represented by formula 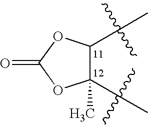
and free amino group of the compound represented by formula: 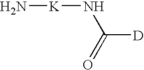
e) for a compound of Formula I, where X¹ is —CH₂—, Q is —NH— and X² is —NHC(O)—, by reacting a macrolide represented by formula: 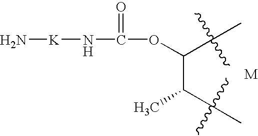
and a compound of Formula V; or
f) for a compound of Formula I by reacting a macrolide represented by
Formula VIIf or by Formula VIIg or by Formula VIIh having a leaving group L² 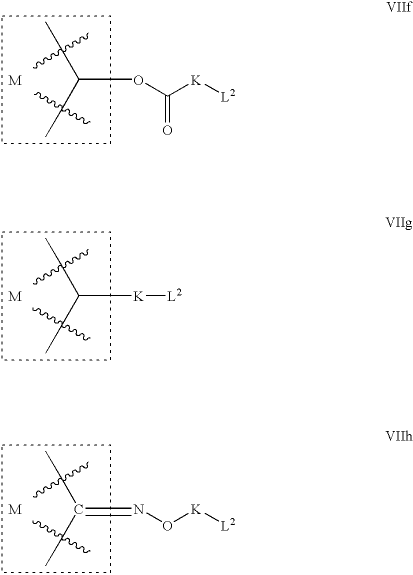
with a free carboxylic acid of dibenzo[e,h]azulene subunit.

64. A pharmaceutical composition comprising a compound as claimed in claim 1 or a pharmaceutically acceptable salt or solvate thereof and a pharmaceutically acceptable diluent or carrier.

65. A method for treatment of an inflammatory disease, disorder, or condition characterized by or associated with an undesirable inflammatory immune response, comprising administering to a subject in need of treatment an effective amount of a compound of claim 1 or a pharmaceutically acceptable salt or solvate thereof.

66. The method of claim 65 wherein said disease, disorder or condition is induced by or associated with excessive secretion of TNF α or IL-1.

67. A method for treatment of an immune or anaphylactic disorder associated with infiltration of leukocytes into inflamed tissue in a subject in need thereof which comprises administering to said subject a therapeutically effective amount of the compound of claim 1 or a pharmaceutically acceptable salt or solvate thereof.

68. The method of claim 67, wherein the disorder is selected from the group consisting of asthma, adult respiratory distress syndrome, bronchitis, and cystic fibrosis.

69. The method of claim 65, wherein said disease, condition, or disorder is selected from the group consisting of inflammatory conditions or immune disorders of the lungs, joints, eyes, bowel, skin, and heart.

70. The method of claim 65, wherein said disorder is selected from the group consisting of asthma, adult respiratory distress syndrome, bronchitis, bronchiectasis, bronchiolitis obliterans, cystic fibrosis, rheumatoid arthritis, rheumatoid spondylitis, osteoarthritis, osteomyelitis, sinusitis, nasal polyps, gouty arthritis, uveitis, conjunctivitis, inflammatory bowel disease, Crohn's disease, ulcerative colitis, distal proctitis, psoriasis, eczema, dermatitis, acne, coronary infarct damage, chronic inflammation, endotoxin shock, chronic sinusitis, pulmonary fibrosis, diffuse panbronchiolitis, and smooth muscle proliferation disorders.

71. A method for reducing or inhibiting inflammation in an affected organ or tissue comprising delivering to said organ or tissue a therapeutically effective amount of the compound of claim 1 or a pharmaceutically acceptable salt as or solvate thereof.

72. A method of inhibiting one or more inflammatory processes selected from the group consisting of: proinflammatory cytokine production, leutkotriene production, 5-lipoxygenase inhibition, prostaglandin production, lung eosinophilia, and immune response associated with shock, and oedema, the method comprising exposing an organ or tissue afflicted with inflammation to an amount of a compound according to claim 1 effective to inhibit said inflammatory process.

73. The method of claim 72, wherein the inflammatory process comprises proinflammatory cytokine production, the method further comprising exposing human peripheral leukocytes to an amount of a compound according to claim 1 effective to reduce production of at least one of TNF-α, IL-1α, IL-1β, IL-6, IL-8, IL-2, IL-5, and IFN-α, compared to control leukocytes.

74. The method of claim 72, wherein the inflammatory process comprises TNF-α and/or IL-1β secretion.

75. The method of claim 72, wherein the inflammatory process comprises leutkotriene production/5-Lox production comprising exposing said organ or tissue to an amount of a compound according to claim 1 effective to reduce production of leukotriene B4 compared to the organ or tissue not exposed to said compound, or prior to being exposed to said compound.

76. The method of claim 72, wherein the inflammatory process causes pain associated with said organ or tissue comprising exposing an organ or tissue to an amount of a compound according to claim 1 effective to induce analgesia.

77. The method of claim 72, wherein the inflammatory process comprises lung eosinophilia.

78. The method of claim 72, wherein the inflammatory process is associated with antigen-induced shock.

79. The method of claim 72, wherein the inhibition of the inflammatory process comprises inhibiting the production of cytokines, leutkotriene B4, prostoglandin E2, eosinophilia, or immune response by at least 50%, wherein the amount of compound according to claim 1 is less than 20 μM.
